(19) United States
(12) Patent Application Publication
(10) Pub. No.: US 2023/0227435 A1
(43) Pub. Date: Jul. 20, 2023

(54) SMALL MOLECULE MODULATORS OF IL-17

(71) Applicant: LEO PHARMA A/S, Ballerup (DK)

(72) Inventors: Mark ANDREWS, Ballerup (DK); Kevin Neil DACK, Ballerup (DK); Jimmi Gerner SEITZBERG, Ballerup (DK); Peter ANDERSEN, Ballerup (DK)

(21) Appl. No.: 18/002,080

(22) PCT Filed: Jun. 17, 2021
(86) PCT No.: PCT/EP2021/066422

§ 371 Date: Dec. 16, 2022

(30) Foreign Application Priority Data
Jun. 18, 2020 (EP) 20180772.4

(51) Int. Cl. C07D 403/12; C07D 401/14; C07D 403/14; C07D 413/14; C07D 405/14

(52) U.S. Cl. CPC C07D 403/12; C07D 401/14; C07D 403/14; C07D 413/14; C07D 405/14

(57) ABSTRACT

The present disclosure relates to compounds according to formula I

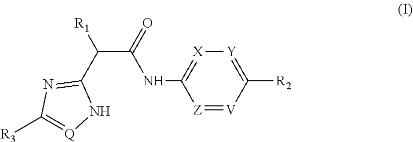

and pharmaceutically acceptable salts, hydrates, or solvates thereof. The disclosure further relates to said compounds for use in therapy, to pharmaceutical compositions comprising said compounds, to methods of treating diseases, e.g. dermal diseases, with said compounds, and to the use of said compounds in the manufacture of medicaments.

### FIELD OF THE INVENTION

This invention relates to novel amino-acid anilides and derivatives thereof, to said compounds for use in therapy and to pharmaceutical compositions comprising said compounds.

### BACKGROUND OF THE INVENTION

IL-17 (also known as IL-17A or CTLA8) is a pro-inflammatory cytokine involved in anti-microbial defense at epithelial surfaces. IL-17 is comprised of two covalently joined IL-17A subunits (IL-17AA) with an approximate mass of 32 kDa, and signals through a receptor comprising IL17RA and IL17RC subunits. This receptor is predominantly expressed in epithelial and mesenchymal cells. The IL17RA/IL17RC receptor is also used by IL-17 variants IL-17AF and IL-17FF, which both are successively weaker, partial agonists on this receptor (Monin, L., Gaffen, S. L.; 2018, Cold Spring Harb. Perspect. Biol. 10. doi:10.1101/cshperspect.a028522). Crucial for signaling is the assembly of signaling complexes containing the multifunctional protein ACT1/CIKS, which in turn can recruit TRAF and other proteins.

Via these signaling complexes IL-17 induces cytokines, chemokines, antimicrobial peptides and growth factors via activation of transcription factor NFkB or via MAP kinase-dependent pathways (e.g. IL-6, IL-8, CXCL1, CXCL2, CXCL5, CCL20, G-CSF, BD4) and stabilizes the mRNAs of certain inflammatory cytokines, such as CXCL1. This leads to amplification of their effects. Further, IL-17 acts in concert with IL-1beta, IL-22 and IFNgamma (Amatya, N. et al., Trends in Immunology, 2017, 38, 310-322. doi:10.1016/j.it.2017.01.006; Onishi, R. M., Gaffen, S. L. Immunology, 2010, 129, 311-321. doi:10.1111/j.1365-2567.2009.03240.x).

IL-17 is secreted by a variety of immune cells, such as Th17 helper cells, Tc17 cytotoxic cells, ILC3 innate cells, NKT cells, TCRbeta+ natural T cells and gamma-deltaT-cells (Monin, L., Gaffen, S. L.; 2018, Cold Spring Harb. Perspect. Biol. 10. doi:10.1101/cshperspect.a028522). Increased, disease-provoking levels of IL-17 are observed in several autoimmune diseases, such as psoriasis, ankylosing spondylitis, spondyloarthritis and psoriatic arthritis. Other diseases where deregulation of IL-17 is observed are rheumatoid arthritis, systemic lupus erythematosus, asthma, inflammatory bowel disease, autoimmune uveitis, multiple sclerosis and certain cancers (Gaffen, S. L. et al., Nat Rev Immunol., 2014, 14, 585-600. doi:10.1038/nri3707; Monin, L., Gaffen, S. L.; 2018, Cold Spring Harb. Perspect. Biol. 10. doi:10.1101/cshperspect.a028522). Hence, IL-17 is a significant therapeutic target.

Therapeutic, neutralizing antibodies against IL-17A (Secukinumab, Ixekizumab) or receptor IL17RA (Brodalumab) have shown high efficacy in the treatment of psoriasis, ankylosing spondylitis and psoriatic arthritis. These antibodies have long half-lives in the body.

Although various antibodies against IL-17A or IL-17RA are approved, there are currently no approved, orally available modulators of IL-17. The following small molecule modulators are known.

WO2013116682 discloses Macrocyclic Compounds for Modulating IL-17;

WO2014066726 discloses Compounds for Modulating IL-17;

WO2018229079 discloses Compounds for Modulating IL-17;

WO2019223718 discloses Compounds for Modulating IL-17;

WO2019138017 discloses Compound for Modulating IL-17;

WO2020011731 discloses Compound for Modulating IL-17;

WO2020120140 discloses Compounds for Modulating IL-17;

WO2020120141 discloses Compounds for Modulating IL-17;

WO2020260426 discloses Compounds for Modulating IL-17;

WO2020260425 discloses Compounds for Modulating IL-17;

WO2020261141 discloses Compounds for Modulating IL-17;

WO2020146194 discloses IL-17A inhibitors.

Chinese patent applications CN112341429A, CN112341435A, CN112341439A, CN112341440A, CN112341441A, CN112341442A, CN112341446A, CN112341450A, CN112341451A and CN112341519A disclose Compounds for Modulating IL-17.

Scientific Reports (2016) 6, 30859 discloses Macrocyclic IL-17A Antagonists. Leslie Dakin, 12^{th} Swiss Course on Medicinal Chemistry, Leysin, Oct. 9-14, 2016 discloses ‘Hit Identification, binding site elucidation and structure guided design of novel macrocyclic IL-17A antagonists’.

Orally available, highly efficacious small molecule IL-17 modulators which bind to IL-17 to decrease its functional ability to activate the IL-17 receptor complex may have a number of advantages compared to monoclonal antibodies. Oral administration and flexible treatment regimen may be two significant aspects in favor of patient convenience and the compounds may exhibit improved safety due to the possibility of faster withdrawal of the drug should adverse events occur.

Therefore, there is a continuous need to develop small molecule modulators of IL-17, particularly small molecules suitable for oral administration.

In addition, some patients may be treated by topical application of small molecule modulators of IL-17. This can be particularly suitable for patients with skin lesions that are readily accessible and limited in body surface area. Topical treatment may also be prescribed for certain patients who could benefit from avoiding systemic modulation of the IL-17 pathway, for example when undergoing treatment for infections or gastrointestinal problems.

### SUMMARY OF THE INVENTION

The inventors have surprisingly found that novel compounds of the present invention exhibit modulating effect on the IL-17 signalling pathway.

Compounds of the present invention may have advantageous properties such as high metabolic stability and/or membrane permeability properties that make them suitable for oral administration. Other compounds of the present invention may have advantageous properties for local topical therapy, such as high skin permeability and high metabolic instability.

Compounds of the present invention may be beneficial in preventing, treating or ameliorating a variety of diseases which involve up-regulation or de-regulation of IL-17, such as for example psoriasis, ankylosing spondylitis and psoriatic arthritis.

Accordingly, the present invention relates to a compound according to formula (I)

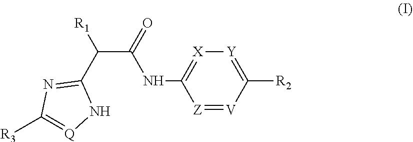

wherein X, Y, Z and V are each independently selected from N, CH and C(R₄);

R₄ is selected from (C₁-C₆)alkyl, (C₁-C₆)alkoxy, amino, hydroxy and halogen, wherein said (C₁-C₆)alkyl and (C₁-C₆)alkoxy is optionally substituted with one of more substituents independently selected from halogen;

Q is C(R₅), or N;

R₁ is selected from the group consisting of —CHR₆R₇, (C₃-C₁₀)cycloalkyl and G, wherein said (C₃-C₁₀)cycloalkyl and G are optionally substituted with one or more substituents independently selected from deuterium, halogen, cyano, (C₁-C₄)alkyl and halo(C₁-C₄)alkyl;

G is

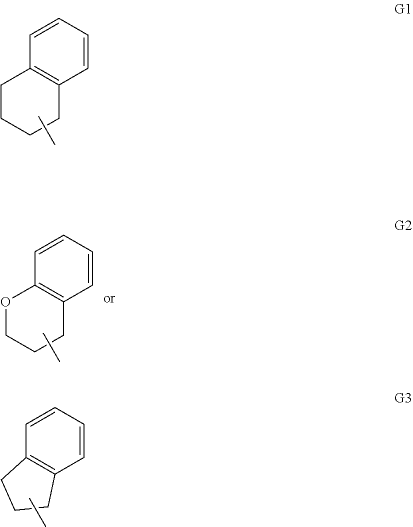

R₆ and R₇ each independently represents hydrogen, phenyl, (C₁-C₆)alkyl, (C₃-C₇)cycloalkyl, or (C₃-C₇)cycloalkyl-(C₁-C₆)alkyl, wherein said phenyl, (C₁-C₆)alkyl, (C₃-C₇)cycloalkyl and (C₃-C₇)cycloalkyl-(C₁-C₆)alkyl, is optionally substituted with one or more substituents independently selected from halogen, cyano, and (C₁-C₄)alkyl; with the proviso that at least one of R₆ and R₇ is different from hydrogen;

R₂ is selected from the group consisting of 5- or 6-membered heteroaryl, wherein said 5-or 6-membered heteroaryl is optionally substituted with one or more substituents independently selected from Rₐ, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

L is selected from the group consisting of a bond or —CHR_{g}O—,

R_{g} is independently selected from hydrogen and (C₁-C₆)alkyl;

R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b};

R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy,

or pharmaceutically acceptable salts, hydrates and solvates thereof.

In one embodiment the present invention relates to compounds of formula (Ia)

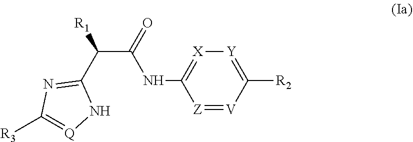

wherein X, Y, Z, V, Q, R₁, R₂ and R₃ are as defined in claim 1 or pharmaceutically acceptable salts, hydrates and solvates thereof.

In another aspect, the invention relates to a pharmaceutical composition comprising a compound of general formula (I) as defined herein together with a pharmaceutically acceptable vehicle or excipient or pharmaceutically acceptable carrier(s), optionally together with one or more other therapeutically active compound(s).

In yet another aspect, the invention relates to the use of a compound according to formula I as defined herein for use in therapy, for example for use in treatment of a disease, disorder or condition, which disease, disorder or condition is responsive of modulation of IL-17, for example for use in treatment of autoimmune diseases.

### DETAILED DESCRIPTION OF THE INVENTION

Definitions

The term “(Cₐ-C_{b})alkyl” is intended to indicate a hydrocarbon radical obtained when one hydrogen atom is removed from a branched or linear hydrocarbon. Said alkyl comprises (a-b) carbon atoms, such as 1-6, such as 1-4, such as 1-3, such as 2-3 or such as 1-2 carbon atoms. The term includes the subclasses normal alkyl (n-alkyl), secondary and tertiary alkyl, such as methyl, ethyl, n-propyl, isopropyl, n-butyl, isobutyl, sec.-butyl, tert.-butyl, n-pentyl, isopentyl, neopentyl, n-hexyl and isohexyl.

The terms “(Cₐ-C_{b})alkoxy” are intended to indicate a radical of the formula —OR′, wherein R′ is (Cₐ-C_{b})alkyl as indicated herein, wherein the (Cₐ-C_{b})alkyl group is appended to the parent molecular moiety through an oxygen atom, e.g. methoxy (—OCH₃), ethoxy (—OCH₂CH₃), n-propoxy, isopropoxy, butoxy, tert-butoxy, and the like.

The term “cyano” is intended to indicate a —CN group attached to the parent molecular moiety through the carbon atom.

The term “(Cₐ-C_{b})cycloalkyl” is intended to indicate a saturated (Cₐ-C_{b})cycloalkane hydrocarbon radical, including polycyclic radicals such as bicyclic or tricyclic radicals, including spirocyclic radicals, comprising a-b carbon atoms, such as 3-10 carbon atoms, such as 3-8 carbon atoms, such as 3-7 carbon atoms, such as 3-6 carbon atoms, such as 3-5 carbon atoms or such as 3-4 carbon atoms, e.g. cyclopropyl, cyclobutyl, cyclopentyl, cyclohexyl, cycloheptyl, cyclooctanyl, adamantyl, spiro[2.5]octanyl, spiro[2,3]hexanyl, bicyclo[3,1,0]hexanyl, bicyclo[4,1,0]heptanyl and bicyclo[2,2,2]octanyl.

The term “(Cₐ-C_{b})cycloalkoxy” is intended to indicate a radical of the formula —OR′, wherein R′ is (Cₐ-C_{b})cycloalkyl as indicated herein, wherein the (Cₐ-C_{b})cycloalkyl group is appended to the parent molecular moiety through an oxygen atom, e.g. cyclopentyloxy or cyclobutyloxy.

The term “(Cₐ-C_{b})cycloalkyl(Cₐ-C_{b})alkyl” is intended to indicate an (Cₐ-C_{b})alkyl group as defined herein substituted with one or more (Cₐ-C_{b})cycloalkyl as defined herein, suitably the (Cₐ-C_{b})alkyl group is substituted with one (Cₐ-C_{b})cycloalkyl group.

The term “halo(Cₐ-C_{b})alkyl” is intended to indicate an (Cₐ-C_{b})alkyl group as defined herein substituted with one or more halogen atoms as defined herein, e.g. fluoro or chloro, such as difluoromethyl or trifluoromethyl.

The term “halogen” is intended to indicate a substituent from the 7^{th} main group of the periodic table, such as fluoro, chloro and bromo.

The term “5- or 6-membered heteroaryl” is intended to indicate radicals of monocyclic heteroaromatic rings comprising 5- or 6-membered ring which contains from 1-5 carbon atoms and from 1-4 heteroatoms selected from oxygen, sulphur and nitrogen; such as 2-5 carbon atoms and 1-3 heteroatoms, such as 3-5 carbon atoms and 1-2 heteroatoms, such as 4-5 carbon atoms and 1-2 heteroatoms selected from oxygen, sulphur and nitrogen, such as furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, tetrazolyl, thiadiazolyl, thiazolyl and triazolyl. The term “5- or 6-membered heteroaryl” includes compounds wherein a ring member is a C(O) or carbonyl group.

The term “5-membered heteroaryl” is intended to indicate radicals of 5-membered monocyclic heteroaromatic ring which contains from 1-4 carbon atoms and from 1-4 heteroatoms selected from oxygen, sulphur and nitrogen; such as 2-4 carbon atoms and 1-3 heteroatoms, such as 3-4 carbon atoms and 1-2 heteroatoms, such as 4 carbon atoms and 1 heteroatom selected from oxygen, sulphur and nitrogen; such as furanyl, imidazolyl, isothiazolyl, isoxazolyl, oxadiazolyl, oxazolyl, pyrazolyl, pyrrolyl, tetrazolyl, thiadiazolyl, thiazolyl and triazolyl. The term “5-membered heteroaryl” includes compounds wherein a ring member is a C(O) or carbonyl group.

The term “9- or 10-membered bicyclic heteroaryl” is intended to indicate fused bicyclic heteroaromatic radicals comprising 9- or 10-carbon or heteroatoms, which for example contain from 3-9 carbon atoms and 1-7 heteroatoms selected from oxygen, sulphur and nitrogen, such as 1-5 heteroatoms and 5-9 carbon atoms, such as 1-3 heteroatoms and 7-9 carbon atoms, such as 1-2 heteroatoms and 8-9 carbon atoms, such as 1 heteroatom and 8 carbon atoms, such as 1 heteroatom and 9 carbon atoms, such as 2 heteroatom and 7 carbon atoms, such as 2 heteroatom and 8 carbon atoms. Said bicyclic heteroaromatic radicals comprise a 5- or 6-membered heteroaromatic ring fused to phenyl and a 5- or 6-membered heteroaromatic ring fused to another 5- or 6-membered heteroaromatic ring, as defined herein. The heteroaryl radical may be connected to the parent molecular moiety through a carbon atom or a nitrogen atom contained anywhere within the heteroaryl group. Representative examples of 9- or 10-membered bicyclic heteroaryl include, but are not limited to azaindolyl, benzofuranyl, benzothiophenyl, benzimidazolyl, benzooxazolyl, benzothiazolyl, benzothienyl, cinnolyl, imidazopyridinyl, imidazopyrimidinyl, indazolyl, indolyl, isobenzofuranyl, isoquinolyl, quinolyl, pyrrolopyrimidinyl, thienopyridinyl. pyrrolo[2,3]pyridinyl, pyrrolo[2,3]pyridinyl, pyrazolo[1,5]pyridinyl, pyrazolo[1,5]pyridazinyl, imidazo[1,2]pyrimidinyl, pyrrolo[2,3-c]pyridinyl, pyrrolo[2,3-b]pyridinyl, pyrazolo[1,5-a]pyridinyl, pyrazolo[1,5-b]pyridazinyl, imidazo[1,2-a]pyrimidinyl.

The term (5- or 6-membered heteroaryl)-(Cₐ-C_{b})alkyl is intended to indicate a 5- or 6-membered heteroaryl appended to the parent molecular moiety through a (Cₐ-C_{b})alkyl group, as defined herein.

The term “(a-b) membered heterocycloalkyl” is intended to indicate a cycloalkane radical as described herein, including polycyclic radicals such as bicyclic or tricyclic radicals, including spirocyclic radicals, wherein one or more carbon atoms of said cycloalkane radical are replaced by heteroatoms, i.e. the a-b membered heterocycloalkyl comprise from a to b carbon- or hetero-atoms. Such a-b membered heterocycloalkyl could comprise for example 2-9 carbon atoms and 1-6 heteroatoms selected from O, N, or S, such as 3-8 carbon atoms and 1-4 heteroatoms, such as 3-7 carbon atoms and 1-3 heteroatoms, such as 3-6 carbon atoms and 1-2 heteroatom. The heterocycloalkyl radical may be connected to the parent molecular moiety through a carbon atom or a nitrogen atom contained anywhere within the heterocycloalkyl group. Representative examples of heterocycloalkyl groups include, but are not limited to azepanyl, azetidinyl, aziridinyl, dioxolanyl, dioxolyl, imidazolidinyl, morpholinyl, oxetanyl, piperazinyl, piperidinyl, pyrrolidinyl, tetrahydrofuranyl, tetrahydropyranyl, thietanyl, 2,6-diazaspiro[3.3]heptane, 2,6-diazaspiro[3.3]heptane, 2,6-diazaspiro[3.3]heptanyl, 2,5-diazabicyclo[2.2.1]heptanyl, 2-oxa-5-aza-[2.2.1]heptanyl, 2-oxa-8-azaspiro[3.5]nonanyl, 2-oxa-7-azaspiro[3.5]nonanyl, 2-oxa-8-azaspiro[3.5]nonanyl, 6-oxa-2-azaspiro[3.3]heptanyl, 2-oxa-7-azaspiro[3,4]octanyl, and 1, 3, 3a, 4, 6, 6a-hexahydrofuro[3,4-c]pyrrolyl. The term includes compounds wherein a ring member of said “(a-b) membered heterocycloalkyl” is a C(O) or carbonyl group and S(O) group.

The term “(a-b membered heterocycloalkyl)-(C_{c}-C_{d})alkyl” is intended to indicate a a-b membered heterocycloalkyl radical appended to the parent molecular moiety through an (C_{c}-C_{d})alkyl group, as defined herein.

The term “hydrocarbon radical” is intended to indicate a radical containing only hydrogen and carbon atoms, it may contain one or more double and/or triple carbon-carbon bonds, and it may comprise cyclic moieties in combination with branched or linear moieties. Said hydrocarbon comprises 1-6 carbon atoms, e.g. 1-5, e.g. 1-4, e.g. 1-3, e.g. 1-2 carbon atoms. The term includes alkyl and cycloalkyl as indicated herein.

The term “hydroxy(Cₐ-C_{b})alkyl” is intended to indicate an (Cₐ-C_{b})alkyl group as defined above substituted with one or more hydroxy, e.g. hydroxymethyl, hydroxyethyl, hydroxypropyl.

The term “oxo” is intended to indicate an oxygen atom which is connected to the parent molecular moiety via a double bond (═O).

The term “phenyl-(Cₐ-C_{b})alkyl” is intended to indicate a phenyl group appended to appended to the parent molecular moiety through an (Cₐ-C_{b})alkyl group, as defined herein.

When two or more of the above defined or similar terms are used in combination, such as cycloalkylalkyl or phenyl-(Cₐ-C_{b})alkyl and the like, it is to be understood that the first mentioned radical is a substituent on the latter mentioned radical, where the point of attachment to the parent molecular moiety is on the latter radical.

The group C(O) is intended to represent a carbonyl group (C═O).

If substituents are described as being independently selected from a group, each substituent is selected independent of the other. Each substituent may therefore be identical or different from the other substituent(s).

The term “optionally substituted” means “unsubstituted or substituted”, and therefore the general formulas described herein encompasses compounds containing the specified optional substituent(s) as well as compounds that do not contain the optional substituent(s).

As used herein whenever a molecular drawing of a substituent contains an arrow—the arrow indicates the bond attaching the substituent to the rest of the molecule.

The term “pharmaceutically acceptable salt” is intended to indicate salts prepared by reacting a compound of formula I, which comprise a basic moiety, with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxyethanesulfonic acid, toluenesulfonic, sulfamic or fumaric acid. Pharmaceutically acceptable salts of compounds of formula I comprising an acidic moiety may also be prepared by reaction with a suitable base such as sodium hydroxide, potassium hydroxide, magnesium hydroxide, calcium hydroxide, zinc hydroxide, barium hydroxide, ammonia or the like, or suitable non-toxic amines, such as lower alkylamines (such as diethylamine, tetraalkylammonium hydroxide), hydroxy-lower alkylamines (such as diethanolamine, 2-(diethylamino)-ethanol, ethanolamine, triethanolamine, tromethamine, deanol), cycloalkylamines, ethylene diamine, or benzylamines, (such as benethamine and benzathine), betaine, choline hydroxide, N-methyl-glucamine, hydrabamine, 1H-imidazole, 4-(2-hydroxyethyl)-morpholine, piperazine, 1-(2-hydroxyethyl)-pyrrolidine, L-arginine or L-lysine. Further examples of pharmaceutical acceptable salts are listed in Berge, S. M.; J. Pharm. Sci.; (1977), 66(1), 1-19, and Stahl, P. H. and in Wermuth, C. G, Handbook of Pharmaceutical Salts, Properties, Selection and Use, 2^{nd} Edition, Wiley-VCH, 2011 both of which are incorporated herein by reference.

For example when R₂contains -L-PO(OH)₂ the phosphoric acid group may form a salt with a monovalent cation M⁺ or divalent cation Q²⁺ to form a group selected from -L-PO(OH)O⁻·M⁺, -L-PO(OH)O⁻·1/2Q²⁺ L-PO(O⁻)₂·2M⁺, and -L-PO(O⁻)₂·Q²⁺.

The term ‘monovalent cation’ is intended to indicate monovalent cations such as alkali metal ions, such as for example sodium (Na⁺), potassium (K⁺) or lithium (Li⁺), or ammonium ions, such as for example NH₄₊, dialkylammonium (NH₂((C₁-C₄)alkyl)₂)⁺, trialkylammonium (NH((C₁-C₄)alkyl)₃)⁺, or tetraalkylammonium (N((C₁-C₄)alkyl)₄)⁺, alkylammonium (H₃N(C₁-C₄)alkyl)⁺ or hydroxyalkylammonium (H₃N-hydroxy(C₁-C₄)alkyl)⁺, the protonated forms of L-arginine, L-lysine or the protonated forms of any pharmaceutically acceptable bases such as those mentioned above.

The term ‘divalent cation’ is intended to indicate divalent cations such as alkaline earth metal ions such as calcium (Ca²⁺), Magnesium (Mg²⁺), barium (Ba²⁺), or Zinc (Zn²⁺).

The term ‘prodrug’ is intended to indicate compounds which are drug-precursors which, upon administration, are converted to the parent drug in vivo by enzymatic and/or chemical reactions. Generally, the pro-drug is less biologically active than its parent drug. The prodrug may have improved physical-chemical properties compared to the parent drug, such as improved aqueous solubility, thereby facilitating the absorption and consequently the bioavailability of the parent compound upon administration.

The term ‘parent drug’ or ‘parent compound’ is intended to indicate the biologically active compound which is released from the prodrug via enzymatic and/or chemical processes following administration of the prodrug. The parent drug is frequently the starting material for the preparation of the corresponding prodrug.

Examples of prodrugs according to the invention are prodrugs that are attached to a nitrogen or oxygen of the parent molecule.

For example when the parent molecule contains a 5-membered heteroaryl containing nitrogen substituted with hydrogen as a ring atom said hydrogen may be replaced with a substituent selected from -L-PO(OH)₂, wherein L is selected from the group consisting of a bond or —CHR_{g}O— and R_{g} is selected from hydrogen and (C₁-C₆)alkyl to form a prodrug.

5-membered heteroaryls such as pyrrole, imidazole, pyrazole, triazole and tetrazole when attached to the reminder of the molecule via a carbon ring atom are moieties that may contain a nitrogen ring atom substituted by hydrogen.

The term “solvate” is intended to indicate a species formed by interaction between a compound, e.g. a compound of formula I, and a solvent, e.g. alcohol, glycerol or water, wherein said species are in a crystalline form. When water is the solvent, said species is referred to as a hydrate.

The term “or pharmaceutically acceptable salts, hydrates and solvates thereof” includes compound of formula (I) and hydrates or solvates thereof, and pharmaceutically acceptable salts of the compounds of formula(I) as well as hydrates or solvates thereof.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

All references, including publications, patent applications and patents, cited herein are hereby incorporated by reference in their entirety and to the same extent as if each reference were individually and specifically indicated to be incorporated by reference, regardless of any separately provided incorporation of particular documents made elsewhere herein.

In one embodiment, the invention relates to a compound of formula (I) or formula (Ia),

wherein X, Y, Z and V are each independently selected from N, CH and C(R₄); R₄ is selected from (C₁-C₆)alkyl, (C₁-C₆)alkoxy, amino, hydroxy and halogen, wherein said (C₁-C₆)alkyl and (C₁-C₆)alkoxy is optionally substituted with one of more substituents independently selected from halogen;

Q is C(R₅), or N;

R₁ is selected from —CHR₆R₇, and wherein R₆ and R₇ each independently represents hydrogen, phenyl, cyclopropyl, cyclobutyl, cyclopentyl, cyclohexyl, cycloheptyl, cyclopropylmethyl, cyclobutylmethyl, methyl or ethyl, wherein said phenyl, cyclopropyl, cyclobutyl, cyclopentyl, cyclohexyl, cyclopropylmethyl, cyclobutylmethyl, methyl or ethyl, is optionally substituted with one or more substituents independently selected from halogen, cyano, and (C₁-C₄)alkyl; with the proviso that at least one of R₆ and R₇ is different from hydrogen;

R₂ is selected from the group consisting of 5- or 6-membered heteroaryl, wherein said 5-or 6-membered heteroaryl is optionally substituted with one or more substituents independently selected from Rₐ, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

L is selected from the group consisting of a bond or —CHR_{g}O—,

R_{g} is independently selected from hydrogen and (C₁-C₆)alkyl;

R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b};

R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy,

or pharmaceutically acceptable salts, hydrates and solvates thereof.

In another embodiment, the invention relates to a compound of formula (I) or formula (Ia),

wherein X, Y, Z and V are each independently selected from N, CH and C(R₄); R₄ is selected from (C₁-C₆)alkyl, (C₁-C₆)alkoxy, amino, hydroxy and halogen, wherein said (C₁-C₆)alkyl and (C₁-C₆)alkoxy is optionally substituted with one of more substituents independently selected from halogen;

Q is C(R₅), or N;

R₁ is selected from —CHR₆R₇, and wherein R₆ and R₇ each independently is selected from (C₃-C₇)cycloalkyl and (C₃-C₇)cycloalkyl-(C₁-C₆)alkyl, wherein said (C₃-C₇)cycloalkyl and (C₃-C₇)cycloalkyl-(C₁-C₆)alkyl, is optionally substituted with one or more substituents independently selected from halogen, cyano, and (C₁-C₄)alkyl;

R₂ is selected from the group consisting of 5- or 6-membered heteroaryl, wherein said 5-or 6-membered heteroaryl is optionally substituted with one or more substituents independently selected from Rₐ, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

L is selected from the group consisting of a bond or —CHR_{g}O—,

R_{g} is independently selected from hydrogen and (C₁-C₆)alkyl;

R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b};

R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy,

or pharmaceutically acceptable salts, hydrates and solvates thereof.

In one embodiment, the invention relates to a compound of formula (I) or formula (Ia) above, wherein R₁ is selected from —CHR₆R₇, and wherein R₆ and R₇ each independently represents (C₃-C₇)cycloalkyl, wherein said (C₃-C₇)cycloalkyl is optionally substituted with one or more substituents independently selected from halogen, cyano, and (C₁-C₄)alkyl.

In another embodiment, the invention relates to a compound of formula (I) or formula (Ia), wherein X, Y, Z and V are each independently selected from N, CH and C(R₄); R₄ is selected from (C₁-C₆)alkyl, (C₁-C₆)alkoxy, amino, hydroxy and halogen, wherein said (C₁-C₆)alkyl and (C₁-C₆)alkoxy is optionally substituted with one of more substituents independently selected from halogen;

Q is C(R₅), or N;

R₁ is selected from cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl or spiro[2.5]octanyl, wherein said cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl or spiro[2.5]octanyl is optionally substituted with one or more substituents independently selected from deuterium, halogen, cyano, (C₁-C₄)alkyl and halo(C₁-C₄)alkyl;

R₂ is selected from the group consisting of 5- or 6-membered heteroaryl, wherein said 5-or 6-membered heteroaryl is optionally substituted with one or more substituents independently selected from Rₐ, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

L is selected from the group consisting of a bond or —CHR_{g}O—,

R_{g} is independently selected from hydrogen and (C₁-C₆)alkyl;

R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b};

R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy,

or pharmaceutically acceptable salts, hydrates and solvates thereof.

In one embodiment, the invention relates to a compound of formula (I) or formula (Ia) above wherein R₁ is cyclohexyl optionally substituted with one or more (C₁-C₄)alkyl.

In another embodiment, the invention relates to a compound of formula (I) or formula (Ia),

wherein X, Y, Z and V are each independently selected from N, CH and C(R₄); R₄ is selected from (C₁-C₆)alkyl, (C₁-C₆)alkoxy, amino, hydroxy and halogen, wherein said (C₁-C₆)alkyl and (C₁-C₆)alkoxy is optionally substituted with one of more substituents independently selected from halogen;

Q is C(R₅), or N;

R₁ is selected from G, wherein G is

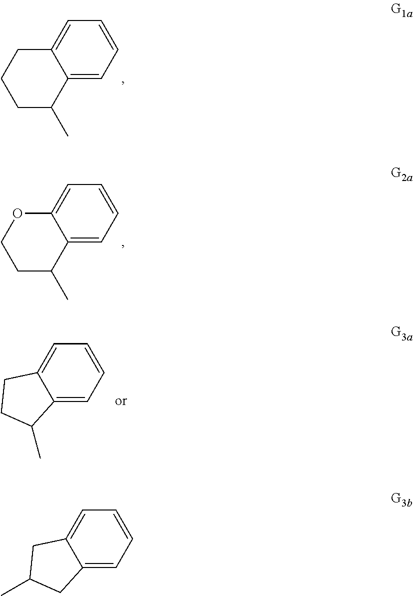

wherein said G is optionally substituted with one or more substituents independently selected from deuterium, halogen, cyano, (C₁-C₄)alkyl and halo(C₁-C₄)alkyl.

R₂ is selected from the group consisting of 5- or 6-membered heteroaryl, wherein said 5-or 6-membered heteroaryl is optionally substituted with one or more substituents independently selected from Rₐ, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

L is selected from the group consisting of a bond or —CHR_{g}O—,

R_{g} is independently selected from hydrogen and (C₁-C₆)alkyl;

R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b};

R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy,

or pharmaceutically acceptable salts, hydrates and solvates thereof.

In one embodiment, the invention relates to a compound of formula (I) or formula (Ia), wherein R₂ is selected from pyrazolyl and imidazolyl, wherein said pyrazolyl or imidazolyl is optionally substituted with one or more substituents independently selected from (C₁-C₆)alkyl.

In one specific embodiment, the invention relates to a compound of formula (I) or formula (Ia), wherein R₂ is 3,5-di(C₁-C₆)alkyl-pyrazol-4-yl or 3,5-di(C₁-C₆)alkyl-imidazol-4-yl.

In one embodiment, the invention relates to a compound of formula (I) or formula (Ia), wherein R₂ is selected from 3,5-di(C₁-C₆)alkyl-pyrazol-4-yl or 3,5-di(C₁-C₆)alkyl-imidazol-4-yl, wherein said 3,5-di(C₁-C₆)alkyl-pyrazol-4-yl or 3,5-di(C₁-C₆)alkyl-imidazol-4-yl contain a nitrogen ring atom substituted by a substituent selected from -L-PO(OH)₂.

In one embodiment, the invention relates to a compound of formula (I) or formula (Ia),

wherein R₃ is selected from —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b}; and

Q is C(R₅) and R₅ is selected from hydrogen, halogen, hydroxy, (C₁-C₆)alkyl, (C₃-C₇)cycloalkyl, (C₃-C₇)cycloalkoxy, (C₁-C₆)alkoxy; or Q is N.

In one embodiment, the invention relates to a compound of formula (I) or formula (Ia), wherein R₃ is selected from —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b}; and

Q is C(R₅) and R₅ is selected from hydrogen, halogen, hydroxy, (C₁-C₆)alkyl, (C₃-C₇)cycloalkyl, (C₃-C₇)cycloalkoxy, (C₁-C₆)alkoxy.

In one embodiment, the invention relates to a compound of formula (I) or formula (Ia), wherein R₃ is selected from —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b}; and

Q is N.

In one embodiment, the invention relates to a compound of formula (I) or formula (Ia),

wherein X, Y, Z and V are independently selected from CH and C(R₄),

X is N and Y, Z and V are independently selected from CH and C(R₄),

Y is N and X, Z and V are independently selected from CH and C(R₄),

X and Y are N and V and Z are independently selected from CH and C(R₄),

Y and Z are N and X and V are independently selected from CH and C(R₄),

X and Z are N and Y and V are independently selected from CH and C(R₄), or

Y and V are N and X and Z are independently selected from CH and C(R₄).

In one embodiment, the invention relates to a compound of formula (I) or formula (Ia), wherein X is N, Y is C(R₄) and V and Z are CH.

In one embodiment, the invention relates to a compound of formula (I) or formula (Ia), wherein

wherein X, Y, Z and V is CH;

Q is N;

R₁ is selected from the group consisting of —CHR₆R₇,

R₆ and R₇ each independently represents (C₃-C₇)cycloalkyl, or (C₃-C₇)cycloalkyl-(C₁-C₆)alkyl;

R₂ is 3,5-di-((C₁-C₆)alkyl)-1H-pyrazol-4-yl;

R₃ is 4H-1,2,4-triazol-3-yl wherein said 4H-1,2,4-triazol-3-yl (is optionally substituted with one or more substituents independently selected from R_{b};

R_{b} is (C₁-C₆)alkyl, or (C₃-C₇)cycloalkyl, wherein said (C₁-C₆)alkyl, or (C₃-C₇)cycloalkyl independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy,

or pharmaceutically acceptable salts, hydrates and solvates thereof.

In one or more embodiments of the present invention, the compounds of general formula I have an (EC₅₀) value in an IL-8 release assay of less than 1 micromolar, or of less than 100 nanomolar.

The compounds of formula I may be obtained in crystalline form either directly by concentration from an organic solvent or by crystallisation or recrystallisation from an organic solvent or mixture of said solvent and a cosolvent that may be organic or inorganic, such as water. The crystals may be isolated in essentially solvent-free form or as a solvate, such as a hydrate. The invention covers all crystalline forms, such as polymorphs and pseudopolymorphs, and also mixtures thereof.

Compounds of formula I comprise asymmetrically substituted (chiral) carbon atoms which give rise to the existence of isomeric forms, e.g. enantiomers and possibly diastereomers. The present invention relates to all such isomers, either in optically pure form or as mixtures thereof (e.g. racemic mixtures or partially purified optical mixtures). Pure stereoisomeric forms of the compounds and the intermediates of this invention may be obtained by the application of procedures known in the art. The various isomeric forms may be separated by physical separation methods such as selective crystallization and chromatographic techniques, e.g. high pressure liquid chromatography using chiral stationary phases. Enantiomers may be separated from each other by selective crystallization of their diastereomeric salts which may be formed with optically active amines, or with optically active acids. Optically purified compounds may subsequently be liberated from said purified diastereomeric salts. Enantiomers may also be resolved by the formation of diastereomeric derivatives. Alternatively, enantiomers may be separated by chromatographic techniques using chiral stationary phases. Pure stereoisomeric forms may also be derived from the corresponding pure stereoisomeric forms of the appropriate starting materials, provided that the reaction occur stereoselectively or stereospecifically. Preferably, if a specific stereoisomer is desired, said compound will be synthesized by stereoselective or stereospecific methods of preparation. These methods will advantageously employ chiral pure starting materials.

Furthermore, when a double bond or a fully or partially saturated ring system is present in the molecule geometric isomers may be formed. Any geometric isomer, as separated, pure or partially purified geometric isomers or mixtures thereof are included within the scope of the invention.

The formula (I), (Ia) and (Ib) includes all tautomers and all mixtures thereof.

In the compounds of general Formula I, the atoms may exhibit their natural isotopic abundances, or one or more of the atoms may be artificially enriched in a particular isotope having the same atomic number, but an atomic mass or mass number different from the atomic mass or mass number found in nature. The present invention includes all suitable isotopic variations of the compounds of general Formula I. For example, different isotopic forms of hydrogen include ¹H, ²H and ³H, different isotopic forms of carbon include ¹²C, ¹³C and ¹⁴C and different isotopic forms of nitrogen include ¹⁴N and ¹⁵N. Enriching for deuterium (²H) may for example increase in-vivo half-life or reduce dosage regiments, or may provide a compound useful as a standard for characterization of biological samples. Isotopically enriched compounds within general formula I can be prepared by conventional techniques well known to a person skilled in the art or by processes analogous to those described in the general procedures and examples herein using appropriate isotopically enriched reagents and/or intermediates.

Some compounds have lower aqueous solubility which may affect the absorption and consequently the bioavailability of the compounds. Such compounds may advantageously be administered in the form of prodrugs improving the aqueous solubility of the parent compound. Such prodrugs which, upon administration, are converted to their parent compounds may be less active in vitro compared to their parent compounds, but because of the improved aqueous solubility, facilitating the absorption and consequently the bioavailability of the parent compounds upon administration, such prodrugs have improved in vivo activity compared to their parent compounds.

Prodrugs of the compounds of formula (I) form part of the invention claimed.

Solvates and hydrates form part of the invention claimed.

The compounds of the present invention may be useful for preventing, treating or ameliorating any of the following diseases: psoriasis, ankylosing spondylitis, spondyloarthritis or psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer.

In an embodiment the invention relates to the use of a compound of general formula (I) as defined above, in the manufacture of a medicament for the prophylaxis, treatment or amelioration of any of the following diseases: psoriasis, ankylosing spondylitis, spondyloarthritis or psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer.

In an embodiment the invention relates to the use of a compound of general formula (I) as defined above, in the manufacture of a medicament for the prophylaxis, treatment or amelioration of autoimmune diseases, such as psoriasis, ankylosing spondylitis, spondyloarthritis or psoriatic arthritis.

In an embodiment the invention relates to a method of preventing, treating or ameliorating autoimmune diseases, such as psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer, the method comprising administering to a person suffering from at least one of said diseases an effective amount of one or more compounds according to general formula (I), optionally together with a pharmaceutically acceptable carrier or one or more excipients, optionally in combination with other therapeutically active compounds.

In an embodiment the invention relates to a method of preventing, treating or ameliorating autoimmune diseases, such as psoriasis, ankylosing spondylitis, spondyloarthritis or psoriatic arthritis, the method comprising administering to a person suffering from at least one of said diseases an effective amount of one or more compounds according to general formula (I), optionally together with a pharmaceutically acceptable carrier or one or more excipients, optionally in combination with other therapeutically active compounds.

Besides being useful for human treatment, the compounds of the present invention may also be useful for veterinary treatment of animals including mammals such as horses, cattle, sheep, pigs, dogs, and cats.

Pharmaceutical Compositions of the Invention

For use in therapy, compounds of the present invention are typically in the form of a pharmaceutical composition. The invention therefore relates to a pharmaceutical composition comprising a compound of formula I, optionally together with one or more other therapeutically active compound(s), together with a pharmaceutically acceptable excipient, vehicle or carrier(s). The excipient must be “acceptable” in the sense of being compatible with the other ingredients of the composition and not deleterious to the recipient thereof.

Conveniently, the active ingredient comprises from 0.0001-99.9% by weight of the formulation.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.001 mg and 1000 mg, preferably between 0.01 mg and 300 mg of a compound of formula I.

A suitable dosage of the compound of the invention will depend, inter alia, on the age and condition of the patient, the severity of the disease to be treated and other factors well known to the practising physician. The compound may be administered either orally, parenterally, topically, transdermally or intradermally and other routes according to different dosing schedules, e.g. daily, weekly or with monthly intervals. In general, a single dose will be in the range from 0.001 to 400 mg/kg body weight.

If the treatment involves administration of another therapeutically active compound it is recommended to consult Goodman & Gilman's The Pharmacological Basis of Therapeutics, 9^{th} Ed., J. G. Hardman and L. E. Limbird (Eds.), McGraw-Hill 1995, for useful dosages of said compounds.

The administration of a compound of the present invention with one or more other active compounds may be either concomitantly or sequentially.

The formulations include e.g. those in a form suitable for oral, rectal, parenteral transdermal, intradermal, ophthalmic, topical, nasal, sublingual or buccal administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005. All methods include the step of bringing the active ingredient into association with the carrier, which constitutes one or more accessory ingredients. In general, the formulations are prepared by uniformly and intimately bringing the active ingredient into association with a liquid carrier, semisolid carrier or a finely divided solid carrier or combinations of these, and then, if necessary, shaping the product into the desired formulation.

Formulations of the present invention suitable for oral and buccal administration may be in the form of discrete units as capsules, sachets, tablets, chewing gum or lozenges, each containing a predetermined amount of the active ingredient.

A tablet may be made by compressing, moulding or freeze drying the active ingredient optionally with one or more accessory ingredients. Compressed tablets may be prepared by compressing, in a suitable machine, the active ingredient(s) in a free-flowing form; for example with a lubricant; a disintegrating agent or a dispersing agent. Moulded tablets may be made by moulding, in a suitable machine, a mixture of the powdered active ingredient and suitable carrier. Freeze dried tablets may be formed in a freeze-dryer from a solution of the drug substance.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Liposomal formulations are also suitable for parenteral administration.

Transdermal formulations may be in the form of a plaster, patch, microneedles, liposomal or nanoparticulate delivery systems or other cutaneous formulations applied to the skin.

Formulations suitable for ophthalmic administration may be in the form of a sterile aqueous preparation of the active ingredients. Liposomal formulations or biodegradable polymer systems may also be used to present the active ingredient for ophthalmic administration.

Formulations suitable for topical, such as dermal, intradermal or ophthalmic administration include liquid or semi-solid preparations, solutions or suspensions.

Formulations suitable for nasal or buccal administration include powder, self-propelling and spray formulations, such as aerosols and atomisers.

Methods of Preparation

The compounds of the present invention can be prepared in a number of ways well known to those skilled in the art of synthesis. The compounds of the invention could for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used.

The compounds of the present invention or any intermediate could be purified, if required, using standard methods well known to a synthetic organist chemist, e.g. methods described in “Purification of Laboratory Chemicals”, 6^{th} ed. 2009, W. Amarego and C. Chai, Butterworth-Heinemann.

Starting materials are either known or commercially available compounds, or may be prepared by routine synthetic methods well known to a person skilled in the art.

Unless otherwise noted, reagents and solvents were used as received from commercial suppliers. The organic solvents used were usually anhydrous. The solvent ratios indicated refer to vol:vol unless otherwise noted. Thin layer chromatography was performed using Merck 6OF254 silica-gel TLC plates. Visualisation of TLC plates was performed using UV light (254 nm) or by an appropriate staining technique.

Proton nuclear magnetic resonance spectra were obtained at the stated frequencies in the solvents indicated. Tetramethylsilane was used as an internal standard for proton spectra. The value of a multiplet, either defined doublet (d), triplet (t), quartet (q) or (m) at the approximate midpoint is given unless a range is quoted. (br) indicates a broad peak, whilst (s) indicates a singlet.

Mass spectra were obtained using the following methods. LCMS Method 1 was used, unless otherwise stated.

LCMS Method 1:

Column: Acquity UPLC HSS T3 1.8 μm; 2.1×50 mm

Flow: 0.7 mL/min

Column temp: 30° C.

Mobile phases: A: 10 mM Ammonium acetate+0.1% formic acid, B: 100% Acetonitrile+0.1% formic acid

UV: 240-400 nm

Injection volume: 1 μl

Gradient:

| Time (min) | A % | B % |
|---|---|---|
| 0.0 | 99% | 1% |
| 0.5 | 94% | 6% |
| 1.0 | 94% | 6% |
| 2.6 | 5% | 95% |
| 3.8 | 5% | 95% |
| 3.81 | 99% | 1% |
| 4.8 | 99% | 1% |

UPLC (inlet method): XEV Metode 1 CM

MS-method: Pos_50_1000 or Neg_50_1000

Instruments: Waters Acquity UPLC, Waters XEVO G2-XS QTof, Waters PDA (Photodiode Array)

LCMS Method 2:

Mass spectra were obtained on a Waters Quattro micro API/Waters SQD2/Waters Quattro Premier Spectrometer using electrospray ionization and atmospheric-pressure chemical ionization with the column and solvents indicated.

LCMS Method 3:

Column: Waters Acquity UPLC HSS T3 1.8 μm, 2.1×50 mm.

Column temperature: 60° C.

UV: PDA 210-400 nm.

Injection volume: 2 μl.

Eluents: A: 10 mM Ammonium acetate with 0.1% formic acid, B: 100% Acetonitrile with 0.1% formic acid.

Gradient:

| Time (min) | A % | B % | Flow (mL/min) |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray switching between positive and negative ionisation.

Instruments: Waters ACQUITY UPLC, Waters SQD, Waters PDA (Photodiode array)

LCMS Method 4:

Column: Waters ACQUITY UPLC BEH 1.7 μm, 2.1×50 mm.

Column temperature: 60° C.

UV: PDA 210-400 nm.

Injection volume: 2 μl.

Eluents: A: 10 mM Ammonium Bicarbonate, B: 100% Acetonitrile

Gradient:

| Time (min) | % A | % B | Flow (mL/min) |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray positive or negative ionisation.

Instruments: Waters ACQUITY UPLC, Waters QDa (MS detector), Waters PDA (Photodiode Array)

Basic Preparative HPLC Conditions:

Column: XBridge Prep C18 5 μm OBD, 19×150 mm

Eluents: Ammonium formate (50 mM)/acetonitrile, 10-100% acetonitrile

Flow: 30 mL/min

Acidic Preparative HPLC Conditions:

Column: XTerra® RP-18 5 μm OBD, 19×150 mm

Eluents: 0.1% formic acid in water/acetonitrile, 10-100% acetonitrile

Flow: 30 mL/min

The following abbreviations have been used throughout:

AcOH acetic acid

Boc tert-butoxycarbonyl

BOP (benzotriazol-1-yloxy)tris(dimethylamino)phosphonium hexafluorophosphate

BuLi butyllithium

CBz benzyloxycarbonyl

CDI carbonyldiimidazole

DCC dicyclohexylcarbodiimide

DCM dichloromethane

DIPEA diisopropylethylamine

DMF N,N-dimethylformamide

DMF·DMA N,N-dimethylformamide dimethyl acetal

DMSO dimethylsulfoxide

dppf 1,1′-bis(diphenylphosphino)ferrocene

EDC N-(3-Dimethylaminopropyl)-N′-ethylcarbodiimide

EtOAc ethyl acetate

EtOH ethanol

HATU 1-[bis(dimethylamino)methylene]-1H-1,2,3-triazolo[4,5-b]pyridinium 3-oxide hexafluorophosphate

HBTU N,N,N′,N′-tetramethyl-O-(1H-benzotriazol-1-yl)uronium hexafluorophosphate

HOBt hydroxybenzotriazole

HPLC high-performance liquid chromatography

IPA isopropyl alcohol

LCMS liquid chromatography-mass spectrometry

Me methyl

MeCN acetontitrile

MeOH methanol

MHz megahertz

NBS N-bromosuccinimide

NCS N-chlorosuccinimide

NMP N-methyl-2-pyrrolidinone

NMR nuclear magnetic resonance

ppm parts per million

Prep. preparation

Prep. HPLC preparative HPLC

PTFE polytetrafluoroethane

PyBOP (benzotriazol-1-yloxy)tripyrrolidinophosphonium hexafluorophosphate

SEM 2-(trimethylsilyl)ethoxymethyl

SFC supercritical fluid chromatography

SM starting material

TBME tert-butyl methyl ether

TEA triethylamine

TFA trifluoroacetic acid

THF tetrahydrofuran

TMS trimethylsilyl

TLC thin layer chromatography

T3P propanephosphonic acid anhydride

General Methods

Compounds of the invention may be prepared according to the following non-limiting general methods and examples:

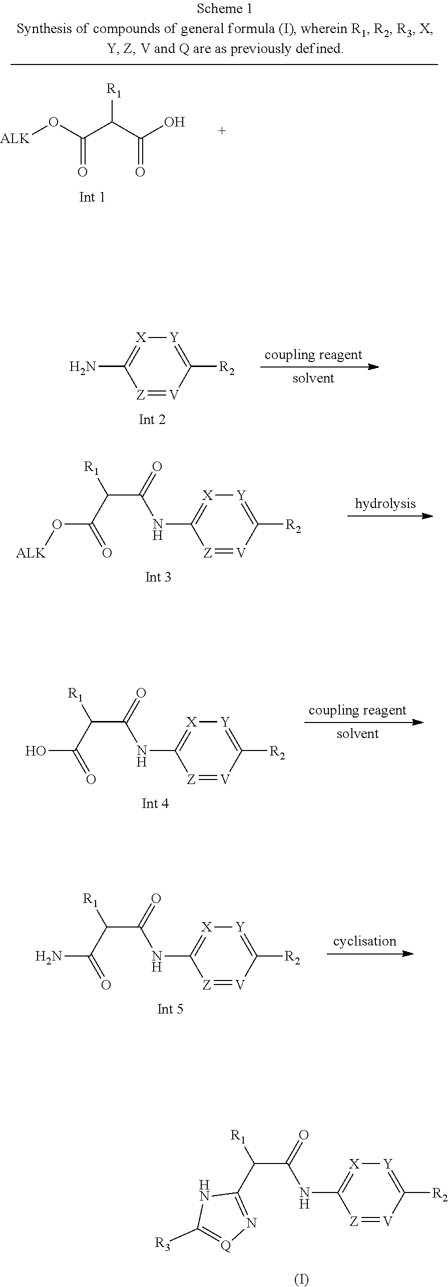

Compounds of general formula (I) can be prepared, as shown in Scheme 1. Compounds of general formula (Int 1), which are either commercially available or are synthesized, are coupled with amines of general formula (Int 2), which are either commercially available or synthesised, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 3). Compounds of general formula (Int 3) can be hydrolysed with an appropriate base such as LiOH, KOH or NaOH in a suitable solvent such as MeOH, EtOH or THF, to give compounds of general formula (Int 4). Compounds of general formula (Int 4) are coupled with ammonium chloride, in the presence of a coupling reagent such as HATU, HBTU, CDI, T3P, PyBOP, BOP, DCC or EDC and HOBt and in most of the cases in the presence of a base, such as DIPEA or triethylamine, in a suitable solvents, such as DMF or acetonitrile to form compounds of general formula (Int 5). Compounds of general formula (Int 5) can be reacted with commercially available DMF·DMA, then condensed with hydrazine in a suitable solvent such as AcOH to give compounds of general formula (I). Where the compounds of general formula (I) contain protecting groups, those protecting groups can be removed by methods known to those skilled in the art. Racemic compounds of general formula (I) can be separated by chiral SFC, to give the S-enantiomers of compounds of general formula (I).

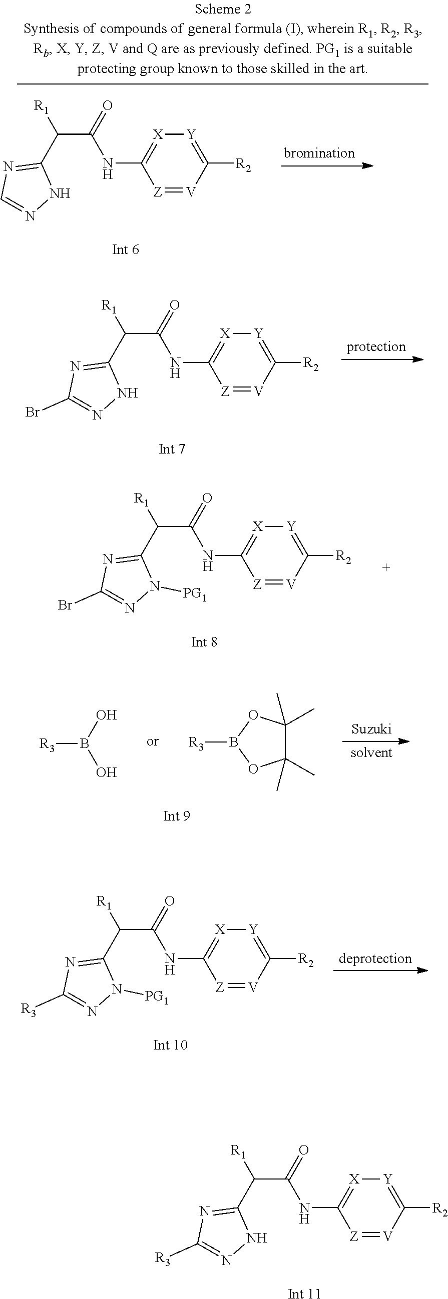

Compounds of general formula (Int 11) can be prepared, as shown in Scheme 2. Those skilled in the art will appreciate that some of the embodiments of R₃ can undergo literature precedented transformations before deprotection, for example to give compounds of general formula (Int 10). Compounds of general formula (Int 6) can react with benzyltrimethylammonium tribromide in a suitable solvent such as DCM in the presence of an aqueous base solution, such as NaOH, to give compounds of general formula (Int 7).

Introduction of a suitable protecting group (PG₁) can be accomplished by methods known to those skilled in the art, to give compounds of general formula (Int 8). Compounds of general formula (Int 10) can be prepared by reaction with a commercially available or synthesised R₃-boronic compound (Int 9), such as (2-fluorophenyl)boronic acid, in the presence of a catalyst such as tetrakis(triphenylphosphine)palladium or [1,1′-bis(diphenylphosphino)ferrocene]palladium (II) dichloride PdCl₂(dppf), in the presence of an aqueous base, such as K₂CO₃ or Na₂CO₃, in a suitable solvent, such as DMF or 1,4-dioxane to form compounds of formula (Int 10). The protecting groups (PG₁ and where applicable, PG) on compounds of formula (Int 10) can be removed by methods known to those skilled in the art to give compounds of formula (Int 11). Racemic compounds of general formula (Int 10) and (Int 11) can be separated by chiral SFC, to give the S-enantiomers of compounds of general formula (Int 10) and (Int 11).

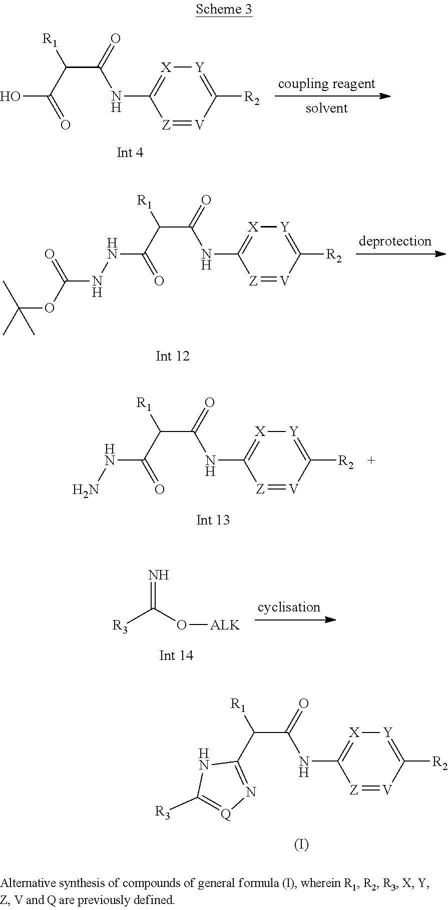

Compounds of general formula (I) can also be prepared, as shown in Scheme 3. Compounds of general formula (Int 4), are coupled with commercially available tert-butyl N-aminocarbamate, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 12). Selective removal of the Boc protecting group can be achieved employing methods known to those skilled in the art, to give compounds of general formula (Int 13). Compounds of general formula (Int 13) can be reacted with suitable carboximidates of general formula (Int 14) that are either commercially available or can be synthesized, in a suitable solvent such as MeOH, IPA or DMF, and optionally in the presence of a base such as TEA, to give compounds of general formula (I), where Q=N. Racemic compounds of general formula (I) can be separated by chiral SFC, to give the S-enantiomers of compounds of general formula (I).

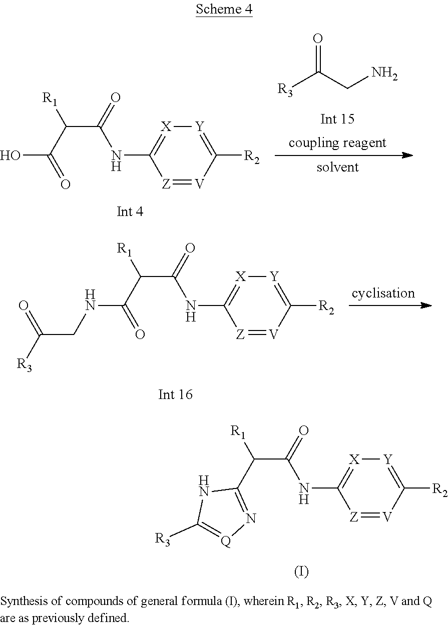

Compounds of general formula (I) can also be prepared, as shown in Scheme 4. Compounds of general formula (Int 4), are coupled with amines of general formula (Int 15), that are either commercially available or synthesized, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of general formula (Int 16). Compounds of general formula (I), where Q=CH, can be prepared from the reaction of compounds of general formula (Int 16) with a suitable ammonium salt, such as ammonium acetate or ammonium chloride, in a suitable solvent such as EtOH, toluene or AcOH, optionally in the presence of a base such as TEA, at elevated temperature such as 80-160° C. Racemic compounds of general formula (I) can be separated by chiral SFC, to give the S-enantiomers of compounds of general formula (I).

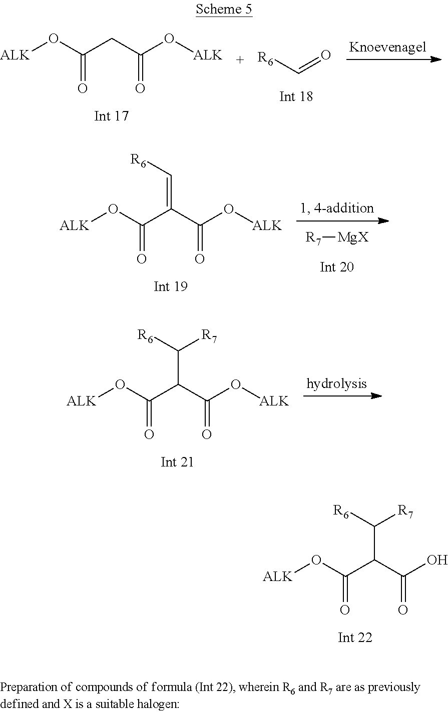

Compounds of general formula (Int 22) can be synthesised as outlined in Scheme 5. Compounds of general formula (Int 17), which are either commercially available or can be synthesised, can be condensed with compounds of general formula (Int 18) in the presence of AcOH and a base such as piperidine, in a suitable solvent such as EtOH or toluene, to give compounds of general formula (Int 19). Compounds of general formula (Int 21) can be prepared by the addition of Grignard reagents (Int 20), which are either commercially available or can be synthesised, in the presence of copper iodide, to compounds of general formula (Int 19) in suitable solvents such as THF at a temperature of −78° C. Compounds of general formula (Int 21) can be hydrolysed with an appropriate base such as LiOH, KOH or NaOH in a suitable solvent such as MeOH, EtOH or THF, to give compounds of general formula (Int 22).

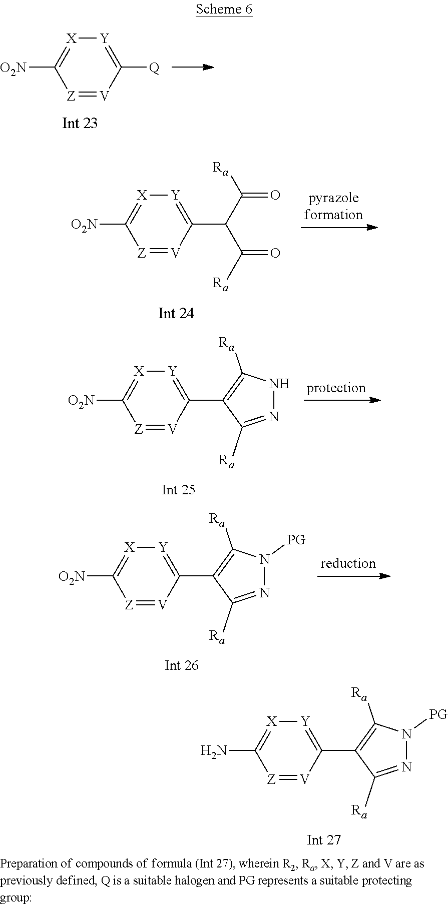

Compounds of general formula (Int 27) can be synthesised as outlined in Scheme 6. Compounds of general formula (Int 23), which are either commercially available or can be synthesised according to methods known to those skilled in the art, can be reacted with an appropriate 1,3-diketone to give compounds of general formula (Int 24). For example, when Q=Br or I a compound of general formula (Int 23) can be reacted with an appropriate 1,3-diketone in the presence of CuI, proline and a suitable base, such as K₂CO₃, in an appropriate solvent, such as DMSO, at an elevated temperature, for example 70-100° C. Alternatively, when Q=F, a compound of general formula (Int 23) can be reacted with an appropriate 1,3-diketone in the presence of a suitable base, such as K₂CO₃ or Cs₂CO₃, in a suitable solvent, such as DMF or DMSO, at an elevated temperature, for example 50-100° C. Compounds of general formula (Int 25) can be prepared by reaction of compounds of general formula (Int 24) with hydrazine hydrate in a suitable solvent, such as EtOH, at an appropriate temperature, for example from room temperature to 80° C. The compounds of formula (Int 26) can be synthesised by methods known to those skilled in the art using, for example, SEM chloride or Boc anhydride, to give compounds of general formula (Int 26). Reduction of the nitro group in compounds of general formula (Int 26) can be carried out by many methods known to those skilled in the art to give anilines of general formula (Int 27). For example, by catalytic hydrogenation, using a suitable catalyst, such as Pd on carbon, in a suitable solvent, such as EtOAc, MeOH or EtOH, under a suitable pressure of hydrogen.

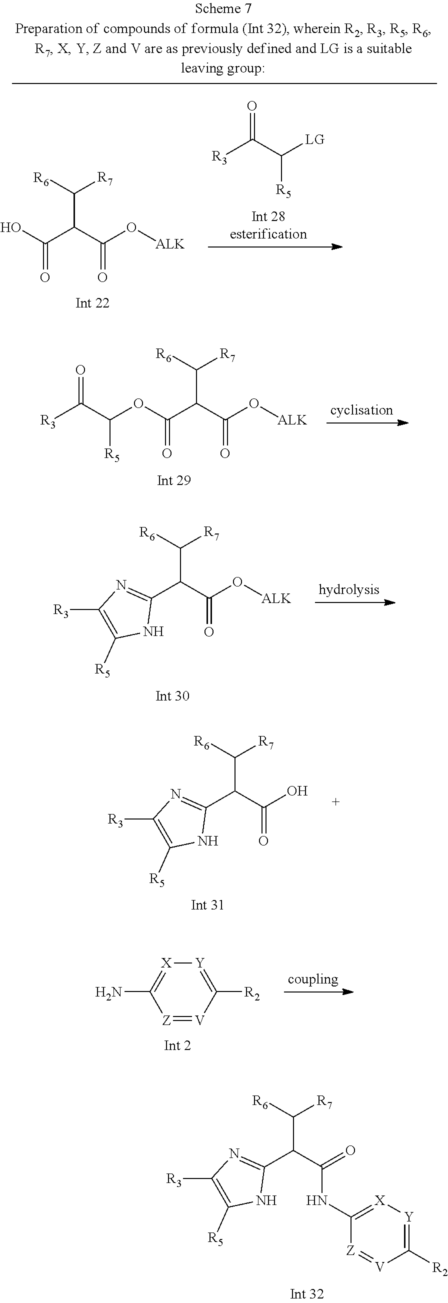

Compounds of general formula (Int 32) can be synthesised as outlined in Scheme 7. Compounds of general formula (Int 22) can be reacted with compounds of general formula (Int 28) that are either commercially available or can be synthesised, in the presence of a suitable base such as K₂CO₃ or Cs₂CO₃, in a suitable solvent such as DMSO or DMF to give compounds of general formula (Int 29). Compounds of general formula (Int 30) can be prepared from the reaction of compounds of general formula (Int 29) with a suitable ammonium salt, such as ammonium acetate or ammonium chloride, in a suitable solvent such as EtOH, toluene or AcOH, optionally in the presence of a base such as TEA, at elevated temperature such as 80-160° C. Compounds of general formula (Int 30) can be hydrolysed with an appropriate base such as LiOH, KOH or NaOH in a suitable solvent such as MeOH, EtOH or THF, to give compounds of general formula (Int 31). Compounds of general formula (Int 31) are coupled with amines of general formula (Int 2), which are either commercially available or synthesised, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 32). Racemic compounds of general formula (Int 32) can be separated by chiral SFC, to give the S-enantiomers of compounds of general formula (Int 32).

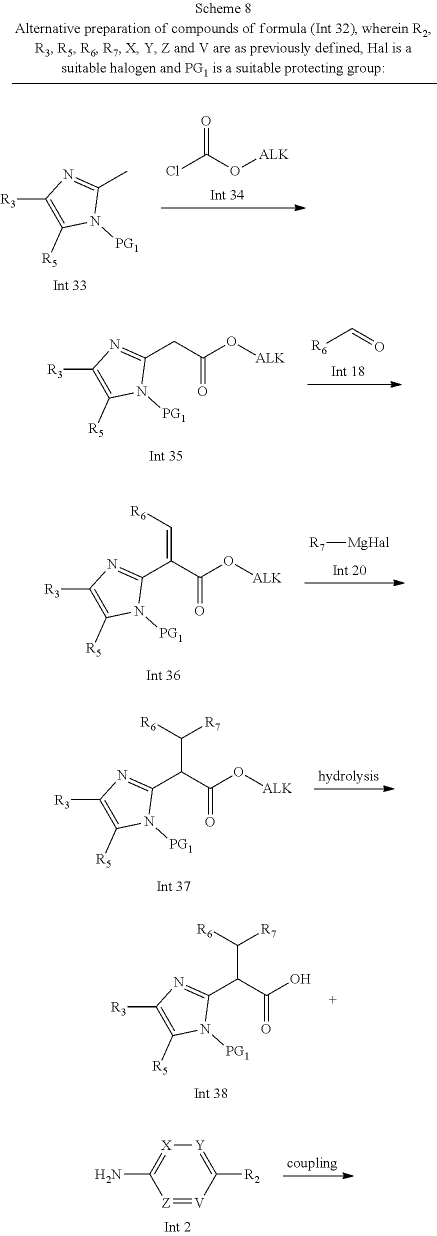

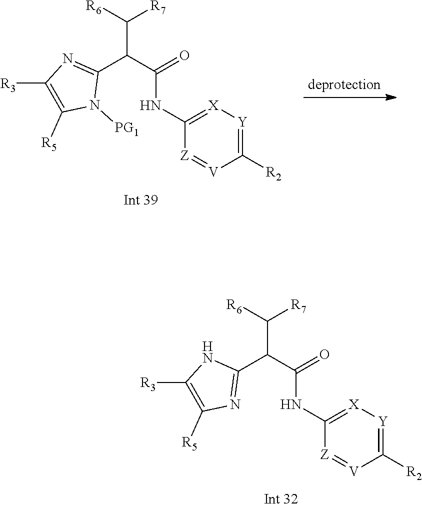

Compounds of general formula (Int 32) can be synthesised as outlined in Scheme 8. Compounds of general formula (Int 33) that are either commercially available or synthesised, can react with compounds of general formula (Int 34) that are either commercially available or synthesised, in the presence of a base such as TEA in a suitable solvent such as MeCN to give compounds of formula (Int 35). Compounds of general formula (Int 35) can be condensed with compounds of general formula (Int 18) in the presence of AcOH and a base such as piperidine, in a suitable solvent such as EtOH or toluene, to give compounds of general formula (Int 36). Compounds of general formula (Int 37) can be prepared by the addition of Grignard reagents (Int 20), which are either commercially available or can be synthesised, in the presence of copper iodide, to compounds of general formula (Int 36) in suitable solvents such as THF at a temperature of −78° C. Compounds of general formula (Int 37) can be hydrolysed with an appropriate base such as LiOH, KOH or NaOH in a suitable solvent such as MeOH, EtOH or THF, to give compounds of general formula (Int 38). Compounds of general formula (Int 38) are coupled with amines of general formula (Int 2), which are either commercially available or synthesised, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 39). Where the compounds of general formula (Int 39) contain protecting groups, those protecting groups can be removed or selectively removed by methods known to those skilled in the art. Racemic compounds of general formula (Int 39) and (Int 32) can be separated by chiral SFC, to give the S-enantiomers of compounds of general formula (Int 39) and (Int 32).

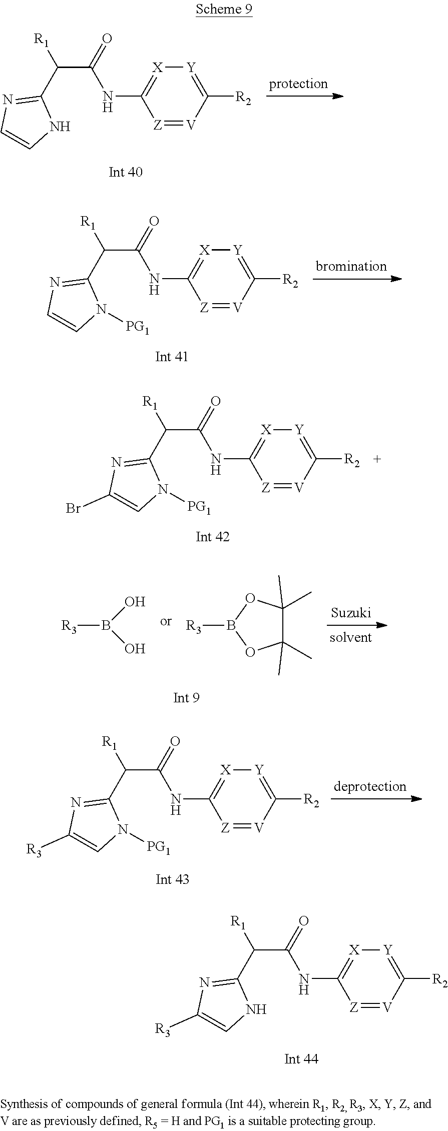

Compounds of general formula (Int 44) can be prepared, as shown in Scheme 9 from compounds of general formula (Int 40). Introduction of a suitable protecting group (PG₁) can be accomplished by methods known to those skilled in the art, to give compounds of general formula (Int 41). Compounds of general formula (Int 41) can react with benzyltrimethylammonium tribromide or NBS in a suitable solvent such as DCM, with or without the presence of an aqueous base solution, such as NaOH, to give compounds of general formula (Int 42). Compounds of general formula (Int 42) can be reacted with a commercially available or synthesised R₃-boronic compounds (Int 9), in the presence of a catalyst such as [1,1′-bis(diphenylphosphino)ferrocene]palladium (II) dichloride PdCl₂(dppf), in the presence of an aqueous base, such as K₂CO₃ or Na₂CO₃, in a suitable solvent, such as DMF or 1,4-dioxane to form compounds of formula (Int 43). The protecting groups PG₁ can be removed by methods known to those skilled in the art to give compounds of formula (Int 44). Racemic compounds of general formula (Int 43) and (Int 44) can be separated by chiral SFC, to give the S-enantiomers of compounds of general formula (Int 43) and (Int 44).

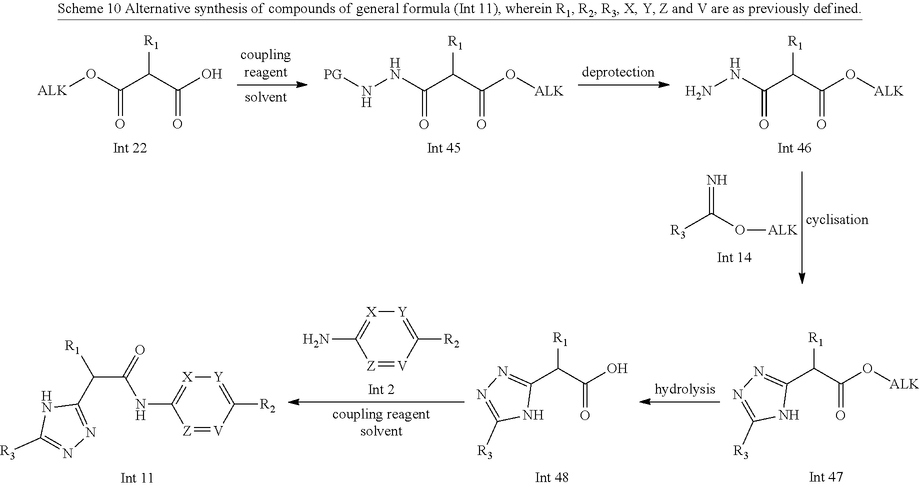

Compounds of general formula (Int 11) can also be prepared, as shown in Scheme 10. Compounds of general formula (Int 22), are coupled with suitable, commercially available protected hydrazines, such as tert-butyl N-aminocarbamate, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 45). Selective removal of the protecting group can be achieved employing methods known to those skilled in the art, to give compounds of general formula (Int 46). Compounds of general formula (Int 46) can be reacted with suitable carboximidates of general formula (Int 14) that are either commercially available or can be synthesized, in a suitable solvent such as MeOH, IPA or DMF, and optionally in the presence of a base such as TEA, to give compounds of general formula (Int 47). Compounds of general formula (Int 47) can be hydrolysed with an appropriate base such as LiOH, KOH or NaOH in a suitable solvent such as MeOH, EtOH or THF, to give compounds of general formula (Int 48). Compounds of general formula (Int 48) are coupled with amines of general formula (Int 2), which are either commercially available or synthesised, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 11). Racemic compounds of general formula (Int 11) can be separated by chiral SFC, to give the S-enantiomers of compounds of general formula (Int 11).

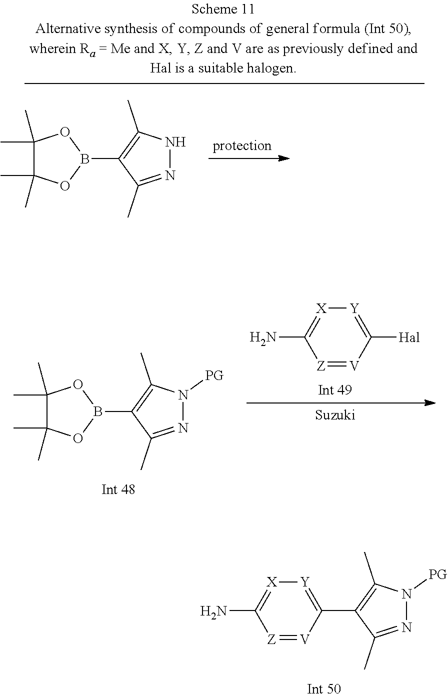

Compounds of general formula (Int 50) can be prepared, as shown in Scheme 11. Commercially available 3,5-dimethyl-4-(4,4,5,5-tetramethyl-1,3,2-dioxaborolan-2-yl)-1H-pyrazole can be protected by methods known to those skilled in the art using, for example, SEM chloride in the presence of a suitable base such as K₂CO₃ in a suitable solvent such as DMF. Compounds of general formula (Int 48) can be reacted with compounds of general formula (Int 49) in the presence of palladiium source such as [1,1′-bis(diphenylphosphino)ferrocene]palladium(II) dichloride PdCl₂(dppf), or bis(triphenylphosphine)palladium(II) dichloride, PdCl₂(PPh₃)₂, in the presence of an aqueous base, such as K₂CO₃ or Na₂CO₃, in a suitable solvent, such as DMF or toluene to give compounds of general formula (Int 50).

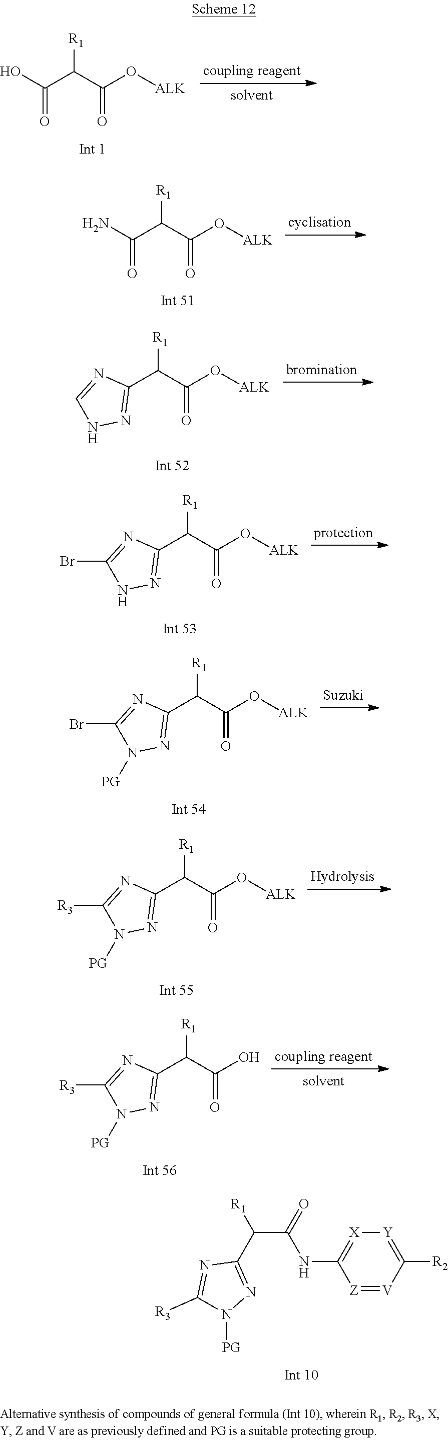

Compounds of general formula (Int 10) can be prepared, as shown in Scheme 12. Compounds of general formula (Int 1) can be reacted with a suitable source of ammonia, such as ammonium chloride, in the presence of a coupling reagent such as HATU, HBTU, CDI, T3P, PyBOP, BOP, DCC or EDC and HOBt and in most of the cases in the presence of a base, such as DIPEA or triethylamine, in a suitable solvents, such as DMF or acetonitrile to form compounds of general formula (Int 51). Compounds of general formula (Int 51) can be reacted with commercially available DMF·DMA, then condensed with hydrazine in a suitable solvent such as AcOH to give compounds of general formula (Int 52). Compounds of general formula (Int 52) can be reacted with a suitable bromine source, such as NBS, in a suitable solvent such as DCM, DMF or MeCN, to give compounds of general formula (Int 53). Introduction of a suitable protecting group (PG) can be accomplished by methods known to those skilled in the art, to give compounds of general formula (Int 54). Compounds of general formula (Int 55) can be prepared by reaction of compounds of general formula (Int 54) with a commercially available or synthesised R₃-boronic compound (Int 9), in the presence of a catalyst such as tetrakis(triphenylphosphine)palladium or [1,1′-bis(diphenylphosphino)ferrocene]palladium (II) dichloride PdCl₂(dppf), in the presence of an aqueous base, such as K₂CO₃ or Na₂CO₃, in a suitable solvent, such as DMF or 1,4-dioxane. Compounds of general formula (Int 55) can be hydrolysed with an appropriate base such as LiOH, KOH or NaOH in a suitable solvent such as MeOH, EtOH or THF, to give compounds of general formula (Int 56). Compounds of general formula (Int 56), can be coupled with amines of general formula (Int 2), which are either commercially available or synthesised, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 10).

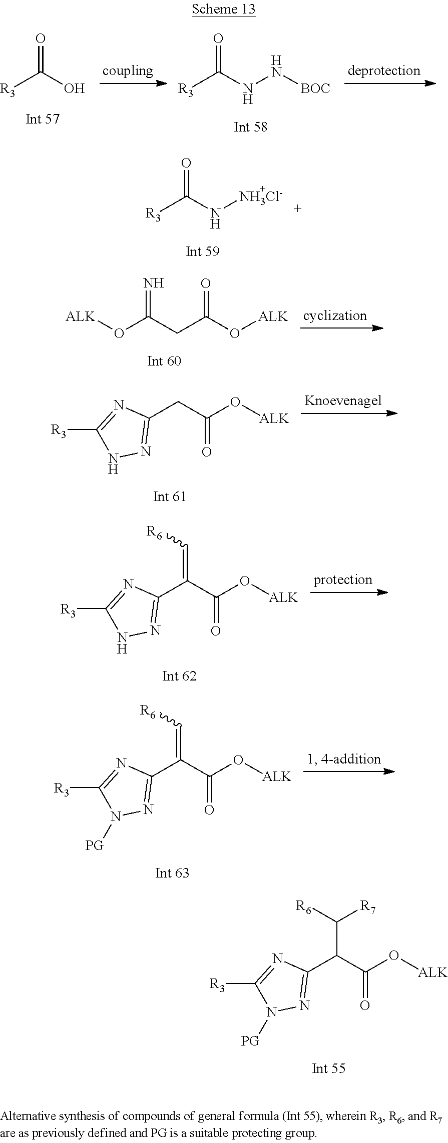

Compounds of general formula (Int 55) can be prepared, as shown in Scheme 13. Compounds of general formula (Int 57), which are commercially available or synthesised by methods known to those skilled in the art, are coupled with commercially available tert-butyl N-aminocarbamate, in the presence of a coupling reagent such as T3P, CDI, DCC, HATU, HBTU or EDC, and in the majority of cases, in the presence of a base, such as DIPEA or TEA, in a suitable solvent, such as DMF or acetonitrile to form compounds of formula (Int 58). Removal of the Boc protecting group can be achieved employing methods known to those skilled in the art, to give compounds of general formula (Int 59). Compounds of general formula (Int 59) can be reacted with suitable carboximidates of formula (Int 60) that are either commercially available or can be synthesized, in a suitable solvent such as MeOH, MeCN, IPA or DMF, and optionally in the presence of a base such as TEA, optionally in the presence of AcOH, to give compounds of general formula (Int 61), Compounds of general formula (Int 61), can be condensed with compounds of general formula (Int 18) optionally in the presence of AcOH and a base such as piperidine, in a suitable solvent such as EtOH or toluene, to give compounds of general formula (Int 62). Compounds of general formula (Int 62) can be protected with a suitable protecting group using methods known to those skilled in the art, for example can be synthesised by methods known to those skilled in the art using, for example, by reaction with SEM chloride in the presence of a suitable base such as K₂CO₃ in a suitable solvent such as DMF, to give compounds of general formula (Int 63). Compounds of general formula (Int 55) can be prepared by the addition of Grignard reagents (Int 20), which are either commercially available or can be synthesised, in the presence of copper iodide, to compounds of general formula (Int 63) in suitable solvents such as THF at a suitable temperature, such as at −78° C.

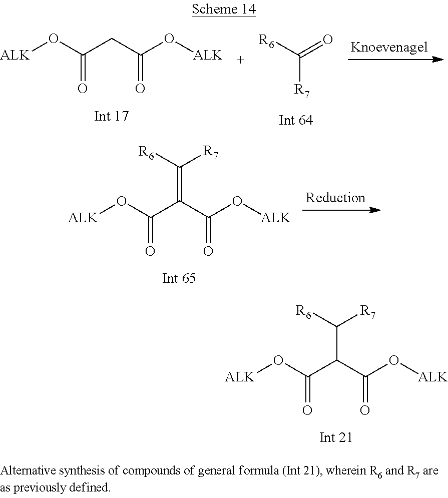

Compounds of general formula (Int 21) can be synthesised as outlined in Scheme 14. Compounds of general formula (Int 17), which are either commercially available or can be synthesised, can be condensed with compounds of general formula (Int 64) in the presence of AcOH and a base such as piperidine, in a suitable solvent such as EtOH or toluene, or alternatively in the presence of a Lewis acid, such as TiCl₄, in a suitable solvent mixture, such as a mixture of DCM and THF, to give compounds of general formula (Int 65). Compounds of general formula (Int 21) can be prepared by reduction of the compounds of general formula (Int 65), for example by treatment with hydrogen gas in the presence of a suitable catalyst, such as Pd on carbon, in a suitable solvent, such as MeOH, EtOH or EtOAc,

### PREPARATIONS AND EXAMPLES

### Preparations

### Preparation 1: diethyl 2-(cyclopropylmethylene)propanedioate

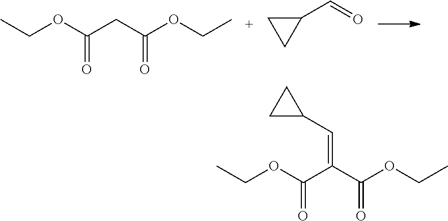

Cyclopropanecarbaldehyde (5.00 g, 71.3 mmol) was added to a solution of diethyl propanedioate (10.8 mL, 71.3 mmol), piperidine (0-141 mL, 1.43 mmol) and acetic acid (0.082 mL, 1.43 mmol) in EtOH (71.3 mL) and the reaction mixture was stirred at 100° C. for 18 hours. The reaction mixture was concentrated in vacuo. The obtained crude compound was purified by silica column chromatography (230-400 mesh), eluting with EtOAc (0-50%) in heptane, to afford the title compound (13.8 g, 80% yield). 1H NMR (400 MHz, CDCl₃) δ 6.35 (d, J=11.3 Hz, 1H), 4.32 (q, J=7.1 Hz, 2H), 4.26-4.16 (m, 2H), 1.97 (dtt, J=12.2, 8.2, 4.4 Hz, 1H), 1.34 (t, J=7.1 Hz, 3H), 1.31-1.25 (m, 3H), 1.16-1.05 (m, 2H), 0.80-0.70 (m, 2H).

### Preparation 2: diethyl 2-(dicyclopropylmethyl)propanedioate

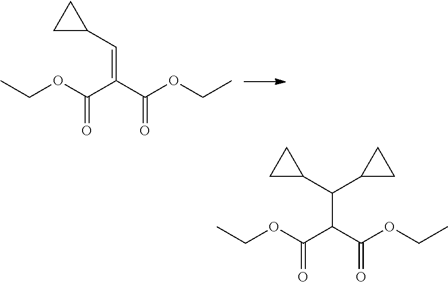 [SMILES: CCOC(=O)C(=CC1CC1)C(=O)OCC ; CCOC(=O)C(C(=O)OCC)C(C1CC1)C1CC1]

Bromo(cyclopropyl)magnesium (1M solution in 2-MeTHF, 63.8 mL, 63.8 mmol) was added dropwise to a pre-stirred solution of CuI (6.08 g, 31.9 mmol) in anhydrous THF (145 mL) at 0° C. On complete addition the reaction mixture was stirred at 0° C. for 30 minutes, then cooled to −78° C. A solution of the compound of Preparation 1 (7.0 g, 29.02 mmol) in anhydrous THF (145 mL) was then added dropwise and the reaction mixture was stirred at −78° C. for 30 minutes, then allowed to warm to room temperature overnight. The reaction mixture was quenched with NH₄Cl (aqueous solution) and diluted with water (100 mL). The mixture was then extracted with Et₂O (2×200 mL). The combined organic extracts were washed with brine solution, dried over MgSO₄, filtered and concentrated in vacuo. The obtained crude compound was purified by silica column chromatography (230-400 mesh), eluting with EtOAc (0-50%) in heptane, to afford the title compound (6.34 g, 86% yield). 1H NMR (400 MHz, CDCl₃) δ 4.29-4.15 (m, 4H), 3.52 (d, J=6.7 Hz, 1H), 1.35-1.20 (m, 6H), 1.02-0.77 (m, 3H), 0.58-0.44 (m, 2H), 0.42-0.31 (m, 2H), 0.29-0.10 (m, 4H).

### Preparation 3: 2-(dicyclopropylmethyl)-3-ethoxy-3-oxo-propanoic acid

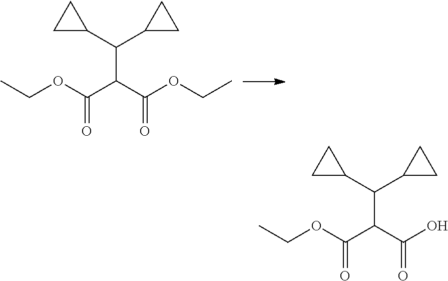 [SMILES: CCOC(=O)C(C(=O)OCC)C(C1CC1)C1CC1 ; CCOC(=O)C(C(=O)O)C(C1CC1)C1CC1]

A solution of KOH (220 mg, 3.8 mmol) in water (0.67 mL) was added dropwise to a solution of the compound of Preparation 2 (850 mg, 3.3 mmol) in EtOH (2 mL) at 0° C., over 20 minutes. On complete addition the reaction mixture was stirred at 0° C. for 30 minutes then at room temperature for 18 hours. The reaction mixture was diluted with water (5 mL) and acidified with HCl (5M aq. solution) to pH 1-2. The reaction mixture was then extracted with Et₂O (2×5 mL). The combined organic extracts were washed with brine solution (2 mL), dried over MgSO₄, filtered and concentrated in vacuo, to afford the title compound (525 mg, 69% yield). 1H NMR (400 MHz, CDCl₃) δ 4.26 (q, J=7.1 Hz, 2H), 3.58 (d, J=4.7 Hz, 1H), 1.32 (t, J=7.1 Hz, 3H), 0.97-0.78 (m, 3H), 0.62-0.38 (m, 4H), 0.32-0.15 (m, 4H).

### Preparation 4: 3-(4-Nitrophenyl)pentane-2,4-dione

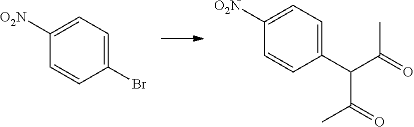 [SMILES: O=[N+]([O-])c1ccc(Br)cc1 ; CC(=O)C(C(C)=O)c1ccc([N+](=O)[O-])cc1]

1-Iodo-4-nitro-benzene (5.0 g, 20.1 mmol), pentane-2,4-dione (4.01 g, 40.2 mmol) and K₂CO₃ (6.92 g, 50.2 mmol) were taken in dry DMSO (100 mL) and purged with argon gas for 15 min. CuI (0.381 g, 2.00 mmol) was added, followed by (S)-Proline (0.461 g, 4.01 mmol). The resulting reaction mixture was stirred at 70° C. for 16 hours. The reaction mixture was diluted with water (100 mL) and extracted with EtOAc (2×100 mL). The combined organic layers were washed with water (100 mL) and brine (100 mL), dried (Na₂SO₄) and concentrated under reduced pressure. The residue was purified by column chromatography (silica, eluting with 5% EtOAc in petroleum ether) to give the title compound (1.8 g, 40% yield) as a yellow solid. 1H NMR (400 MHz, CDCl₃) δ 16.76 (s, 1H), 8.27 (d, J=8.8 Hz, 2H), 7.39 (d, J=8.8 Hz, 2H), 1.90 (s, 6H); LCMS (Method 2) (ES): m/z=220 [M−H]⁻, RT=1.98 min (Column: X-Bridge BEH (4.6 mm×50 mm, 2.5 μM), Mobile phase: A: 5 mM Ammonium bicarbonate, B: MeCN).

### Preparation 5: 3,5-Dimethyl-4-(4-nitrophenyl)-1H-pyrazole

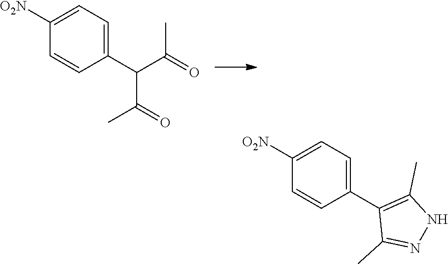 [SMILES: CC(=O)C(C(C)=O)c1ccc([N+](=O)[O-])cc1 ; Cc1n[nH]c(C)c1-c1ccc([N+](=O)[O-])cc1]

Hydrazine hydrate (56.5 mL, 1130 mmol) was added to a stirred solution of the compound of Preparation 4 (50 g, 226 mmol) in EtOH (1 L) at room temperature. The reaction mixture was then heated at 70° C. for 6 hours. The reaction mixture was concentrated under reduced pressure and the residue was diluted with water (1 L) and stirred at room temperature for 20 minutes. The precipitate was filtered, washed with cold water (300 mL) and hexane (300 mL). The solid was dried to give the title compound (35 g, 71% yield) as a yellow solid. 1H NMR (400 MHz, DMSO-d₆) δ 12.55 (br s, 1H), 8.26-8.23 (d, J=8.8 Hz, 2H), 7.59-7.57 (d, J=9.2 Hz, 2H), 2.29 (s, 3H), 2.23 (s, 3H); LCMS (Method 2) (ES): m/z=218 [M+H]⁺, RT=5.62 min (Column: X-Bridge BEH (4.6 mm×50 mm, 2.5 μM), Mobile phase: A: 5 mM Ammonium bicarbonate, B: MeCN).

### Preparation 6: 2-[[3,5-dimethyl-4-(4-nitrophenyl)pyrazol-1-yl]methoxy]ethyl-trimethyl-silane

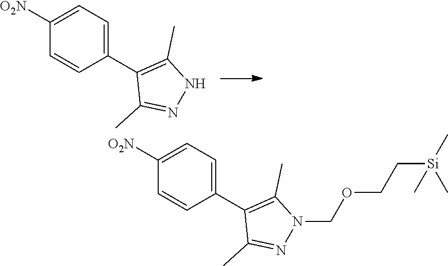

To a stirred solution of the compound of Preparation 5 (20.0 g, 92.2 mmol) in DMF (400 mL) was added 60% NaH (7.37 g, 184 mmol) at 0° C. The reaction mixture was stirred at 0° C. for 10 min then SEM chloride (24.4 mL, 138 mmol) was added. The resulting reaction mixture was stirred at room temperature for 1 h. The reaction mixture was poured into ice cold water (1 L) and extracted with EtOAc (2×500 mL). The combined organic layer was washed with ice cold water (3×250 mL) and brine (300 mL), dried over anhydrous Na₂SO₄, filtered and concentrated under reduced pressure. The obtained crude compound was purified by silica gel (100-200 mesh) column chromatography (20% EtOAc in Hexane as eluent) to give the title compound (23 g, 72% yield) as a pale brown viscous liquid. 1H NMR (300 MHz, DMSO-d6) δ 8.39-8.19 (m, 2H), 7.69-7.40 (m, 2H), 5.39 (s, 2H), 3.64-3.53 (m, 2H), 2.33 (s, 3H), 2.21 (s, 3H), 0.92-0.76 (m, 2H), −0.03 (s, 9H); LCMS (Method 3) (ES): m/z 384.3 [M+H]⁺, RT=0.95 min.

### Preparation 7: 4-[3,5-dimethyl-1-(2-trimethylsilylethoxymethyl)pyrazol-4-yl]aniline

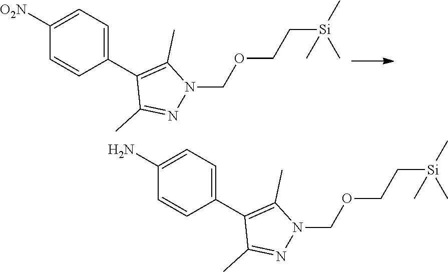

10% Pd/C (188 mg) was added to a solution of the compound of Preparation 6 (1.88 g, 5.41 mmol) in MeOH (30 mL) and placed under hydrogen at atmospheric pressure. After 1 hour the catalyst was filtered off, washing with MeOH, and the filtrate was concentrated in vacuo to give the title compound (1.67 g, 97%) as a colourless solid. 1H NMR (300 MHz, DMSO-d6) δ 6.96-6.85 (m, 2H), 6.65-6.57 (m, 2H), 5.30 (s, 2H), 5.03 (s, 2H), 3.59-3.48 (m, 2H), 2.20 (s, 3H), 2.08 (s, 3H), 0.83 (dd, J=8.4, 7.4 Hz, 2H), −0.04 (s, 9H); LCMS (Method 3) (ES): m/z 318.4 [M+H]⁺, RT=0.80 min.

### Preparation 8: ethyl 2-(dicyclopropylmethyl)-3-[4-[3,5-dimethyl-1-(2-trimethylsilylethoxymethyl)pyrazol-4-yl]anilino]-3-oxo-propanoate

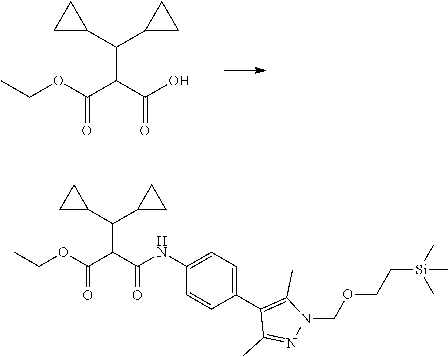 [SMILES: CCOC(=O)C(C(=O)O)C(C1CC1)C1CC1 ; CCOC(=O)C(C(=O)Nc1ccc(-c2c(C)nn(COCC[Si](C)(C)C)c2C)cc1)C(C1CC1)C1CC1]

HATU (891 mg, 2.34 mmol) was added to a solution of the compound of Preparation 3 (530 mg, 2.34 mmol), the compound of Preparation 7 (744 mg, 2.34 mmol) and DIPEA (0.816 mL, 4.68 mmol) in DMF (5 mL) and stirred at room temperature for 1 hour. The reaction mixture was diluted with Et₂O (25 mL) and washed successively with water, NaHSO₄ (10% aq. solution) NaHCO₃ (aq. solution) and brine (5 mL of each). The washed organic layer was dried over MgSO₄, filtered and concentrated in vacuo to afford the title compound (1.103 g, 89% yield). LCMS (Method 3) (ES): m/z 526.4 [M+H]⁺, RT=1.00 min.

### Preparation 9: 2-(dicyclopropylmethyl)-3-[4-[3,5-dimethyl-1-(2-trimethylsilylethoxymethyl)pyrazol-4-yl]anilino]-3-oxo-propanoic acid

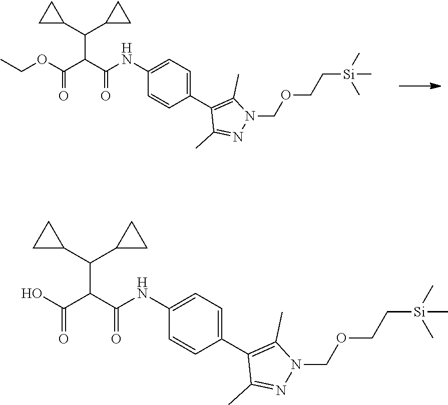 [SMILES: CCOC(=O)C(C(=O)Nc1ccc(-c2c(C)nn(COCC[Si](C)(C)C)c2C)cc1)C(C1CC1)C1CC1 ; Cc1nn(COCC[Si](C)(C)C)c(C)c1-c1ccc(NC(=O)C(C(=O)O)C(C2CC2)C2CC2)cc1]

A solution of KOH (235 mg, 4.19 mmol) in water (0.84 mL) was added to a solution of the compound of Preparation 8 (1.103 g, 2.098 mmol) in EtOH (2 mL) and the reaction mixture was stirred at room temperature for 18 hours. The reaction mixture was diluted with water (5 mL) and extracted with Et₂O (2×10 mL). The aqueous layer was acidified with HCl (5M aq. solution) to pH 1-2. The reaction mixture was then extracted with Et₂O (2×25 mL). The combined organic extracts were washed with brine solution (10 mL), dried over MgSO₄, filtered and concentrated in vacuo, to afford the title compound (866 mg, 83% yield). LCMS (Method 3) (ES): m/z 498.3 [M+H]⁺, RT=0.90 min.

### Preparation 10: tert-butyl N-[[2-(dicyclopropylmethyl)-3-[4-[3,5-dimethyl-1-(2-trimethylsilylethoxymethyl)pyrazol-4-yl]anilino]-3-oxo-propanoyl]amino]carbamate

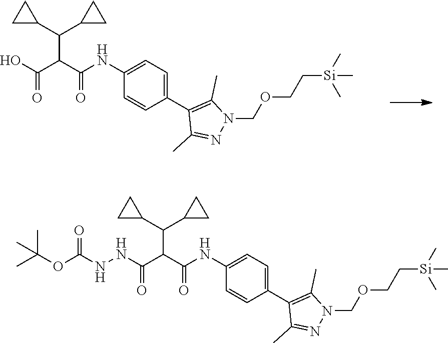

HATU (84.0 mg, 0.221 mmol) was added to a solution of the compound of Preparation 9 (100 mg, 0.201 mmol) and DIPEA (0.07 mL, 0.402 mmol) in DMF (3 mL) and stirred at room temperature for 30 minutes. Tert-butyl N-aminocarbamate (31.9 mg, 0.241 mmol) was added to the reaction mixture and stirred for 2 hours. The reaction mixture was purified directly by prep. acidic HPLC to afford the title compound (70 mg, 57% yield). 1H NMR (600 MHz, DMSO-d6) δ 9.90-9.56 (m, 2H), 8.98-8.27 (m, 1H), 7.72-7.52 (m, 2H), 7.30-7.14 (m, 2H), 5.34 (s, 2H), 3.59-3.48 (m, 2H), 3.31 (s, 1H), 2.25 (s, 3H), 2.13 (s, 3H), 1.47-1.21 (m, 9H), 1.17-1.01 (m, 1H), 0.93-0.62 (m, 4H), 0.47-0.12 (m, 8H), −0.04 (s, 9H); LCMS (Method 3) (ES): m/z 612.4 [M+H]⁺, RT=0.95 min.

### Preparation 11: 2-(dicyclopropylmethyl)-N-[4-[3,5-dimethyl-1-(2-trimethylsilylethoxymethyl)pyrazol-4-yl]phenyl]-3-hydrazino-3-oxo-propanamide hydrochloride

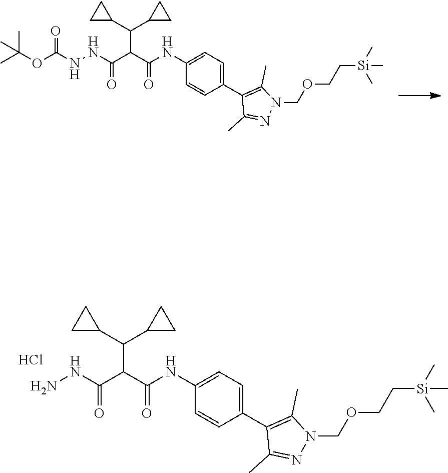 [SMILES: Cc1nn(COCC[Si](C)(C)C)c(C)c1-c1ccc(NC(=O)C(C(=O)NNC(=O)OC(C)(C)C)C(C2CC2)C2CC2)cc1 ; Cl ; Cc1nn(COCC[Si](C)(C)C)c(C)c1-c1ccc(NC(=O)C(C(=O)NN)C(C2CC2)C2CC2)cc1]

Hydrogen chloride (4M solution in 1,4-dioxane, 1 mL) was added to a solution of the compound of Preparation 10 (68 mg, 0.111 mmol) and the reaction mixture was stirred at room temperature for 90 minutes. The reaction mixture was diluted with MeOH (5 mL) then concentrated in vacuo to leave the title compound as a colourless solid (60 mg, 100% yield). LCMS (Method 3) (ES): m/z 512.3 [M+H]⁺, RT=0.85 min.

### Preparation 12: 3,3-dicyclopropyl-N-[4-[3,5-dimethyl-1-(2-trimethylsilylethoxymethyl)pyrazol-4-yl]phenyl]-2-(5-phenyl-4H-1,2,4-triazol-3-yl)propanamide

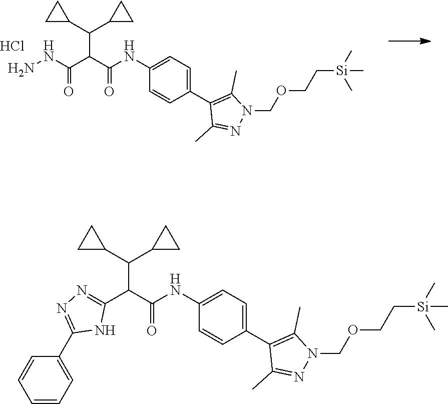 [SMILES: Cl ; Cc1nn(COCC[Si](C)(C)C)c(C)c1-c1ccc(NC(=O)C(C(=O)NN)C(C2CC2)C2CC2)cc1 ; Cc1nn(COCC[Si](C)(C)C)c(C)c1-c1ccc(NC(=O)C(c2nnc(-c3ccccc3)[nH]2)C(C2CC2)C2CC2)cc1]

DIPEA (0.20 mL, 1.10 mmol) was added to a solution of the compound of Preparation 11 (60.0 mg, 0.11 mmol) and ethyl benzenecarboximidate (16.0 mg, 0.11 mmol) in IPA (1 mL). The reaction mixture was stirred at 90° C. for 18 hours. The reaction mixture was diluted with MeOH (2 mL) and purified directly by prep. acidic HPLC to afford the title compound (7.0 mg, 11% yield). LCMS (Method 3) (ES): m/z 597.3 [M+H]⁺, RT=0.98 min.

### Preparation 13: 2-(dicyclopropylmethyl)-N-[4-[3,5-dimethyl-1-(2-trimethylsilylethoxymethyl)pyrazol-4-yl]phenyl]-N′-phenacyl-propanediamide

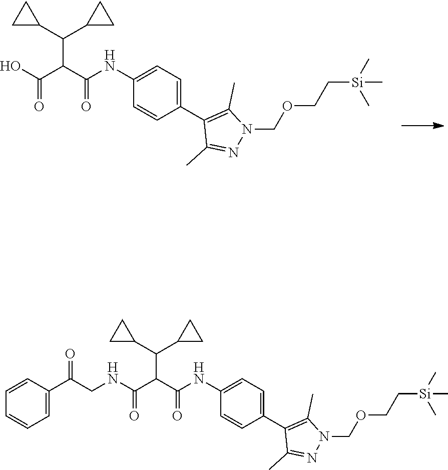 [SMILES: Cc1nn(COCC[Si](C)(C)C)c(C)c1-c1ccc(NC(=O)C(C(=O)O)C(C2CC2)C2CC2)cc1 ; Cc1nn(COCC[Si](C)(C)C)c(C)c1-c1ccc(NC(=O)C(C(=O)NCC(=O)c2ccccc2)C(C2CC2)C2CC2)cc1]

DIPEA (0.07 mL, 0.402 mmol) was added dropwise over 5 minutes to a solution of the compound of Preparation 9 (100 mg, 0.201 mmol), 2-amino-1-phenyl-ethanone hydrochloride (38.0 mg, 0.221 mmol) and HATU (84.0 mg, 0.221 mmol) in DMF (2 mL) and the reaction mixture was stirred at room temperature for 18 hours. The reaction mixture was purified directly by prep. acidic HPLC to afford the title compound (99.0 mg, 80% yield). LCMS (Method 3) (ES): m/z 615.3 [M+H]⁺, RT=0.99 min.

### Preparation 14: 3,3-dicyclopropyl-N-[4-[3,5-dimethyl-1-(2-trimethylsilylethoxymethyl)pyrazol-4-yl]phenyl]-2-(4-phenyl-1H-imidazol-2-yl)propanamide

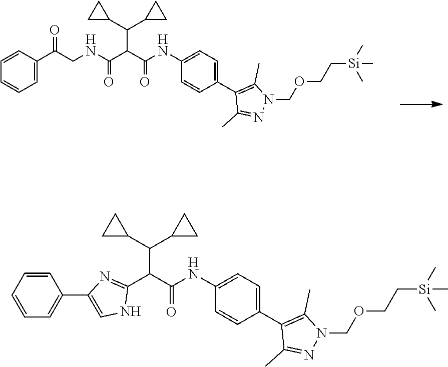 [SMILES: Cc1nn(COCC[Si](C)(C)C)c(C)c1-c1ccc(NC(=O)C(C(=O)NCC(=O)c2ccccc2)C(C2CC2)C2CC2)cc1 ; Cc1nn(COCC[Si](C)(C)C)c(C)c1-c1ccc(NC(=O)C(c2nc(-c3ccccc3)c[nH]2)C(C2CC2)C2CC2)cc1]

Ammonium acetate (62.1 mg, 0.805 mmol) was added to a solution of the compound of Preparation 13 (99 mg, 0.161 mmol) in toluene (2 mL) in a microwave vial. The vial was sealed and heated at 140° C. for 5 hours under microwave conditions. The cooled reaction mixture was concentrated in vacuo, re-dissolved in MeOH (2 mL) and filtered through a PTFE filter. The filtrate was purified by prep. acidic HPLC to afford the title compound (10 mg, 10% yield). LCMS (Method 3) (ES): m/z 595.3 [M+H]⁺, RT=0.93 min.

### Preparation 15: 2-(5-chloro-4-phenyl-1H-imidazol-2-yl)-3,3-dicyclopropyl-N-[4-[3,5-dimethyl-1-(2-trimethylsilylethoxymethyl)pyrazol-4-yl]phenyl]propanamide

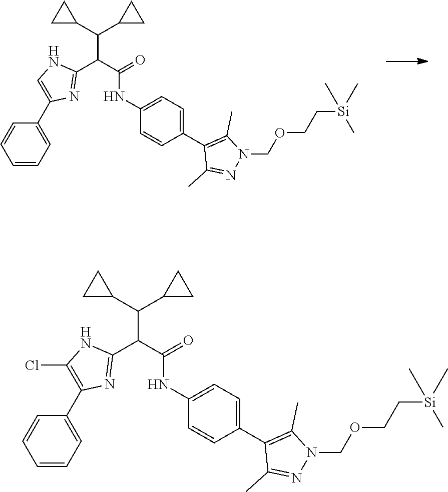 [SMILES: Cc1nn(COCC[Si](C)(C)C)c(C)c1-c1ccc(NC(=O)C(c2nc(-c3ccccc3)c[nH]2)C(C2CC2)C2CC2)cc1 ; Cc1nn(COCC[Si](C)(C)C)c(C)c1-c1ccc(NC(=O)C(c2nc(-c3ccccc3)c(Cl)[nH]2)C(C2CC2)C2CC2)cc1]

NCS (30.0 mg, 0.225 mmol) was added to a solution of the compound of Preparation 29 (10.0 mg, 0.017 mmol) in DMF (1 mL) and the reaction mixture was stirred at room temperature for 3 hours. The crude reaction mixture was purified directly by prep. basic HPLC to afford the title compound as a colourless solid (5.0 mg, 47% yield). ¹H NMR (600 MHz, CDCl₃) δ 7.73-7.68 (m, 4H), 7.47-7.41 (m, 2H), 7.35-7.30 (m, 1H), 7.24-7.19 (m, 2H), 5.37 (s, 2H), 4.24 (d, J=8.7 Hz, 1H), 3.68-3.51 (m, 2H), 2.30 (s, 3H), 2.23 (s, 3H), 1.00 (q, J=9.2 Hz, 1H), 0.94-0.88 (m, 2H), 0.87-0.77 (m, 1H), 0.70 (tt, J=8.4, 3.6 Hz, 1H), 0.48 (ddd, J=7.6, 4.3, 2.7 Hz, 2H), 0.39 (tt, J=8.8, 4.9 Hz, 1H), 0.28-0.11 (m, 4H), −0.02 (s, 9H), −0.13 (dq, J=10.0, 5.1 Hz, 1H); LCMS (Method 3) (ES): m/z 630.4 [M+H]⁺, RT=1.06 min.

### Preparation 16: 2-(dicyclopropylmethyl)-N′-[4-[3,5-dimethyl-1-(2-trimethylsilylethoxymethyl)pyrazol-4-yl]phenyl]propanediamide

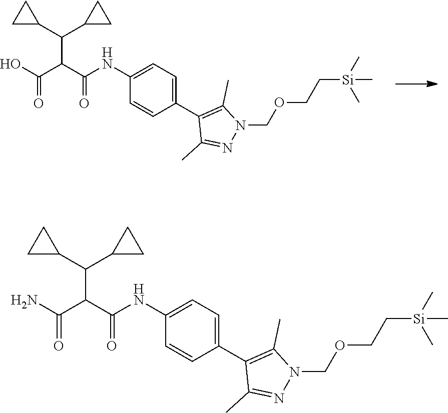 [SMILES: Cc1nn(COCC[Si](C)(C)C)c(C)c1-c1ccc(NC(=O)C(C(=O)O)C(C2CC2)C2CC2)cc1 ; Cc1nn(COCC[Si](C)(C)C)c(C)c1-c1ccc(NC(=O)C(C(N)=O)C(C2CC2)C2CC2)cc1]

DIPEA (0.182 mL, 1.045 mmol) was added to a solution of the compound of Preparation 9 (260 mg, 0.522 mmol), 1-hydroxybenztriazole hydrate (40.0 mg, 0.26 mmol), EDC (140 mg, 0.731 mmol) and ammonium chloride (55.9 mg, 1.045 mmol) in DMF (3 mL) and the reaction mixture was stirred at room temperature for 18 hours. Water (5 mL) was added to the reaction mixture and stirred for 10 minutes. The precipitate was collected by filtration, washed with water and dried under reduced pressure to afford the title compound (245 mg, 94% yield). 1H NMR (600 MHz, DMSO-d6) δ 9.97 (s, 1H), 7.68-7.53 (m, 2H), 7.36 (d, J=2.5 Hz, 1H), 7.24-7.13 (m, 3H), 5.34 (s, 2H), 3.58-3.53 (m, 2H), 2.25 (s, 3H), 2.13 (s, 3H), 1.00 (q, J=9.2 Hz, 1H), 0.88-0.80 (m, 2H), 0.80-0.67 (m, 2H), 0.46-0.14 (m, 8H), −0.04 (s, 9H); LCMS (Method 3) (ES): m/z 497.2 [M+H]⁺, RT=0.88 min.

### Preparation 17: 3,3-dicyclopropyl-N-[4-[3,5-dimethyl-1-(2-trimethylsilylethoxymethyl)pyrazol-4-yl]phenyl]-2-(4H-1,2,4-triazol-3-yl)propanamide

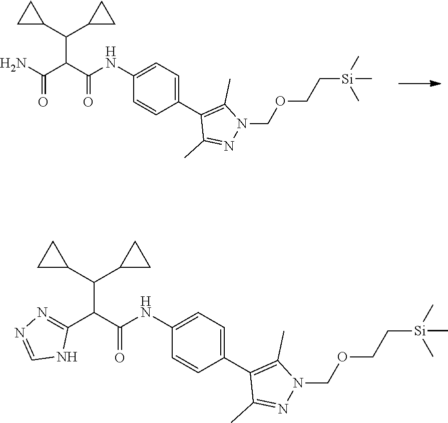 [SMILES: Cc1nn(COCC[Si](C)(C)C)c(C)c1-c1ccc(NC(=O)C(C(N)=O)C(C2CC2)C2CC2)cc1 ; Cc1nn(COCC[Si](C)(C)C)c(C)c1-c1ccc(NC(=O)C(c2nnc[nH]2)C(C2CC2)C2CC2)cc1]

1,1-dimethoxy-N,N-dimethyl-methanamine (0.102 mL, 0.725 mmol) was added to a suspension of the compound of Preparation 16 (240 mg, 0.483 mmol) in DCM (4.8 mL) and stirred at 50° C. for 1.5 hours. The reaction mixture was concentrated in vacuo, then re-dissolved in acetic acid (3 mL, 52.5 mmol). Hydrazine hydrate (0.30 mL, 4.80 mmol) was added and the resulting thick slurry was stirred at room temperature for 30 minutes. The reaction mixture was diluted with water (5 mL) and extracted with EtOAc (2×10 mL). The combined organic phase was concentrated in vacuo. The residue was dissolved in MeOH (4 mL), filtered through a PTFE filter and purified by prep. acidic HPLC to afford the title compound (192 mg, 76% yield). LCMS (Method 3) (ES): m/z 521.3 [M+H]⁺, RT=0.86 min.

### Preparation 18: 2-(5-bromo-4H-1,2,4-triazol-3-yl)-3,3-dicyclopropyl-N-[4-[3,5-dimethyl-1-(2-trimethylsilylethoxymethyl)pyrazol-4-yl]phenyl]propanamide

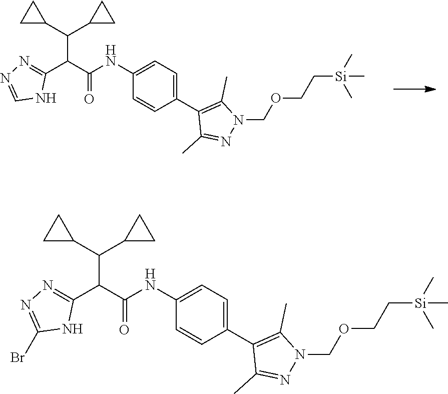 [SMILES: Cc1nn(COCC[Si](C)(C)C)c(C)c1-c1ccc(NC(=O)C(c2nnc[nH]2)C(C2CC2)C2CC2)cc1 ; Cc1nn(COCC[Si](C)(C)C)c(C)c1-c1ccc(NC(=O)C(c2nnc(Br)[nH]2)C(C2CC2)C2CC2)cc1]

A solution of benzyltrimethylammonium tribromide (82.4 mg, 0.211 mmol) in DCM (2 mL) was added to a vigorously stirring suspension of the compound of Preparation 17 (100 mg, 0.192 mmol) in DCM (4 mL) and NaOH (2M aq. solution, 0.29 mL, 0.58 mmol). The reaction mixture was stirred at room temperature for 18 hours. The reaction mixture was diluted with water (2 mL), the organic layer was collected and concentrated in vacuo. The residue was dissolved in MeOH (2 mL) and purified by prep. acidic HPLC to afford the title compound (96 mg, 83% yield). LCMS (Method 3) (ES): m/z 601.2 [M+H]⁺, RT=0.93 min.

### Preparation 19: 2-[5-bromo-4-(2-trimethylsilylethoxymethyl)-1,2,4-triazol-3-yl]-3,3-dicyclopropyl-N-[4-[3,5-dimethyl-1-(2-trimethylsilylethoxymethyl)pyrazol-4-yl]phenyl]propanamide (Mixture of Isomers)

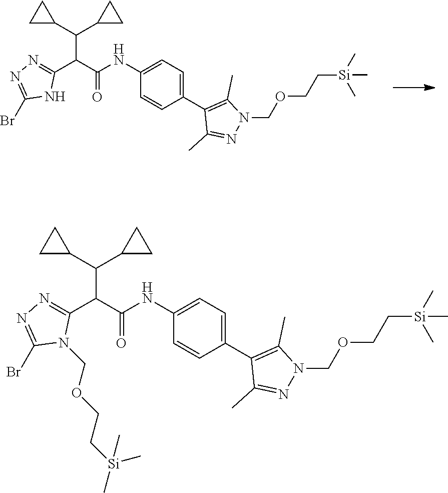 [SMILES: Cc1nn(COCC[Si](C)(C)C)c(C)c1-c1ccc(NC(=O)C(c2nnc(Br)[nH]2)C(C2CC2)C2CC2)cc1 ; Cc1nn(COCC[Si](C)(C)C)c(C)c1-c1ccc(NC(=O)C(c2nnc(Br)n2COCC[Si](C)(C)C)C(C2CC2)C2CC2)cc1]

SEM chloride (0.012 mL, 0.067 mmol) was added to a solution of the compound of Preparation 18 (20.0 mg, 0.033 mmol) and K₂CO₃ (23.0 mg, 0.167 mmol) in DMF (1 mL) and the reaction mixture was stirred at room temperature for 30 minutes. Water (0.3 mL) was added and the reaction mixture was transferred to a microwave vial for use in Preparation 20 (24 mg, assume 100% yield, mixture of SEM regioisomers). LCMS (Method 3) (ES): m/z 731.3 [M+H]⁺, RT=1.13 min.

### Preparation 20: 3,3-dicyclopropyl-N-[4-[3,5-dimethyl-1-(2-trimethylsilylethoxymethyl)pyrazol-4-yl]phenyl]-2-[5-(2-fluorophenyl)-4-(2-trimethylsilylethoxymethyl)-1,2,4-triazol-3-yl]propanamide (Mixture of Isomers)

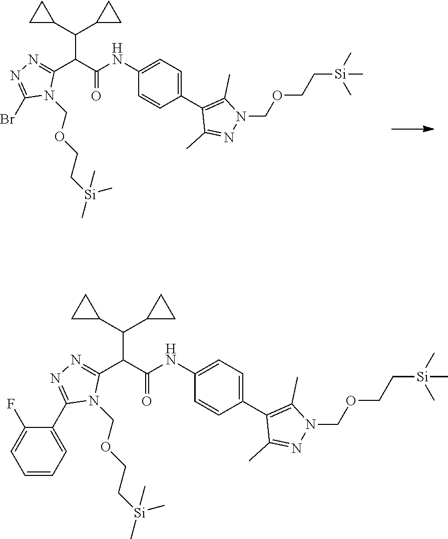 [SMILES: Cc1nn(COCC[Si](C)(C)C)c(C)c1-c1ccc(NC(=O)C(c2nnc(Br)n2COCC[Si](C)(C)C)C(C2CC2)C2CC2)cc1 ; Cc1nn(COCC[Si](C)(C)C)c(C)c1-c1ccc(NC(=O)C(c2nnc(-c3ccccc3F)n2COCC[Si](C)(C)C)C(C2CC2)C2CC2)cc1]

(2-Fluorophenyl)boronic acid (14.0 mg, 0.161 mmol) was added to the crude compound of Preparation 19 (24 mg, 0.033 mmol) and the reaction mixture was degassed with nitrogen for 10 minutes. Pd(dppf)Cl₂·DCM (2.7 mg, 0.003 mmol) was added and the reaction mixture was stirred at 90° C. for 30 minutes. The cooled reaction mixture was filtered through a PTFE filter and purified directly by prep. acidic HPLC to afford the title compound (12 mg, 48% yield) as a mixture of SEM regioisomers. LCMS (Method 3) (ES): m/z 745.5 [M+H]⁺, RT=1.20 min.

### Preparation 21: ethyl 2-(1-benzylimidazol-2-yl)acetate

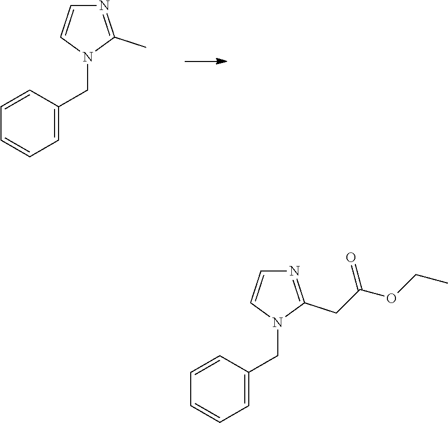 [SMILES: Cc1nccn1Cc1ccccc1 ; CCOC(=O)Cc1nccn1Cc1ccccc1]

Ethyl carbonochloridate (4.16 mL, 43.55 mmol) was added dropwise to a solution of I-benzyl-2-methyl-imidazole (5.00 g, 29.03 mmol) and triethylamine (8.09 mL, 58.06 mmol) in MeCN (100 mL) at 0° C. The reaction mixture was allowed to warm to room temperature and stirred for 18 hours. The reaction mixture was diluted with EtOAc (200 mL) and washed with water (50 mL), aqueous brine solution (50 mL), dried over MgSO₄, filtered and concentrated in vacuo. The obtained crude compound was purified by silica column chromatography (230-400 mesh), eluting with EtOAc (0-100%) in heptane, to afford the title compound as a yellow oil (2.05 g, 29% yield). 1H NMR (400 MHz, CDCl₃) δ 7.34 (qd, J=7.7, 6.8, 3.6 Hz, 3H), 7.12-7.07 (m, 2H), 7.02 (t, J=0.9 Hz, 1H), 6.87 (d, J=1.2 Hz, 1H), 5.13 (s, 2H), 4.12 (q, J=7.1 Hz, 2H), 3.74 (s, 2H), 1.26-1.19 (m, 3H); LCMS (Method 3) (ES): m/z 245.1 [M+H]⁺, RT=0.43 min.

### Preparation 22: ethyl (Z)-2-(1-benzylimidazol-2-yl)-3-cyclopropyl-prop-2-enoate

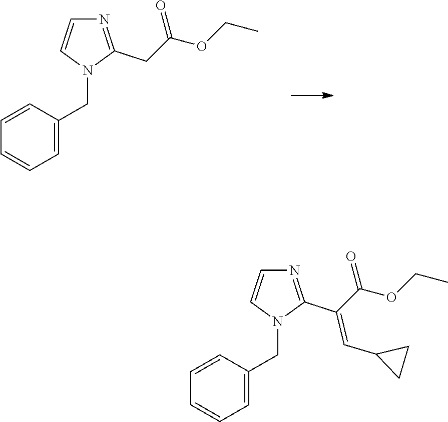 [SMILES: CCOC(=O)Cc1nccn1Cc1ccccc1 ; CCOC(=O)/C(=C\C1CC1)c1nccn1Cc1ccccc1]

According to the method of Preparation 1, the compound of Preparation 21 (500 mg, 2.05 mmol) was reacted to afford the title compound (275 mg, 48% yield). 1H NMR (400 MHz, DMSO-d6) δ 7.36-7.21 (m, 4H), 7.14-7.09 (m, 2H), 6.97 (t, J=0.9 Hz, 1H), 6.59 (d, J=11.0 Hz, 1H), 5.00 (s, 2H), 4.05 (q, J=7.1 Hz, 2H), 1.28-1.17 (m, 1H), 1.11 (t, J=7.1 Hz, 3H), 0.90-0.80 (m, 2H), 0.72-0.65 (m, 2H); LCMS (Method 3) (ES): m/z 298.1 [M+H]⁺, RT=0.54 min.

### Preparation 23: ethyl 2-(1-benzylimidazol-2-yl)-3,3-dicyclopropyl-propanoate

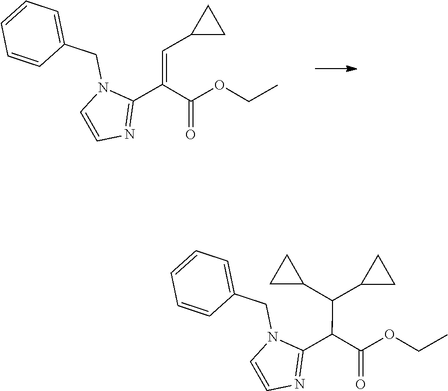 [SMILES: CCOC(=O)/C(=C\C1CC1)c1nccn1Cc1ccccc1 ; CCOC(=O)C(c1nccn1Cc1ccccc1)C(C1CC1)C1CC1]

According to the method of Preparation 2, the compound of Preparation 22 (270 mg, 0.911 mmol) was reacted to afford the title compound (182 mg, 59% yield). 1H NMR (400 MHz, DMSO-d6) δ 7.42-7.23 (m, 3H), 7.19 (s, 1H), 7.15-7.08 (m, 2H), 6.90 (s, 1H), 5.32 (d, J=16.1 Hz, 1H), 5.15 (d, J=16.1 Hz, 1H), 4.03-3.80 (m, 3H), 1.19-1.02 (m, 4H), 0.91-0.69 (m, 2H), 0.43-0.31 (m, 1H), 0.30-0.13 (m, 3H), 0.12-0.02 (m, 2H), −0.21 (s, 1H); LCMS (Method 3) (ES): m/z 340.2 [M+H]⁺, RT=0.63 min.

### Preparation 24: 2-(1-benzylimidazol-2-yl)-3,3-dicyclopropyl-propanoic acid

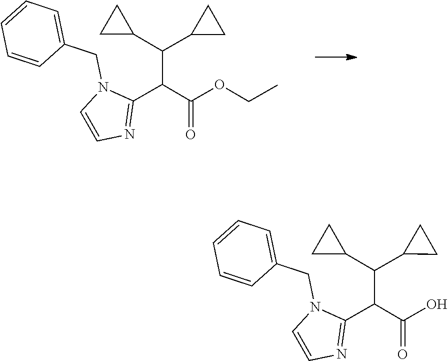 [SMILES: CCOC(=O)C(c1nccn1Cc1ccccc1)C(C1CC1)C1CC1 ; O=C(O)C(c1nccn1Cc1ccccc1)C(C1CC1)C1CC1]

A solution of KOH (59.6 mg, 1.06 mmol) in water (0.2 mL) was added to a solution of the compound of Preparation 23 (90.0 mg, 0.266 mmol) in EtOH (1 mL) and stirred at 40° C. for 18 hours. The reaction mixture was quenched with hydrogen chloride (5M aq. solution, 0.3 mL) and concentrated in vacuo to afford the crude title compound (82 mg, assume 100% yield). LCMS (Method 3) (ES): m/z 311.1 [M+H]⁺, RT=0.51 min.

### Preparation 25: 2-(1-benzylimidazol-2-yl)-3,3-dicyclopropyl-N-[4-[3,5-dimethyl-1-(2-trimethylsilylethoxymethyl)pyrazol-4-yl]phenyl]propanamide

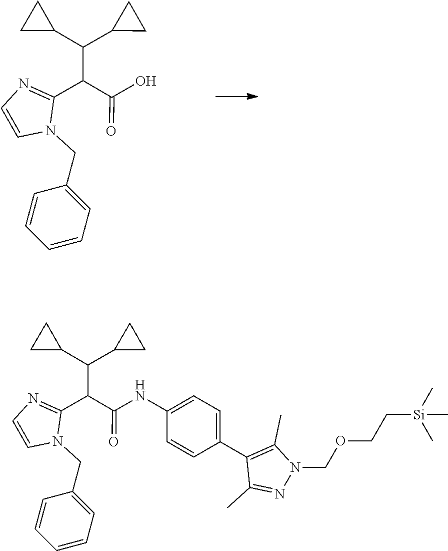 [SMILES: O=C(O)C(c1nccn1Cc1ccccc1)C(C1CC1)C1CC1 ; Cc1nn(COCC[Si](C)(C)C)c(C)c1-c1ccc(NC(=O)C(c2nccn2Cc2ccccc2)C(C2CC2)C2CC2)cc1]

According to the method of Preparation 8, the compound of Preparation 24 (81 mg, 0.26 mmol) was reacted with the compound of Preparation 7 (100 mg, 0.315 mmol) to afford the title compound after prep. acidic HPLC (96 mg, 61% yield). LCMS (Method 3) (ES): m/z 610.5 [M+H]⁺, RT=0.83 min.

### Preparation 26: 2-(1-benzylimidazol-2-yl)-3,3-dicyclopropyl-N-[4-(3,5-dimethyl-1H-pyrazol-4-yl)phenyl]propanamide

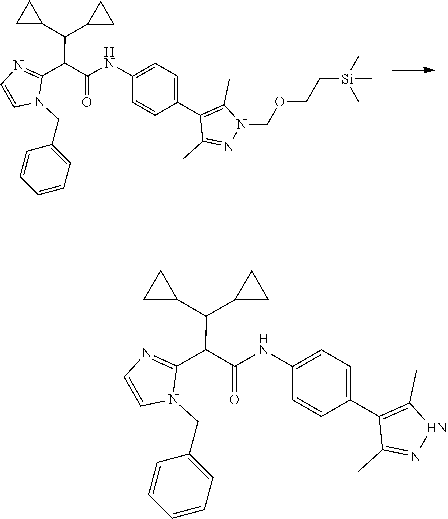 [SMILES: Cc1nn(COCC[Si](C)(C)C)c(C)c1-c1ccc(NC(=O)C(c2nccn2Cc2ccccc2)C(C2CC2)C2CC2)cc1 ; Cc1n[nH]c(C)c1-c1ccc(NC(=O)C(c2nccn2Cc2ccccc2)C(C2CC2)C2CC2)cc1]

TFA (1 mL) was added to a solution of the compound of Preparation 25 (96 mg, 0.157 mmol) in DCM (1 mL) and stirred at room temperature for 1 hour. The reaction mixture was concentrated in vacuo, re-dissolved in MeOH (2 mL) and purified directly by prep. acidic HPLC to afford the title compound (29 mg, 38% yield). 1H NMR (400 MHz, DMSO) δ 12.48 (s, 2H), 10.31 (s, 1H), 8.14 (s, 1H), 7.57-7.49 (m, 2H), 7.39-7.15 (m, 8H), 7.03 (s, 1H), 5.42-5.24 (m, 2H), 4.03 (d, J=8.7 Hz, 1H), 2.17 (s, 6H), 1.15 (q, J=9.0 Hz, 1H), 0.83-0.66 (m, 2H), 0.50-0.35 (m, 1H), 0.33-0.01 (m, 6H), −0.11-−0.24 (m, 1H); LCMS (Method 3) (ES): m/z 480.3 [M+H]⁺, RT=0.58 min.

### Preparation 27: O1-ethyl O3-(1-methyl-2-oxo-2-phenyl-ethyl) 2-(dicyclopropylmethyl)propanedioate

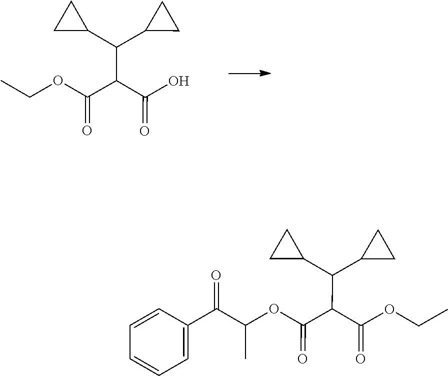 [SMILES: CCOC(=O)C(C(=O)O)C(C1CC1)C1CC1 ; CCOC(=O)C(C(=O)OC(C)C(=O)c1ccccc1)C(C1CC1)C1CC1]

Cs₂CO₃ (367 mg, 1.13 mmol) was added to a solution of the compound of Preparation 3 (170 mg, 0.751 mmol) in DMSO (20 mL) at room temperature. The reaction mixture was stirred for 10 minutes, 2-bromo-1-phenyl-propen-1-one (240 mg, 1.13 mmol) was added and the reaction mixture was stirred for a further 2 hours. The reaction mixture was partitioned between water (50 mL) and TBME (50 mL). The organic phase was collected. The aqueous phase was extracted with TBME (25 mL). The combined organic phase was dried over MgSO₄, filtered and concentrated in vacuo. The obtained crude compound was purified by silica column chromatography (230-400 mesh), eluting with EtOAc (25%) in heptane, to afford the title compound as a yellow oil (267 mg, 99% yield). LCMS (Method 4) (ES): m/z 358.4 [M+H]⁺, RT=0.93 min.

### Preparation 28: ethyl 3,3-dicyclopropyl-2-(5-methyl-4-phenyl-1H-imidazol-2-yl)propanoate

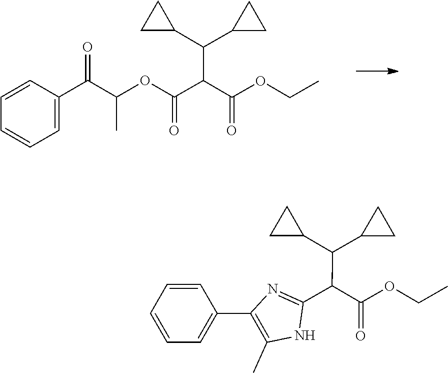 [SMILES: CCOC(=O)C(C(=O)OC(C)C(=O)c1ccccc1)C(C1CC1)C1CC1 ; CCOC(=O)C(c1nc(-c2ccccc2)c(C)[nH]1)C(C1CC1)C1CC1]

Ammonium acetate (347 mg, 4.51 mmol) and ammonium chloride (201 mg, 3.76 mmol) were added to a solution of the compound of Preparation 27 (267 mg, 0.745 mmol) in toluene (5 mL) and stirred in a sealed vial at 150° C. for 2 hours. The cooled reaction mixture was quenched with NaHCO₃ (saturated aq. solution, 20 mL) and extracted with TBME (2×20 mL). The combined organic phase was dried over MgSO₄, filtered and concentrated in vacuo. The obtained crude compound was purified by silica column chromatography (230-400 mesh), eluting with EtOAc (33%) in heptane, to afford an uncharacterised intermediate (88 mg). The intermediate was dissolved in AcOH (0.1 mL) and ammonium acetate (200 mg, 2.59 mmol) was added. The reaction mixture was stirred at room temperature for 1 hour. The reaction mixture was quenched with NaHCO₃ (saturated aq. solution, 2 mL) and extracted with TBME (2×10 mL). The combined organic phase was dried over MgSO₄, filtered and concentrated in vacuo. The obtained crude compound was purified by silica column chromatography (230-400 mesh), eluting with EtOAc (33%) in heptane, to afford the title compound as a colourless oil (55 mg, 21% yield). 1H NMR (400 MHz, CDCl₃) δ 9.75 (d, J=92.4 Hz, 1H), 7.64 (d, J=7.7 Hz, 1H), 7.40 (dd, J=11.5, 6.0 Hz, 3H), 7.24 (dt, J=14.5, 8.5 Hz, 1H), 4.22 (qq, J=7.1, 3.7 Hz, 2H), 4.16-4.04 (m, 1H), 2.41 (d, J=17.8 Hz, 3H), 1.31 (t, J=7.1 Hz, 3H), 0.76 (ttd, J=21.2, 8.5, 4.2 Hz, 3H), 0.55-0.42 (m, 2H), 0.42-0.25 (m, 2H), 0.18 (dq, J=9.8, 5.0 Hz, 2H), −0.01-−0.15 (m, 2H); LCMS (Method 3) (ES): m/z 339.3 [M+H]⁺, RT=0.63 min.

### Preparation 29: 3,3-dicyclopropyl-2-(5-methyl-4-phenyl-1H-imidazol-2-yl)propanoic acid

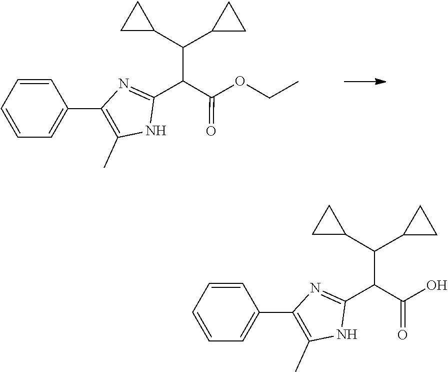 [SMILES: CCOC(=O)C(c1nc(-c2ccccc2)c(C)[nH]1)C(C1CC1)C1CC1 ; Cc1[nH]c(C(C(=O)O)C(C2CC2)C2CC2)nc1-c1ccccc1]

NaOH (65 mg, 1.63 mmol) was added to a solution of the compound of Preparation 28 (50 mg, 0.145 mmol) in MeOH (4 mL) and water (1 mL). The reaction mixture was stirred at 50° C. for 1 hour. The cooled reaction mixture was purified directly by prep. acidic HPLC to afford the title compound as a colourless solid (29 mg, 63%).

### Preparation 30: 3,3-dicyclopropyl-N-[4-[3,5-dimethyl-1-(2-trimethylsilylethoxymethyl)pyrazol-4-yl]phenyl]-2-(5-methyl-4-phenyl-1H-imidazol-2-yl)propanamide

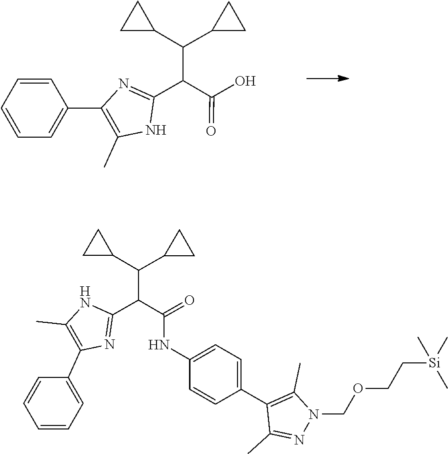 [SMILES: Cc1[nH]c(C(C(=O)O)C(C2CC2)C2CC2)nc1-c1ccccc1 ; Cc1nn(COCC[Si](C)(C)C)c(C)c1-c1ccc(NC(=O)C(c2nc(-c3ccccc3)c(C)[nH]2)C(C2CC2)C2CC2)cc1]

According to the method of Preparation 8, the compound of Preparation 29 (27 mg, 0.087 mmol) was reacted with the compound of Preparation 7 (35 mg, 0.110 mmol) to afford the title compound after prep. basic HPLC (25 mg, 48% yield). LCMS (Method 4) (ES): m/z 610.3 [M+H]⁺, RT=0.98 min.

### Preparation 31: 4-fluoro-1-methyl-pyrazole

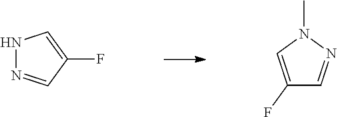

Methyl iodide (1.08 mL, 17.4 mmol) was added to a solution of 4-fluoro-1H-pyrazole (1.25 g, 14.5 mmol) and cesium carbonate (4.73 g, 14.5 mmol) in DMF (10 mL) and stirred at room temperature in a closed vial for 2 hours. The reaction mixture was diluted with Et₂O (40 mL) and water (20 mL) and separated. The aqueous phase was then extracted with Et₂O (20 mL), and the combined organic phases were washed with water (20 mL), washed with brine (20 mL), dried over MgSO₄, filtered and and concentrated under slightly reduced pressure (the title compound is volatile) to give the title compound (1.05 g, 58% yield) as a colourless oil. 1H NMR (400 MHz, CDCl₃) δ 7.30 (d, J=4.2 Hz, 1H), 7.24 (d, J=4.8 Hz, 1H), 3.82 (s, 3H).

### Preparation 32: 4-fluoro-2-methyl-pyrazole-3-carboxylic acid

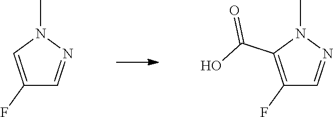

n-BuLi (4.40 mL, 10.9 mmol) was added to a solution of the compound of Preparation 31 (1.04 g, 8.42 mmol) in dry ether (25 mL) and cooled to −10° C. under a nitrogen atmosphere. A suspension formed and the reaction mixture was warmed to room temperature and stirred for 30 minutes, before CO₂ (g) was bubbled through the reaction mixture for 20 minutes. Water (40 mL) was added to the reaction mixture and the two phases were separated. The water phase was washed with Et₂O (2×20 mL), mixed with EtOAc (40 mL), acidified to pH 3-4 using NaHSO₄ solution (1M, 13 mL) and separated. The aqueous phase was extracted with EtOAc (2×20 mL) and the combined organic phases were washed with brine (20 mL), dried over MgSO₄, filtered and concentrated under reduced pressure to give the title compound (862 mg, 71% yield) as a yellow solid. 1H NMR (400 MHz, DMSO-d6) δ 13.62 (s, 1H), 7.60 (d, J=4.3 Hz, 1H), 4.01 (d, J=1.0 Hz, 3H); LCMS (Method 3) (ES): m/z 143.0 [M−H]⁻, RT=0.26 min.

### Preparation 33: tert-butyl-dimethyl-[3-[5-(4,4,5,5-tetramethyl-1,3,2-dioxaborolan-2-yl)pyrazol-1-yl]propoxy]silane

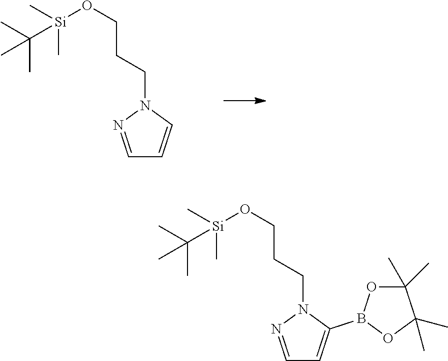 [SMILES: CC(C)(C)[Si](C)(C)OCCCn1cccn1 ; CC1(C)OB(c2ccnn2CCCO[Si](C)(C)C(C)(C)C)OC1(C)C]

n-BuLi (2.5M in hexanes, 1.8 mL, 4.5 mmol) was added dropwise to a solution of tert-butyl-dimethyl-(3-pyrazol-1-ylpropoxy)silane (0.78 g, 3.2 mmol) in dry THF (10 mL) at −75° C. under a nitrogen atmosphere and stirred for 30 minutes. 2-Isopropoxy-4,4,5,5-tetramethyl-1,3,2-dioxaborolane (0.73 mL, 3.2 mmol) was added and the reaction mixture was allowed to warm to room temperature. After 1 hour the mixture was quenched with NH₄Cl (aqueous solution, 5 mL) and extracted with in Et₂O (25 mL), dried over MgSO₄, filtered and concentrated under reduced pressure. The material was dissolved in MeCN and purified by prep. acidic HPLC to afford the title compound (199 mg, 17% yield). 1H NMR (400 MHz, CDCl₃) δ 7.49 (d, J=1.9 Hz, 1H), 6.70 (d, J=1.9 Hz, 1H), 4.52-4.43 (m, 2H), 3.66 (t, J=6.4 Hz, 2H), 2.09-2.03 (m, 2H), 1.33 (s, 12H), 0.89 (s, 9H), 0.04 (s, 6H); LCMS (Method 3) (ES): m/z 367.4 [M+H]⁺, RT=1.08 min.

### Preparation 34: 3,3-dicyclopropyl-N-[4-[3,5-dimethyl-1-(2-trimethylsilylethoxymethyl)pyrazol-4-yl]phenyl]-2-(4-fluoro-5-phenyl-1H-imidazol-2-yl)propanamide

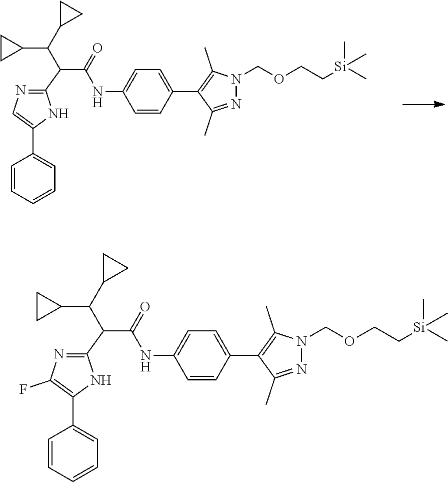 [SMILES: Cc1nn(COCC[Si](C)(C)C)c(C)c1-c1ccc(NC(=O)C(c2ncc(-c3ccccc3)[nH]2)C(C2CC2)C2CC2)cc1 ; Cc1nn(COCC[Si](C)(C)C)c(C)c1-c1ccc(NC(=O)C(c2nc(F)c(-c3ccccc3)[nH]2)C(C2CC2)C2CC2)cc1]

To a solution of the compound of Preparation 14 (10 mg, 0.017 mmol) in DMF (1 mL) and water (0.1 mL) was added Selectfluor (10 mg, 0.028 mmol) at room temperature. The reaction mixture was stirred for 10 minutes before it was quenched with sat. aq. NaHCO₃ solution and extracted with TBME. The organic phase was concentrated under reduced pressure, dissolved in MeCN and purified by prep. acidic HPLC to give the title compound (2 mg, 19% yield). 1H NMR (600 MHz, CDCl₃) δ 10.64 (s, 1H), 9.56 (s, 1H), 7.67 (d, J=8.5 Hz, 2H), 7.55 (d, J=7.8 Hz, 2H), 7.41 (t, J=7.8 Hz, 2H), 7.25-7.23 (m, 3H), 5.38 (s, 2H), 4.00 (d, J=8.0 Hz, 1H), 3.65-3.60 (m, 2H), 2.30 (s, 3H), 2.24 (s, 3H), 0.94-0.86 (m, 3H), 0.82-0.73 (m, 2H), 0.52-0.11 (m, 8H), −0.015 (s, 9H); LCMS (Method 3) (ES): m/z 612.5 [M−H]⁻, RT=1.04 min.

### Preparation 35: Benzyl protected ethyl 2-(5-bromo-1H-imidazol-2-yl)-3,3-dicyclopropyl-propanoate

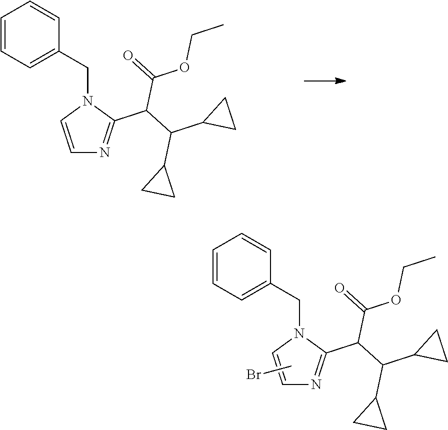

To a solution of the compound of Preparation 23 (90 mg, 0.27 mmol) in DCM (2 mL) was added NBS (47 mg, 0.27 mmol) at room temperature and stirred for 1 hour. The reaction mixture was diluted with DCM (5 mL), washed with water (2×2 mL), dried over MgSO₄, filtered and concentrated under reduced pressure to give the crude title compound (98 mg, 88% yield). LCMS (Method 3) (ES): m/z 417.2 [M+H]⁺, RT=0.91 min.

### Preparation 36: Benzyl protected ethyl 3,3-dicyclopropyl-2-[5-(2-methoxyphenyl)-1H-imidazol-2-yl]propanoate

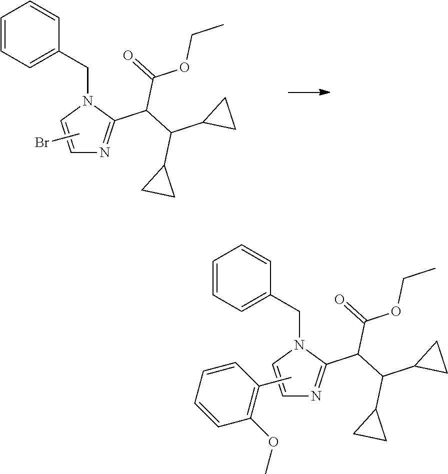

Aqueous potassium carbonate (1 mg/mL, 0.24 mL, 0.35 mmol) was added to a solution of the compound of Preparation 35 (49 mg, 0.12 mmol) and (2-methoxyphenyl)boronic acid (36 mg, 0.23 mmol) in DMF (1 mL) and degassed with nitrogen for 5 minutes. Pd(dppf)Cl₂·DCM (10 mg, 0.012 mmol) was added and the reaction mixture was stirred at 90° C. for 20 minutes. The cooled reaction mixture was filtered through a PTFE filter and purified directly by prep. acidic HPLC to afford the title compound (30 mg, 57% yield). LCMS (Method 3) (ES): m/z 446.3 [M+H]⁺, RT=0.78 min.

### Preparation 37: Benzyl protected 3,3-dicyclopropyl-2-[5-(2-methoxyphenyl)-1H-imidazol-2-yl]propanoic acid

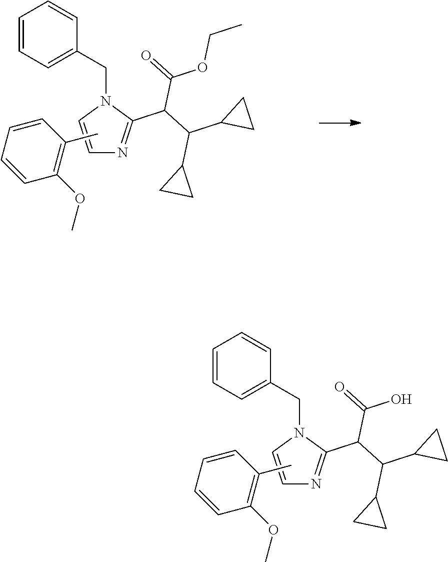

KOH (34 mg, 0.61 mmol) in water (1 mL) was added to a solution of the compound of preparation 36 (27 mg, 0.061 mmol) in EtOH (2 mL) and stirred at room temperature for 18 hours and at 2 hours at 60° C. Hydrogen chloride solution (5M, 0.5 mL) was added and the reaction mixture was concentrated under reduced pressure to give the crude title compound (25 mg, assume 100% yield). LCMS (Method 3) (ES): m/z 417.3 [M+H]⁺, RT=0.65 min.

### Preparation 38: Benzyl protected 3,3-dicyclopropyl-N-[4-[3,5-dimethyl-1-(2-trimethylsilylethoxymethyl)pyrazol-4-yl]phenyl]-2-[5-(2-methoxyphenyl)-1H-imidazol-2-yl]propanamide

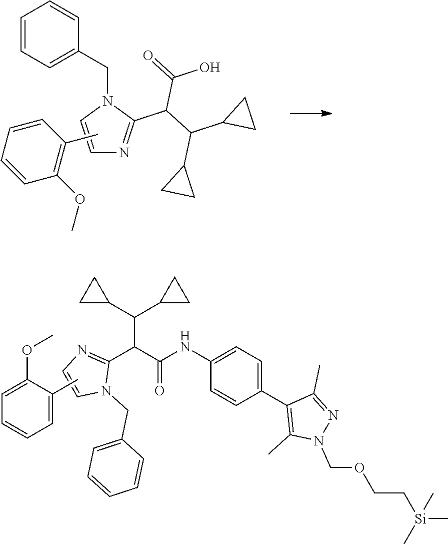

According to the method of Preparation 8, the compound of Preparation 37 (25 mg, 0.061 mmol) was reacted with the compound of Preparation 7 (21 mg, 0.067 mmol) to afford the title compound after prep. acidic HPLC (18 mg, 41% yield). LCMS (Method 3) (ES): m/z 718.4 [M+H]⁺, RT=1.00 min.

### Preparation 39: Benzyl protected 3,3-dicyclopropyl-N-[4-(3,5-dimethyl-1H-pyrazol-4-yl)phenyl]-2-[5-(2-methoxyphenyl)-1H-imidazol-2-yl]propanamide

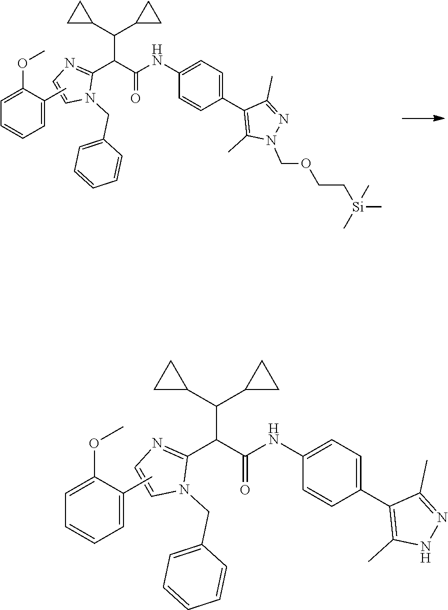

According to the method of Example 1 the compound of Preparation 38 (10 mg, 0.022 mmol) was reacted to afford the crude title compound (14 mg, 100% yield). LCMS (Method 3) (ES): m/z 586.4 [M+H]⁺, RT=0.73 min.

### Preparation 40: ethyl 2-[(tert-butoxycarbonylamino)carbamoyl]-3,3-dicyclopropyl-propanoate

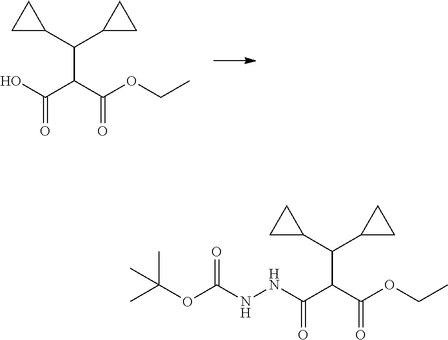 [SMILES: CCOC(=O)C(C(=O)O)C(C1CC1)C1CC1 ; CCOC(=O)C(C(=O)NNC(=O)OC(C)(C)C)C(C1CC1)C1CC1]

According to the method of Preparation 10 the compound of Preparation 3 (408 mg, 1.80 mmol) was reacted for 5 days. The reaction mixture was diluted with Et₂O (10 mL) and washed successively with saturated sodium hydrogen carbonate solution (in water, 3 mL), sodium hydrogen sulphate solution (10% in water, 3 mL) and saturated brine. The organic phase was dried over MgSO₄, filtered and concentrated under reduced pressure to afford the crude title compound (524 mg, 85% yield). 1H NMR (400 MHz, DMSO-d6) δ 9.76 (s, 1H), 8.82 (s, 1H), 4.14-3.99 (m, 2H), 3.41-3.36 (m, 1H), 1.38 (S, 9H), 1.18 (t,J=7.1 Hz, 3H), 1.01-0.59 (m, 3H), 0.45-0.07 (m, 9H).

### Preparation 41: ethyl 2-(dicyclopropylmethyl)-3-hydrazino-3-oxo-propanoate hydrochloride

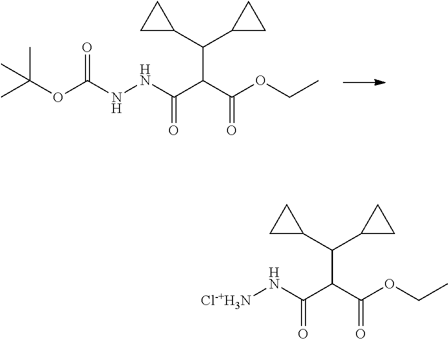

Hydrogen chloride (4M solution in 1,4-dioxane, 2 mL) was added to a solution of the compound of Preparation 40 (149 mg, 0.438 mmol) in EtOH (0.5 mL) and the reaction mixture was stirred at room temperature for 1 hour. The reaction mixture was concentrated in vacuo to leave the crude title compound (121 mg, assume 100% yield). 1H NMR (400 MHz, DMSO-d6) δ 11.27 (s, 1H), 4.14-4.06 (m, 2H), 3.70-3.66 (m, 1H), 1.19 (t,J=7.1 Hz, 3H), 0.93 (q, J=9.0 Hz, 1H), 0.85-0.71 (m, 2H), 0.49-0.03 (m, 8H).

### Preparation 42: ethyl 2-(5-cyclopentyl-4H-1,2,4-triazol-3-yl)-3,3-dicyclopropyl-propanoate

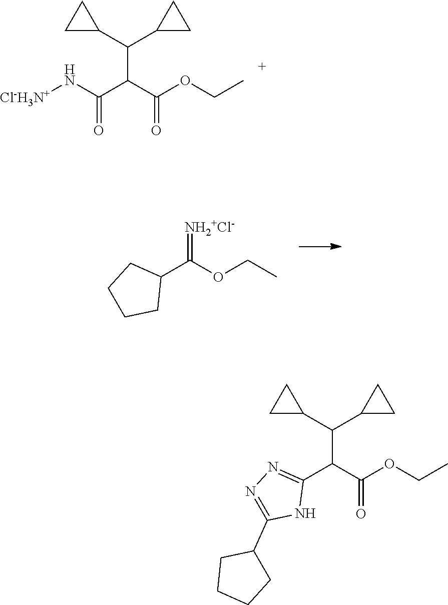

A mixture of the compound of Preparation 41 (120 mg, 0.43 mmol), ethyl cyclopentanecarbimidate hydrochloride (77 mg, 0.43 mmol) and DIPEA (1.0 mL, 5.7 mmol) in IPA (3 mL) was heated to 100° C. for 18 hours. The mixture was then concentrated under reduced pressure, dissolved in DCM and purified by silica column chromatography (230-400 mesh), eluting with EtOAc (0-100%) in heptane, to afford the title compound (45 mg, 33% yield). 1H NMR (400 MHz, CDCl₃) δ 4.20 (qd, J=7.2, 2.8 Hz, 2H), 4.14 (d, J=6.9 Hz, 1H), 3.18 (quintet, J=8.0 Hz, 1H), 2.12-1.97 (m, 2H), 1.89-1.56 (m, 6H), 1.28 (t, J=7.1 Hz, 3H), 0.90-0.63 (m, 3H), 0.51-0.29 (m, 3H), 0.26-0.10 (m, 3H), −0.03-−0.15 (m, 2H); LCMS (Method 3) (ES): m/z 336.3 [M+H]⁺, RT=0.56 min.

### Preparation 43: 2-(5-cyclopentyl-4H-1,2,4-triazol-3-yl)-3,3-dicyclopropyl-propanoic acid

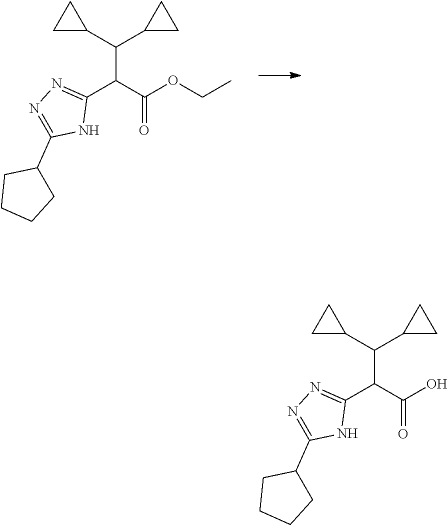 [SMILES: CCOC(=O)C(c1nnc(C2CCCC2)[nH]1)C(C1CC1)C1CC1 ; O=C(O)C(c1nnc(C2CCCC2)[nH]1)C(C1CC1)C1CC1]

KOH (33 mg, 0.58 mmol) in water (0.058 mL) was added to a solution of the product of Preparation 42 (46 mg, 0.14 mmol) in EtOH (0.58 mL) and stirred for 18 hours at room temperature. HCl (5M, 1 mL) was added and the reaction mixture was concentrated in vacuo. Water (2 mL) and EtOAc (2 mL) were added and the reaction mixture was concentrated in vacuo to leave the crude title compound (42 mg, assume 100% yield). LCMS (Method 3) (ES): m/z 288.2 [M−H]⁻, RT=0.60 min.

### Preparation 44: 2-(5-cyclopentyl-4H-1,2,4-triazol-3-yl)-3,3-dicyclopropyl-N-[4-[3,5-dimethyl-1-(2-trimethylsilylethoxymethyl)pyrazol-4-yl]phenyl]propanamide

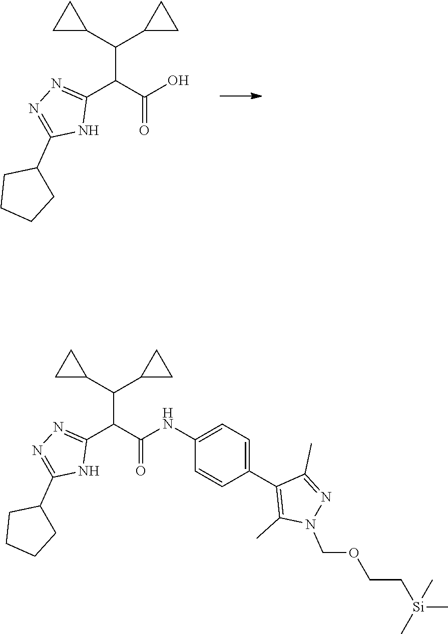 [SMILES: O=C(O)C(c1nnc(C2CCCC2)[nH]1)C(C1CC1)C1CC1 ; Cc1nn(COCC[Si](C)(C)C)c(C)c1-c1ccc(NC(=O)C(c2nnc(C3CCCC3)[nH]2)C(C2CC2)C2CC2)cc1]

According to the method of Preparation 8, the compound of Preparation 43 (42 mg, 0.14 mmol) was reacted with the compound of Preparation 7 (55 mg, 0.17 mmol) to afford the title compound after prep. acidic HPLC (24 mg, 28% yield). LCMS (Method 3) (ES): m/z 589.4 [M+H]⁺, RT=0.99 min.

### Preparation 45: ethyl 2-carbamoyl-3,3-dicyclopropyl-propanoate

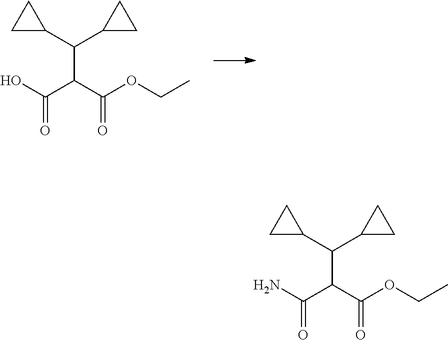 [SMILES: CCOC(=O)C(C(=O)O)C(C1CC1)C1CC1 ; CCOC(=O)C(C(N)=O)C(C1CC1)C1CC1]

According to the method of Preparation 16, the compound of Preparation 3 (1.2 g, 5.3 mmol) was reacted for 18 hours. The reaction mixture was diluted with Et₂O (50 mL) and washed with water (25 mL), the water phase was then extracted with Et₂O (25 mL). The combined organic phases were washed successively with a solution of saturated aq. sodium hydrogen carbonate (20 mL), sodium hydrogen sulphate solution (10% in water, 20 mL) and saturated brine, then dried over MgSO₄, filtered and concentrated under reduced pressure to afford the crude title compound (0.51 g, 42% yield). 1H NMR (400 MHz, CDCl₃) δ 7.03 (s, 1H), 5.54 (s, 1H), 4.21 (q, J=7.2 Hz, 2H), 3.40 (d, J=7.0 Hz, 1H), 1.30 (t, J=7.2 Hz, 3H), 0.91-0.72 (m, 3H), 0.58-0.13 (m, 8H).

### Preparation 46: ethyl 3,3-dicyclopropyl-2-(4H-1,2,4-triazol-3-yl)propanoate

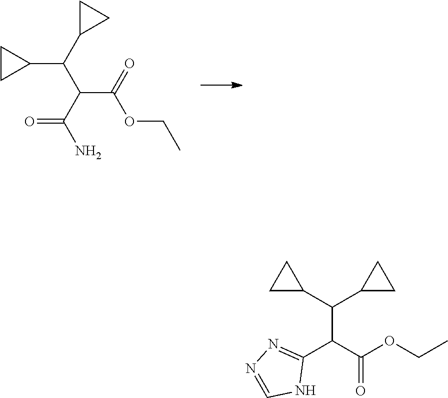 [SMILES: CCOC(=O)C(C(N)=O)C(C1CC1)C1CC1 ; CCOC(=O)C(c1nnc[nH]1)C(C1CC1)C1CC1]

According to the method of Preparation 17, the compound of Preparation 45 (1.2 g, 5.3 mmol) was reacted for 2 hours before the reaction mixture was diluted with water (25 mL) and extracted with EtOAc (2×50 mL). The combined organic phases were washed with water (20 mL), washed with brine (20 mL), dried over MgSO₄, filtered and concentrated under reduced pressure to afford the crude title compound (0.74 g, assume 100% yield) as a pale yellow oil. LCMS (Method 3) (ES): m/z 250.2 [M+H]⁺, RT=0.60 min.

### Preparation 47: ethyl 2-(5-bromo-4H-1,2,4-triazol-3-yl)-3,3-dicyclopropyl-propanoate

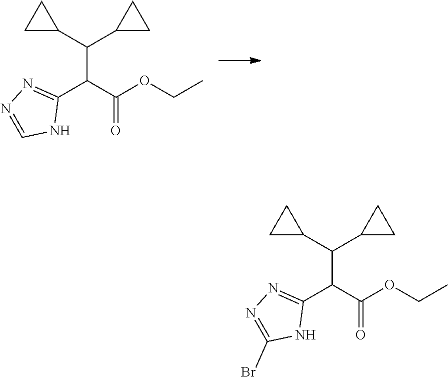 [SMILES: CCOC(=O)C(c1nnc[nH]1)C(C1CC1)C1CC1 ; CCOC(=O)C(c1nnc(Br)[nH]1)C(C1CC1)C1CC1]

NBS (625 mg, 3.51 mmol) was added to a solution of the crude product of Preparation 46 (730 mg, 2.93 mmol) in DCM (15 mL) and stirred at room temperature for 2 hours before the mixture was diluted with DCM (30 mL), washed with water (2×15 mL) dried over MgSO₄, filtered and concentrated under reduced pressure. The concentrate was dissolved in DCM and purified by silica column chromatography (230-400 mesh), eluting with EtOAc (0-100%) in heptane. The product still contained starting material and was dissolved in DCM (10 mL). NBS (1.17 g, 6.58 mmol) was added in portions and the reaction was heated for 18 hours at 60° C. and 1 hour at 100° C. The mixture was diluted with DCM (20 mL), washed with a solution of saturated aq. sodium hydrogen carbonate (10 mL), dried over MgSO₄, filtered and concentrated under reduced pressure. The residue was dissolved in DCM and purified by silica column chromatography (230-400 mesh), eluting with EtOAc (0-100%) in heptane giving the title compound (132 mg, 14% yield). 1H NMR (400 MHz, CDCl₃) δ 4.26 (q, J=7.2 Hz, 2H), 4.21 (d, J=4.9 Hz, 1H), 1.32 (t, J=7.1 Hz, 3H), 0.91-0.65 (m, 3H), 0.55-0.47 (m, 2H), 0.44-0.35 (m, 2H), 0.23-0.15 (m, 2H), 0.047-−0.032 (m, 1H), −0.098-−0.17 (m, 1H).

### Preparation 48: SEM Protected Ethyl 2-(5-bromo-4H-1,2,4-triazol-3-yl)-3,3-dicyclopropyl-propanoate (Mixture of Isomers)

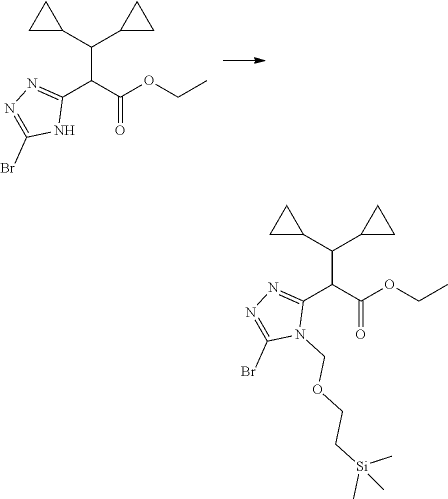 [SMILES: CCOC(=O)C(c1nnc(Br)[nH]1)C(C1CC1)C1CC1 ; CCOC(=O)C(c1nnc(Br)n1COCC[Si](C)(C)C)C(C1CC1)C1CC1]

SEM chloride (0.12 mL, 0.68 mmol) was added to a solution of the compound of Preparation 47 (131 mg, 0.399 mmol) and potassium carbonate (221 mg, 1.60 mmol) in DMF (1 mL) and the reaction mixture was stirred at room temperature for 1 hour. The mixture was diluted with Et₂O (20 mL) and water (10 mL) and separated. The aqueous phase was extracted with Et₂O (10 mL) and the combined organic phases washed with water (5 mL), washed with brine (5 mL), dried over MgSO₄, filtered and concentrated under reduced pressure giving the crude title compound as a mixture of SEM regioisomers (183 mg, assume 100% yield).

### Preparation 49: SEM Protected Ethyl 3,3-dicyclopropyl-2-[5-(2-isopropylpyrazol-3-yl)-4H-1,2,4-triazol-3-yl]propanoate (Mixture of Isomers)

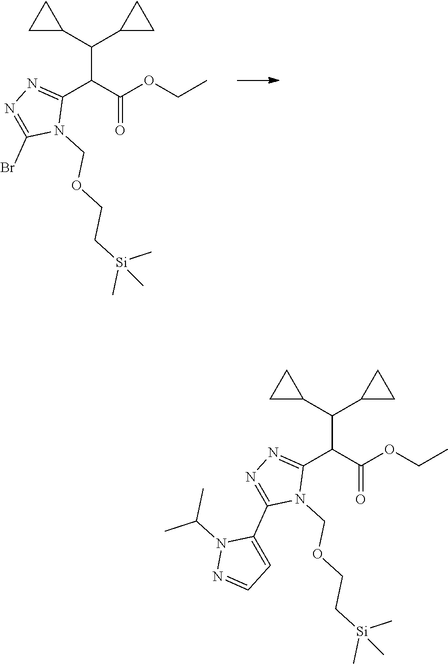 [SMILES: CCOC(=O)C(c1nnc(Br)n1COCC[Si](C)(C)C)C(C1CC1)C1CC1 ; CCOC(=O)C(c1nnc(-c2ccnn2C(C)C)n1COCC[Si](C)(C)C)C(C1CC1)C1CC1]

According to the method of Preparation 36, the compound of Preparation 48 (183 mg, 0.399 mmol) and 1-isopropyl-5-(4,4,5,5-tetramethyl-1,3,2-dioxaborolan-2-yl)pyrazole (189 mg, 0.798 mmol) were reacted to afford the title compound after prep. acidic HPLC (85 mg, 45% yield) as a mixture of SEM regioisomers. LCMS (Method 3) (ES): m/z 488.5 [M+H]⁺, RT=1.07 and 1.08 min.

### Preparation 50: 2-[[3,5-dimethyl-4-(4,4,5,5-tetramethyl-1,3,2-dioxaborolan-2-yl)pyrazol-1-yl]methoxy]ethyl-trimethyl-silane

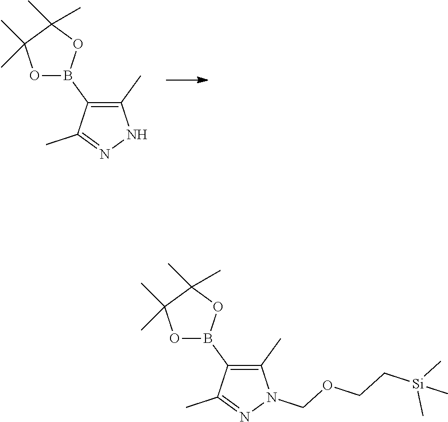

SEM chloride (5.78 mL, 32.6 mmol) was added to a solution of 3,5-dimethyl-4-(4,4,5,5-tetramethyl-1,3,2-dioxaborolan-2-yl)-1H-pyrazole (5.00 g, 22.5 mmol) and K₂CO₃ (6.22 g, 45.0 mmol) in NMP (34 mL) and stirred at room temperature for 18 hours. The reaction mixture was diluted with EtOAc (150 mL) and filtered to remove precipitate. The filtrate was successively washed with water (2×50 mL), saturated aq. NaHCO₃ (50 mL) and brine solution (50 mL), dried over MgSO₄, filtered and concentrated in vacuo. The obtained crude compound was purified by silica column chromatography (230-400 mesh), eluting with EtOAc (0-30%) in heptane, to afford the title compound as a colourless oil (5.85 g, 74% yield). 1H NMR (300 MHz, DMSO-d6) δ 5.27 (s, 2H), 3.60-3.41 (m, 2H), 2.36 (s, 3H), 2.17 (s, 3H), 1.25 (s, 12H), 0.97-0.71 (m, 2H), −0.05 (s, 9H).

### Preparation 51: 5-[3,5-dimethyl-1-(2-trimethylsilylethoxymethyl)pyrazol-4-yl]-6-fluoro-pyridin-2-amine

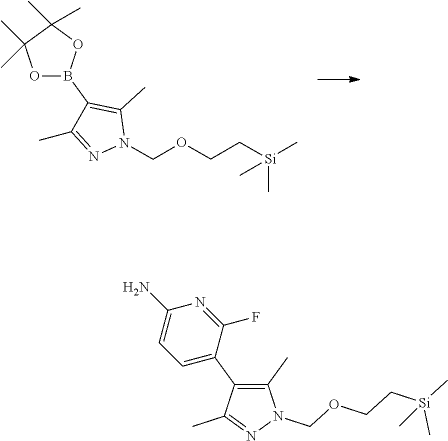

According to the method of Preparation 36 the compound of Preparation 50 (1.50 g, 4.26 mmol) was reacted with 5-bromo-6-fluoro-pyridin-2-amine (0.78 g, 4.10 mmol) to afford the title compound as a pale yellow solid (1.36 g, 99% yield). LCMS (METHOD 3) (ES): m/z 337.2 [M+H]⁺, RT=0.80 min.

### Preparation 52: SEM Protected 3,3-dicyclopropyl-N-[5-[3,5-dimethyl-1-(2-trimethylsilylethoxymethyl)pyrazol-4-yl]-6-fluoro-2-pyridyl]-2-[5-(2-isopropylpyrazol-3-yl)-4H-1,2,4-triazol-3-yl]propanamide (Mixture of Isomers)

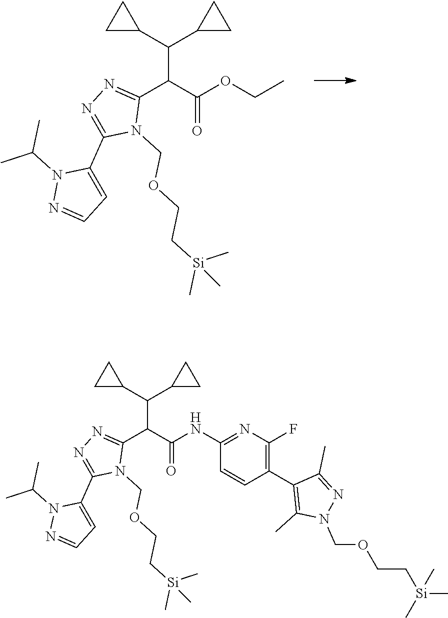

Tert-butylmagnesium chloride (1M in THF, 0.45 mL, 0.45 mmol) was added to a solution of the compound of Preparation 49 (27 mg, 0.055 mmol) and the compound of Preparation 51 (37 mg, 0.11 mmol) in dry THF (2 mL) in a closed vial under nitrogen and stirred at room temperature for 1 hour. The reaction was quenched with MeOH (1 mL) and purified directly by prep. acidic HPLC to afford the desired compound as a mixture of SEM regioisomers (19 mg, 44% yield). LCMS (Method 3) (ES): m/z 778.8 [M+H]⁺, RT=1.20 min.

### Preparation 53: 4-[3,5-dimethyl-1-(2-trimethylsilylethoxymethyl)pyrazol-4-yl]-2-fluoro-aniline

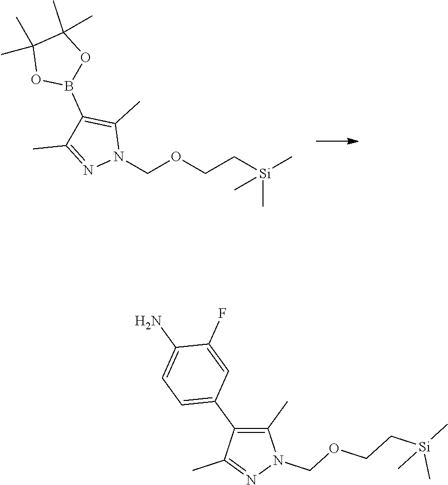

According to the method of Preparation 36 the compound of Preparation 50 (5.58 g, 15.87 mmol) was reacted with 5-bromo-6-fluoro-pyridin-2-amine (2.00 g, 10.58 mmol) to afford the title compound as an off-white solid (2.0 g, 57% yield). 1H NMR (300 MHz, CDCl₃) δ=6.89 (d, J=12.9 Hz, 1H), 6.83-6.80 (m, 2H), 5.37 (s, 2H), 3.74 (s, 2H), 3.62 (t, J=13.8 Hz, 2H), 2.28 (s, 3H), 2.22 (s, 3H), 0.93 (t, J=11.1 Hz, 2H), 0.01 (s, 9H); LCMS (Method 2) (ESI): m/z: 336 [M+H⁺]; 96%; RT=2.67 min (AQUITY BEH C18 column, 0.05% FA in water with MeCN).

### Preparation 54: SEM Protected 3,3-dicyclopropyl-N-[4-[3,5-dimethyl-1-(2-trimethylsilylethoxymethyl)pyrazol-4-yl]-2-fluoro-phenyl]-2-[5-(2-isopropylpyrazol-3-yl)-4H-1,2,4-triazol-3-yl]propanamide (Mixture of Isomers)

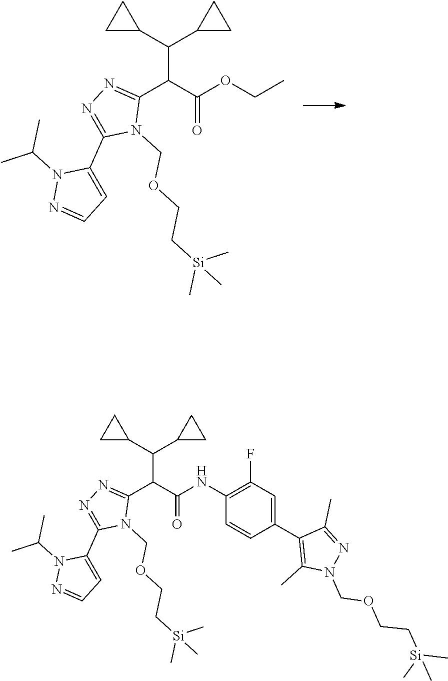

According to the method of Preparation 52, the compound of Preparation 49 (27 mg, 0.055 mmol) and the compound of Preparation 53 (24 mg, 0.072 mmol) were reacted to afford the title compound after prep. acidic HPLC as a mixture of SEM regioisomers (16 mg, 37% yield). LCMS (Method 3) (ES): m/z 777.8 [M+H]⁺, RT=1.22 min.

### Preparation 55: tert-butyl N-[(4-methyl-1,2,5-oxadiazole-3-carbonyl)amino]carbamate

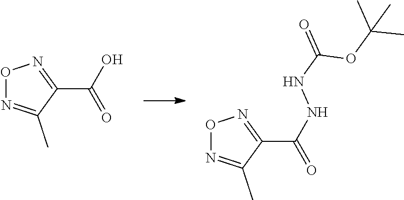

According to the method of Preparation 10 4-methyl-1,2,5-oxadiazole-3-carboxylic acid (1.0 g, 7.8 mmol) was reacted for 18 hours. The reaction mixture was diluted with water (30 mL) and extracted with Et₂O (2×40 mL). The combined organic phases was washed successively with water (20 mL), sodium hydrogen sulphate solution (10% in water, 20 mL) and saturated brine (20 mL), dried over MgSO₄, filtered and concentrated under reduced pressure. The concentrate was dissolved in DCM and purified by silica column chromatography (230-400 mesh), eluting with EtOAc (0-100%) in heptane giving the title compound (1.37 g, 72% yield). 1H NMR (400 MHz, CDCl₃) δ 8.42 (s, 1H), 6.56 (s, 1H), 2.62 (s, 3H), 1.50 (s, 9H); LCMS (Method 3) (ES): m/z 241.1 [M]⁻, RT=0.56 min.

### Preparation 56: 4-methyl-1,2,5-oxadiazole-3-carbohydrazide hydrochloride

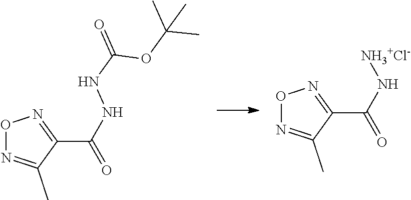

Hydrogen chloride (4M solution in 1,4-dioxane, 6 mL) was added to a solution of the compound of Preparation 55 (1.37 g, 5.66 mmol) in MeOH (2 mL) and stirred at room temperature for 1 hour, diluted with MeOH (5 mL) and concentrated in vacuo to leave the crude title compound (1.0 mg, 99% yield).

### Preparation 57: ethyl 2-[5-(4-methyl-1,2,5-oxadiazol-3-yl)-4H-1,2,4-triazol-3-yl]acetate

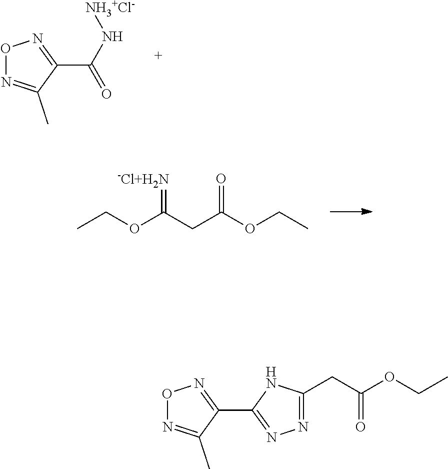

Ethyl 3-ethoxy-3-imino-propanoate hydrochloride (0.651 g, 2.83 mmol) and DIPEA (2.0 mL, 12 mmol) were added to a suspension of the compound of Preparation 56 (0.501 g, 2.83 mmol) in EtOH (8 mL) and stirred at 100° C. for 2 hours. The mixture was then concentrated under reduced pressure, dissolved in DCM and purified by silica column chromatography (230-400 mesh), eluting with a solvent mixture (5% Et₃N in EtOAc) (25-100%) in heptane, to afford the title compound (350 mg, 52% yield) as a yellow solid. 1H NMR (400 MHz, DMSO-d6) δ 14.60 (s, 1H), 4.15 (q, J=7.0 Hz, 2H), 4.05 (s, 2H), 2.64 (d, J=1.5 Hz, 3H), 1.22 (t, J=7.2, 3H); LCMS (Method 3) (ES): m/z 236.1 [M]⁻, RT=0.51 min.

### Preparation 58: ethyl 3-cyclopropyl-2-[5-(4-methyl-1,2,5-oxadiazol-3-yl)-4H-1,2,4-triazol-3-yl]prop-2-enoate (75:25 Mixture of Isomers)

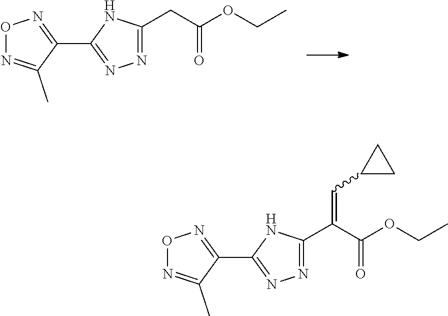 [SMILES: CCOC(=O)Cc1nnc(-c2nonc2C)[nH]1 ; CCOC(=O)C(=CC1CC1)c1nnc(-c2nonc2C)[nH]1]

Cyclopropanecarbaldehyde (0.057 mL, 0.77 mmol) was added to a solution of the compound of Preparation 57 (80 mg, 0.34 mmol), piperidine (0.1M in EtOH, 0.067 mL, 0.0067 mmol) in EtOH (3.4 mL) and the reaction mixture was stirred at 100° C. for 3 hours. The reaction was purified directly by prep. acidic HPLC to afford the desired compound (19 mg, 44% yield). 1H NMR (400 MHz, DMSO-d6) δ 14.58 (s, 1H, 75%), 14.35 (s, 1H, 25%) 6.76 (d,J=11.3 Hz, 1H, 75%), 6.59 (d, J=11.2 Hz, 1H, 25%), 4.32 (q, J=6.9 Hz, 2H, 25%), 4.22 (q, J=7.1 Hz, 2H, 75%), 2.66 (s, 3H, 75%), 2.64 (s, 3H, 25%), 1.29 (t, J=7.1 Hz, 3H, 25%), 1.24 (t, J=7.0 Hz, 3H, 75%), 1.15 (d, J=6.6 Hz, 2H, 25%), 1.09 (d, J=6.4, 2H, 75%), 0.94-0.86 (m, 2H, 75%, 2H 25%); LCMS (Method 3) (ES): m/z 288.2 [M]⁻, RT=0.71 min.

### Preparation 59: SEM Protected Ethyl 3-cyclopropyl-2-[5-(4-methyl-1,2,5-oxadiazol-3-yl)-4H-1,2,4-triazol-3-yl]prop-2-enoate (Mixture of Isomers)

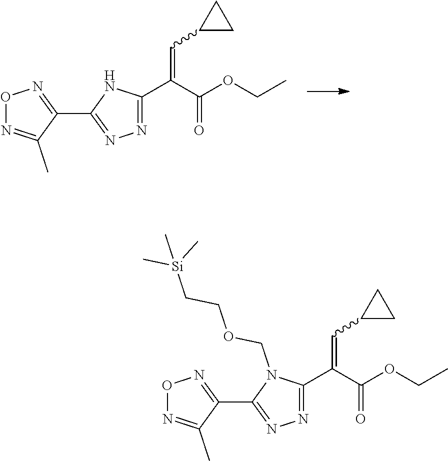

SEM chloride (0.191 mL, 1.08 mmol) was added to a solution of the compound of Preparation 58 (260 mg, 0.899 mmol) and potassium carbonate (248 mg, 1.80 mmol) in DMF (2.1 mL) under a nitrogen atmosphere and stirred for 1 hour at room temperature. The reaction mixture was diluted with Et₂O (10 mL) and water (5 mL) and separated. The aqueous phase was then extracted with Et₂O (5 mL). The combined organic phases were washed with water (2 mL), washed with brine, dried over MgSO₄, filtered and concentrated under reduced pressure giving the crude title compound as a mixture of SEM regioisomers (342 mg, 91% yield). LCMS (Method 3) (ES): m/z 420.3 [M+H]⁺, RT=0.92-0.99 min.

### Preparation 60: SEM Protected Ethyl 3,3-dicyclopropyl-2-[5-(4-methyl-1,2,5-oxadiazol-3-yl)-4H-1,2,4-triazol-3-yl]propanoate (Mixture of Isomers)

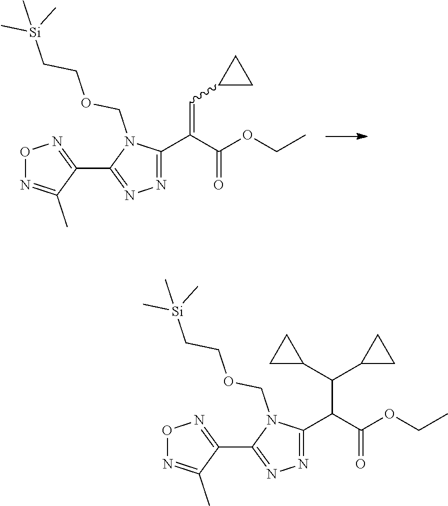

Bromo(cyclopropyl)magnesium (1M solution in 2-MeTHF, 1.8 mL, 1.8 mmol) was added dropwise to a pre-stirred solution of CuI (171 mg, 0.897 mmol) in anhydrous THF (5 mL) at 0° C. On complete addition the reaction mixture was stirred at 0° C. for 30 minutes, then cooled to −78° C. A solution of the compound of Preparation 59 (342 mg, 0.815 mmol) in anhydrous THF (5 mL) was then added dropwise and the reaction mixture was stirred at −78° C. for 30 minutes, then allowed to warm to room temperature for 3 hours. The reaction mixture was cooled to 0° C. and bromo(cyclopropyl)magnesium (1M solution in 2-MeTHF, 0.82 mL, 0.82 mmol) was added dropwise and stirred for 10 minutes before it was quenched with aq. NH₄Cl solution (20 mL) in Et₂O (100 mL) and the two phases were separated. The aqueous phase was extracted with Et₂O (50 mL). The combined organic phases were washed with water (20 mL) and saturated brine (20 mL), dried over MgSO₄, filtered and concentrated under reduced pressure. The residue was dissolved in DCM and purified by silica column chromatography (230-400 mesh), eluting with EtOAc (0-30%) in heptane giving the title compound as a mixture of SEM regioisomers (251 mg, 67% yield). LCMS (Method 3) (ES): m/z 462.3 [M+H]⁺, RT=1.04 and 1.07 min.

### Preparation 61: SEM Protected 3,3-dicyclopropyl-2-[5-(4-methyl-1,2,5-oxadiazol-3-yl)-4H-1,2,4-triazol-3-yl]propanoic acid (Mixture of Isomers)

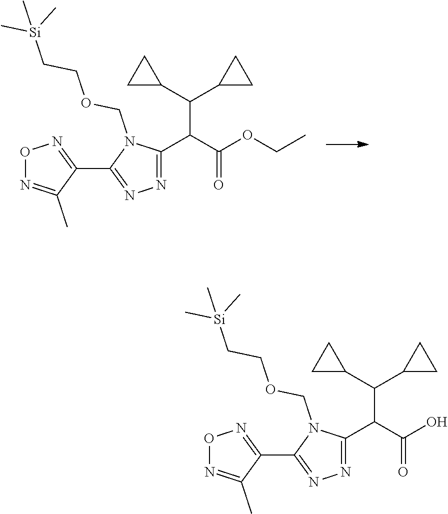

KOH (30 mg, 0.52 mmol) in water (0.40 mL) was added in portions to a solution of the product of Preparation 60 (60 mg, 0.13 mmol) in EtOH (1.0 mL) and stirred for 3 hours at room temperature. Et₂O (10 mL) and water (5 mL) were added to the reaction mixture and the pH was adjusted to 2-3 using a sodium hydrogen sulphate solution (10% in water). The two phases were separated and the aqueous phase was extracted with Et₂O (5 mL). The combined organic phases were washed with brine (5 mL), dried over MgSO₄, filtered and concentrated under reduced pressure giving the crude title compound as a mixture of SEM regioisomers (55 mg, 98% yield). LCMS (Method 3) (ES): m/z 434.3 [M+H]⁺, RT=0.92 min.

### Preparation 62: SEM Protected 3,3-dicyclopropyl-N-[4-[3,5-dimethyl-1-(2-trimethylsilylethoxymethyl)pyrazol-4-yl]phenyl]-2-[5-(4-methyl-1,2,5-oxadiazol-3-yl)-4H-1,2,4-triazol-3-yl]propanamide (Mixture of Isomers)

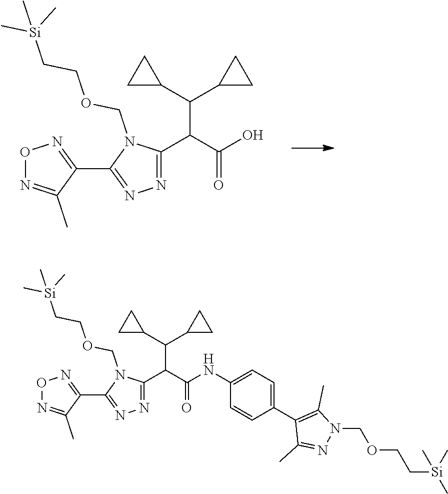

According to the method of Preparation 8, the compound of Preparation 61 (27 mg, 0.062 mmol) and the compound of Preparation 7 (20 mg, 0.062 mmol) were reacted for 15 minutes, diluted with Et₂O (10 mL) and water (3 mL) and separated. The water phase was extracted with Et₂O (5 mL), and the combined organic phases were washed with water (5 mL), washed with a sodium hydrogen sulphate solution (10% in water, 3 mL), washed with brine (3 mL), dried over MgSO₄, filtered and concentrated under reduced pressure giving the crude title compound as a mixture of SEM regioisomers (44 mg, 96% yield). LCMS (Method 3) (ES): m/z 733.5 [M+H]⁺, RT=1.12 min.

### Preparation 63: tert-butyl N-[(4-fluoro-2-methyl-pyrazole-3-carbonyl)amino]carbamate

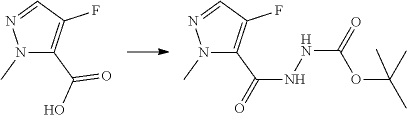

According to the method of Preparation 10 the compound of Preparation 32 (0.83 g, 5.8 mmol) was reacted for 30 minutes, then diluted with water (30 mL) and extracted with Et₂O (2×40 mL). The combined organic phase was washed successively with water (20 mL), sodium hydrogen sulphate solution (10% in water, 20 mL)and saturated brine (20 mL), dried over MgSO₄, filtered and concentrated under reduced pressure. The residue was dissolved in DCM and purified by silica column chromatography (230-400 mesh), eluting with EtOAc (0-100%) in heptane giving the title compound (1.14 g, 76% yield). 1H NMR (400 MHz, DMSO-d6) δ 9.96 (s, 1H), 9.03 (s, 1H), 7.59 (d, J=4.4 Hz, 1H), 3.91 (s, 3H), 1.43 (s, 9H); LCMS (Method 3) (ES): m/z 257.2 [M]⁻, RT=0.51 min.

### Preparation 64: 4-fluoro-2-methyl-pyrazole-3-carbohydrazide hydrochloride

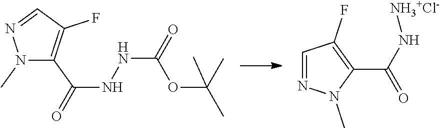

According to the method of Preparation 11 the compound of Preparation 63 (1.13 g, 4.38 mmol) was reacted for 30 minutes, diluted with MeOH (5 mL) and concentrated in vacuo to leave the crude title compound (0.85 g, assume 100% yield).

### Preparation 65: ethyl 2-[5-(4-fluoro-2-methyl-pyrazol-3-yl)-4H-1,2,4-triazol-3-yl]acetate

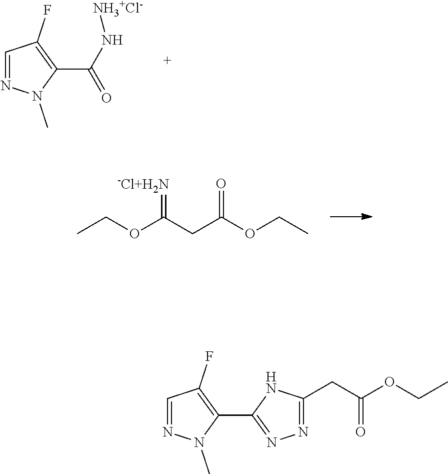

According to the method of Preparation 12, the compound of Preparation 64 (0.84 g, 4.3 mmol) was reacted for 12 hours in EtOH (10 mL). The mixture was then concentrated under reduced pressure, dissolved in DCM and purified by silica column chromatography (230-400 mesh), eluting with a solvent mixture (2% Et₃N in EtOAc, 25-100%) in heptane, to afford the title compound (350 mg, 52% yield) as a yellow solid. 1H NMR (400 MHz, DMSO-d6) δ 7.59 (d, J=4.3 Hz, 1H), 4.14 (q, J=7.1 Hz, 2H), 4.04 (d, J=0.8 Hz, 3H), 3.99 (s, 2H), 1.21 (t, J=7.1 Hz, 3H); LCMS (Method 3) (ES): m/z 252.1 [M]⁻, RT=0.45 min.

### Preparation 66: ethyl 3-cyclopropyl-2-[5-(4-fluoro-2-methyl-pyrazol-3-yl)-4H-1,2,4-triazol-3-yl]prop-2-enoate (Mixture of Isomers)

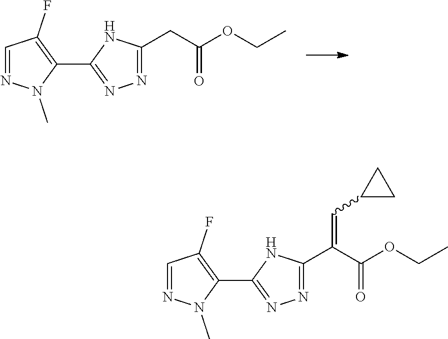 [SMILES: CCOC(=O)Cc1nnc(-c2c(F)cnn2C)[nH]1 ; CCOC(=O)C(=CC1CC1)c1nnc(-c2c(F)cnn2C)[nH]1]

According to the method of Preparation 58, the compound of Preparation 65 (0.53 g, 2.1 mmol) was reacted for 2 hours. The mixture was then concentrated under reduced pressure, recrystalized from Et₂O (5 mL) and filtered to afford the title compound (401 mg, 62% yield) as a yellow solid. 1H NMR (400 MHz, DMSO-d6) δ 14.31 (s, 1H), 7.59 (d, J=4.3 Hz, 1H), 6.71 (d, J=11.1 Hz, 1H), 4.21 (q, J=7.1 Hz, 2H), 4.07 (s, 3H), 2.23 (s, 1H), 1.23 (t, J=7.1 Hz, 3H), 1.13-0.77 (m, 4H); LCMS (Method 3) (ES): m/z 306.1 [M+H]⁺, RT=0.65 min.

### Preparation 67: SEM Protected Ethyl 3-cyclopropyl-2-[5-(4-fluoro-2-methyl-pyrazol-3-yl)-4H-1,2,4-triazol-3-yl]prop-2-enoate (Mixture of Isomers)

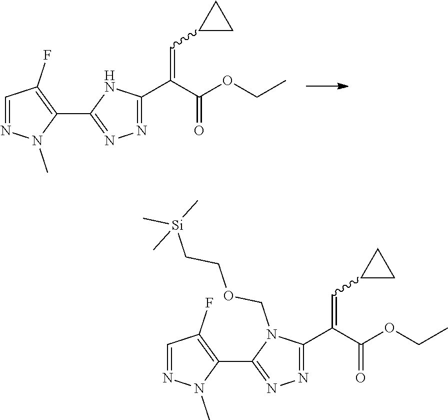

According to the method of Preparation 59, the compound of Preparation 66 (0.20 g, 0.66 mmol) was reacted for 1 hours to afford the title compound as a mixture of SEM regioisomers (261 mg, 91% yield). LCMS (Method 3) (ES): m/z 436.3 [M+H]⁺, RT=0.92-0.94 min.

### Preparation 68: SEM Protected Ethyl 3,3-dicyclopropyl-2-[5-(4-fluoro-2-methyl-pyrazol-3-yl)-4H-1,2,4-triazol-3-yl]propanoate (Mixture of Isomers)

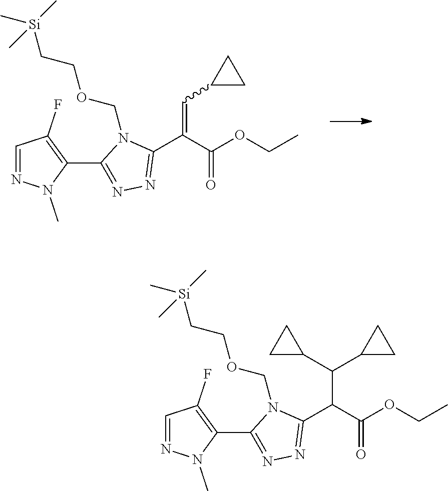

According to the method of Preparation 60, the compound of Preparation 67 (0.26 g, 0.60 mmol) was reacted to afford the title compound as a mixture of SEM regioisomers (158 mg, 55% yield). LCMS (Method 3) (ES): m/z 478.3 [M+H]⁺, RT=1.02 min.

### Preparation 69: SEM Protected 3,3-dicyclopropyl-2-[5-(4-fluoro-2-methyl-pyrazol-3-yl)-4H-1,2,4-triazol-3-yl]propanoic acid (Mixture of Isomers)

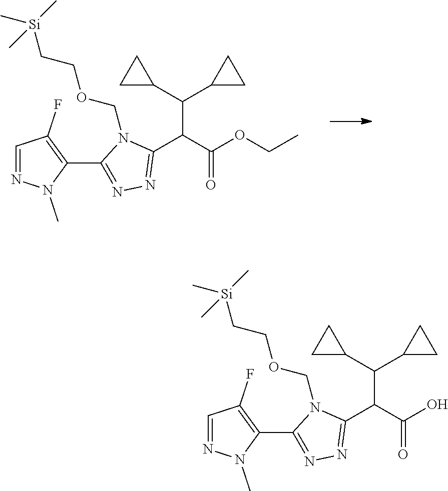

According to the method of Preparation 61, the compound of Preparation 68 (64 mg, 0.13 mmol) was reacted to afford the title compound as a mixture of SEM regioisomers (59 mg, 98% yield). LCMS (Method 3) (ES): m/z 450.3 [M+H]⁺, RT=0.89 min.

### Preparation 70: SEM Protected 3,3-dicyclopropyl-N-[4-[3,5-dimethyl-1-(2-trimethylsilylethoxymethyl)pyrazol-4-yl]phenyl]-2-[5-(4-fluoro-2-methyl-pyrazol-3-yl)-4H-1,2,4-triazol-3-yl]propanamide (Mixture of Isomers)

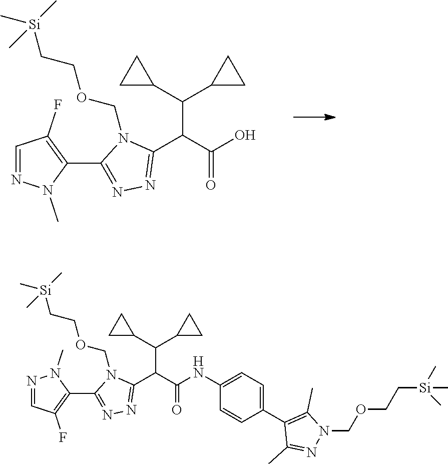

According to the method of Preparation 8, the compound of Preparation 69 (29 mg, 0.065 mmol) and the compound of Preparation 7 (20 mg, 0.065 mmol) were reacted for 15 minutes and purified directly by prep. acidic HPLC to afford the title compound as a mixture of SEM regioisomers (36 mg, 75% yield). LCMS (Method 3) (ES): m/z 749.5 [M+H]⁺, RT=1.12 min.

### Preparation 71: ethyl 2-(5-benzyl-4H-1,2,4-triazol-3-yl)acetate

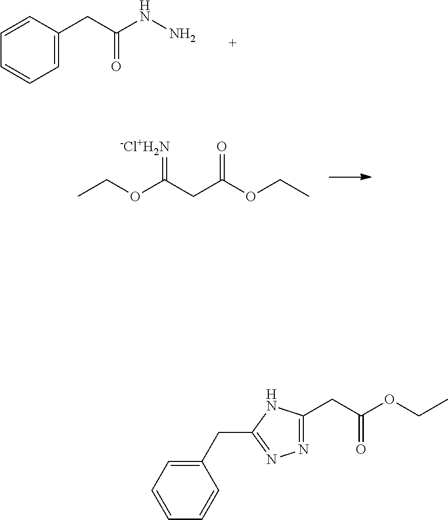

Ethyl 3-ethoxy-3-imino-propanoate hydrochloride (3.68 g, 16.0 mmol) and DIPEA (2.78 mL, 16.0 mmol) were dissolved in MeCN (20 mL) and stirred at room temperature for 30 minutes. 2-Phenylacetohydrazide (2.00 g, 13.3 mmol) and AcOH (0.38 mL, 6.6 mmol) were added and the reaction mixture was stirred at 100° C. for 18 hours. The mixture was then concentrated under reduced pressure, dissolved in DCM and purified by silica column chromatography (230-400 mesh), eluting with a solvent mixture (2% Et₃N in EtOAc, 25-100%) in heptane, to afford the title compound (1.31 g, 40% yield) as a yellow solid. 1H NMR (400 MHz, DMSO-d6) δ 13.57 (s, 1H), 7.35-7.16 (m, 5H), 4.08 (q, J=7.1 Hz, 2H), 4.00 (s, 2H), 3.71 (s, 2H), 1.17 (t, J=7.1 Hz, 3H); LCMS (Method 3) (ES): m/z 246.2 [M+H]⁺, RT=0.50 min.

### Preparation 72: ethyl 2-(5-benzyl-4H-1,2,4-triazol-3-yl)-3-cyclopropyl-prop-2-enoate (80:20 Mixture of Isomers)

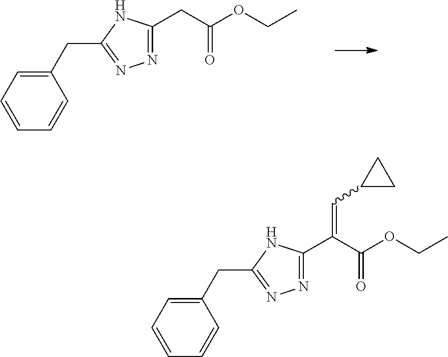 [SMILES: CCOC(=O)Cc1nnc(Cc2ccccc2)[nH]1 ; CCOC(=O)C(=CC1CC1)c1nnc(Cc2ccccc2)[nH]1]

According to the method of Preparation 58, the compound of Preparation 71 (0.50 g, 1.63 mmol) was reacted for 2 hours. The mixture was then concentrated under reduced pressure, recrystalized from Et₂O (5 mL) and filtered to afford the title compound (456 mg, 62% yield) as a yellow solid. 1H NMR (400 MHz, CDCl₃) δ 11.65 (s, 1H, 80%, 1H 20%), 7.39-7.16 (m, 5H 80%, 5H 20%), 6.93 (d, J=11.4 Hz, 1H, 20%), 6.62 (d, J=11.3 Hz, 1H, 80%), 4.40 (q, J=7.1 Hz, 2H, 20%), 4.28 (q, J=7.1 Hz, 2H, 80%), 4.12 (s, 2H, 80%), 4.07 (s, 2H, 20%), 3.50-3.39 (m, 1H, 80%), 2.75-2.66 (m, 1H, 20%), 1.40 (t, J=7.1 Hz, 3H, 20%), 1.33 (t, J=7.1 Hz, 3H, 80%), 1.30-0.79 (m, 4H, 80%, 4H, 20%); LCMS (Method 3) (ES): m/z 298.2 [M+H]⁺, RT=0.65 and 0.67 min.

### Preparation 73: SEM Protected Ethyl 2-(5-benzyl-4H-1,2,4-triazol-3-yl)-3-cyclopropyl-prop-2-enoate (Mixture of Isomers)

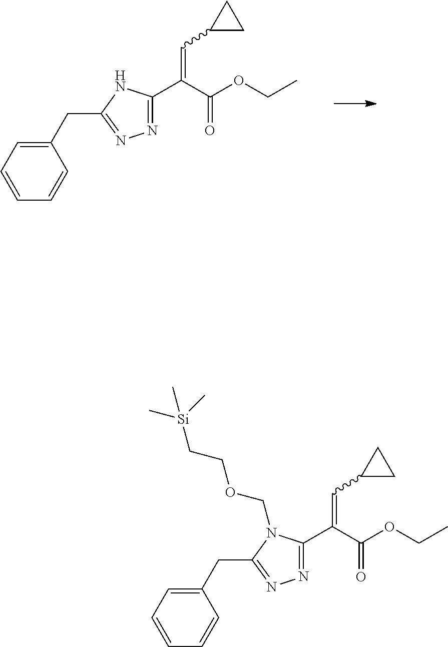

According to the method of Preparation 59, the compound of Preparation 72 (0.36 g, 1.2 mmol) was reacted with SEM chloride (0.36 mL, 2.1 mmol) added in two portions for 1 hour to afford the title compound as a mixture of SEM regioisomers (0.50 g, 96% yield). LCMS (Method 3) (ES): m/z 428.3 [M+H]⁺, RT=0.95-0.97 min.

### Preparation 74: SEM Protected Ethyl 2-(5-benzyl-4H-1,2,4-triazol-3-yl)-3,3-dicyclopropyl-propanoate (Mixture of Isomers)

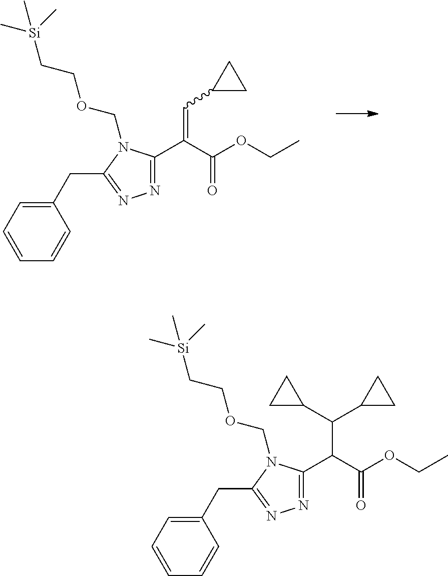

According to the method of Preparation 60, the compound of Preparation 73 (490 mg, 1.15 mmol) was reacted to afford the title compound as a mixture of SEM regioisomers (221 mg, 41% yield). LCMS (Method 3) (ES): m/z 470.4 [M+H]⁺, RT=1.03 and 1.04 min.

### Preparation 75: SEM Protected 2-(5-benzyl-4H-1,2,4-triazol-3-yl)-3,3-dicyclopropyl-propanoic acid (Mixture of Isomers)

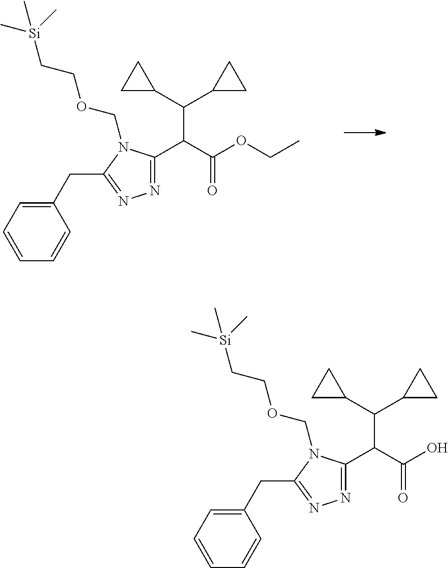

According to the method of Preparation 61, the compound of Preparation 74 (64 mg, 0.13 mmol) was reacted for 3 days to afford the title compound as a mixture of SEM regioisomers (59 mg, 98% yield). LCMS (Method 3) (ES): m/z 440.3 [M]⁻, RT=0.93 min.

### Preparation 76: SEM Protected 2-(5-benzyl-4H-1,2,4-triazol-3-yl)-3,3-dicyclopropyl-N-[4-[3,5-dimethyl-1-(2-trimethylsilylethoxymethyl)pyrazol-4-yl]phenyl]propanamide (Mixture of Isomers)

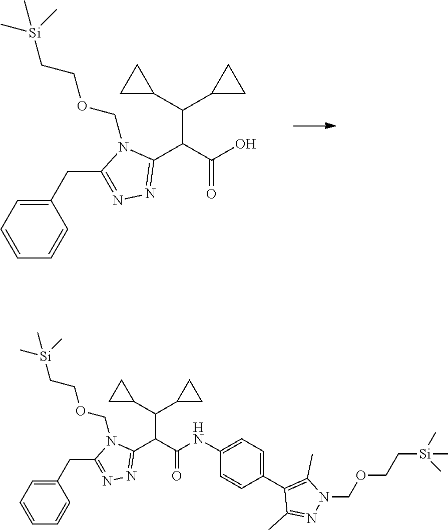

According to the method of Preparation 8, the compound of Preparation 75 (81 mg, 0.18 mmol) and the compound of Preparation 7 (58 mg, 0.18 mmol) were reacted for 1 hour and purified directly by prep. acidic HPLC to afford the title compound as a mixture of SEM regioisomers (44 mg, 32% yield). LCMS (Method 3) (ES): m/z 741.6 [M+H]⁺, RT=1.15 and 1.17 min.

### Preparation 77: ethyl 2-(3-pyridyl)ethanimidate dihydrochloride

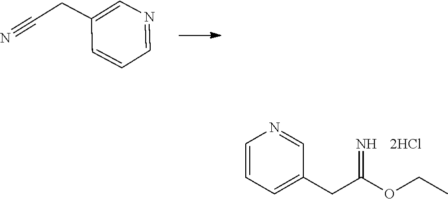

HCl (4M in dioxane, 20 mL, 80 mmol) was added to a solution of 2-(3-pyridyl)acetonitrile (2.15 g, 18.2 mmol) in EtOH (dried over mol·sieves (3 Å), 1.4 mL) and stirred for 18 hours at room temperature, giving a two phase mixture. The dioxane phase was decanted off, and the remaining oily residue was concentrated twice from Et₂O under reduced pressure to afford the crude title compound (4.67 g, assume 100% yield).

### Preparation 78: 3,3-dicyclopropyl-N-[4-[3,5-dimethyl-1-(2-trimethylsilylethoxymethyl)pyrazol-4-yl]phenyl]-2-[5-(3-pyridylmethyl)-4H-1,2,4-triazol-3-yl]propanamide

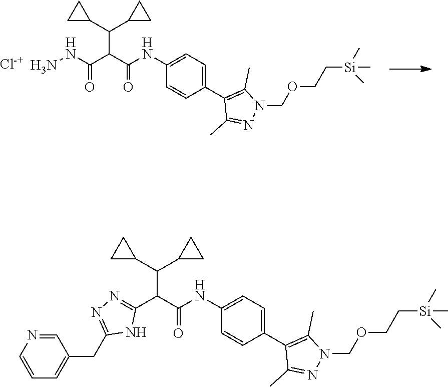

TEA (0.25 mL, 1.8 mmol) was added to a solution of the compound of Preparation 11 (0.10 g, 0.18 mmol) and the compound of Preparation 77 (130 mg, 0.55 mmol) added in two portions in EtOH (2 mL) and stirred at 100° C. for 18 hours. The reaction mixture was diluted with MeOH (2 mL) and purified directly by prep. basic HPLC to afford the title compound (69 mg, 62% yield). LCMS (Method 4) (ES): m/z 610.4 [M]⁻, RT=0.81 min.

### Preparation 79: ethyl 2-(1H-pyrazol-3-yl)ethanimidate dihydrochloride

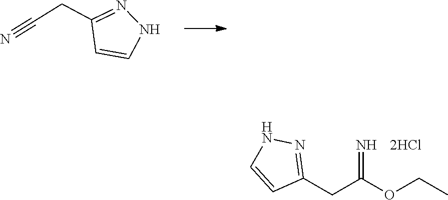

HCl (4M in dioxane, 4 mL, 16 mmol) was added to a solution of 2-(1H-pyrazol-3-yl)acetonitrile (0.20 g, 1.9 mmol) in EtOH (dried over mol·sieves (3 Å), 0.14 mL) and stirred for 18 hours at room temperature giving a slurry. The reaction mixture was dried under reduced pressure to afford the crude title compound (0.42 mg, assume 100% yield).

### Preparation 80: 3,3-dicyclopropyl-N-[4-[3,5-dimethyl-1-(2-trimethylsilylethoxymethyl)pyrazol-4-yl]phenyl]-2-[5-(1H-pyrazol-3-ylmethyl)-4H-1,2,4-triazol-3-yl]propanamide

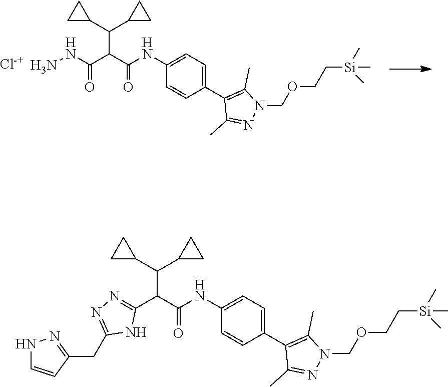

According to the method of Preparation 78, the compound of Preparation 11 (36 mg, 0.065 mmol) and the compound of Preparation 79 (65 mg, 0.13 mmol) were reacted to afford the title compound after prep. basic HPLC (18 mg, 46% yield). LCMS (Method 4) (ES): m/z 601.5 [M+H]⁺, RT=0.79 min.

### Preparation 81: dimethyl 2-(4,4-difluorocyclohexylidene)propanedioate

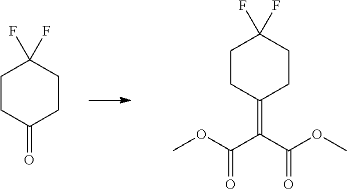

4,4-Difluorocyclohexanone (5.0 g, 37 mmol), dimethyl malonate (4.7 mL, 41 mmol) and pyridine (12 mL, 0.15 mol) were added to a solution of TiCl₄ (8.2 mL, 75 mmol) in a mixture of DCM (10 mL) and THF (30 mL) at 0° C. The reaction mixture was allowed to warm to room temperature and stirred for 16 hours. The reaction mixture was diluted with EtOAc (100 mL), washed with water (2×250 mL), brine (100 mL), dried over Na₂SO₄, filtered and concentrated under reduced pressure. The concentrate was dissolved in EtOAc and purified by silica column chromatography (230-400 mesh), eluting with EtOAc (10%) in hexane, to afford the title compound (3.5 g, 38% yield) as a brown oil. 1H NMR (400 MHz, CDCl₃) δ 3.78 (s, 6H), 2.74-2.71 (m, 4H), 2.13-2.03 (m, 4H); LCMS (Method 2) (ES): m/z 249 [M+H]⁺, RT=2.05 min.

### Preparation 82: dimethyl 2-(4,4-difluorocyclohexyl)propanedioate

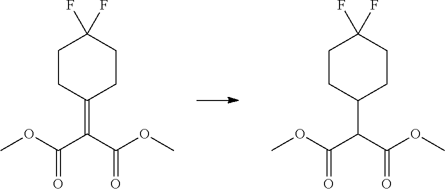

10% Pd/C (40 g) was added to a solution of the compound of Preparation 81 (180 g, 0.726 mol) in EtOH (500 mL) and stirred under 70 psi hydrogen in an autoclave for 16 hours at room temperature. The reaction mixture was then filtered through celite and concentrated under reduced pressure to afford the crude title compound (160 g, 88%) as a brown solid. 1H NMR (400 MHz, CDCl₃) δ 3.75 (s, 6H), 3.25 (d, J=9.2 Hz, 1H), 2.16-2.07 (m, 3H), 1.82-1.71 (m, 4H), 1.43-1.39 (m, 2H); LCMS (Method 2) (ES): m/z 251 [M+H]⁺, RT=1.86.

### Preparation 83: Mixture of 2-(4,4-difluorocyclohexyl)-3-methoxy-3-oxo-propanoic acid and 2-(4,4-difluorocyclohexyl)-3-ethoxy-3-oxo-propanoic acid

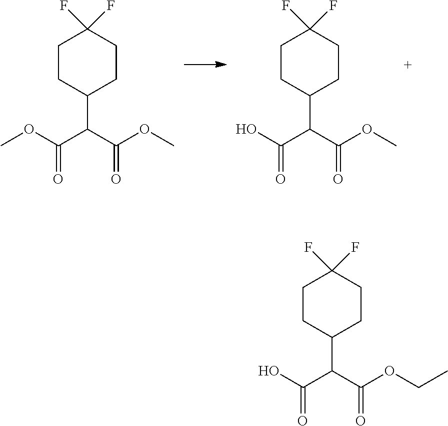

NaOH (25 g, 0.64 mol) was added to a solution of the compound of Preparation 82 (160 g, 0.640 mol) in EtOH (342 mL) at 0° C. and refluxed at 16 hours. The reaction mixture was concentrated under reduced pressure. The residue was diluted with water (300 mL) and washed with EtOAc (2×500 mL). The aqueous phase was acidified to pH ˜2 using HCl (1M) and extracted with EtOAc (2×500 mL). The mixture was then concentrated under reduced pressure and purified by silica column chromatography (230-400 mesh), eluting with EtOAc (40%) in petroleum ether, to afford the title compounds (68 g, 42% yield) as an off white solid. LCMS (Method 2) (ES): m/z 235 [M]⁻, RT=1.61 min and m/z 249 [M]⁻, RT=1.72 min.

### Preparation 84: Mixture of methyl 3-amino-2-(4,4-difluorocyclohexyl)-3-oxo-propanoate and ethyl 3-amino-2-(4,4-difluorocyclohexyl)-3-oxo-propanoate

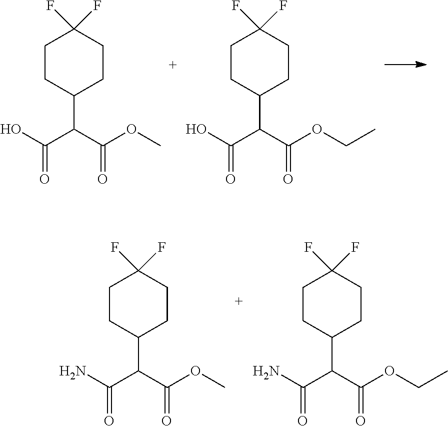

Ammonia (25% in water, 30 mL) and CDI (49 g, 0.31 mol) were added to a solution of the compounds of Preparation 83 (48 g, 0.20 mol) in THF (50 mL) at 0° C. and stirred at room temperature for 16 hours. The reaction mixture was diluted with water (200 mL) and washed with EtOAc (2×200 mL). The aqueous phase was acidified to pH ˜2 using HCl (1M) and extracted with EtOAc (2×200 mL), dried over Na₂SO₄, filtered and concentrated under reduced pressure giving the crude title compounds (35 g, 74% yield) as a colourless oil. LCMS (Method 2) (ES): m/z 236 [M+H]⁺, RT=1.44 min and m/z 250 [M+H]⁺, RT=1.56 min.

### Preparation 85: Mixture of methyl 2-(4,4-difluorocyclohexyl)-3-[(E)-dimethylaminomethyleneamino]-3-oxo-propanoate and ethyl 2-(4,4-difluorocyclohexyl)-3-[(E)-dimethylaminomethyleneamino]-3-oxo-propanoate

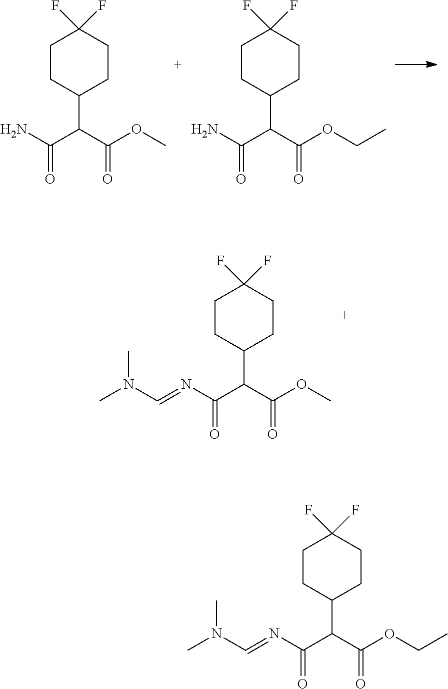

DMF·DMA (21 mL, 0.17 mol) was added to a solution of the compounds of Preparation 84 (20 g, 85 mmol) in DCM (50 mL) at 0° C. and stirred at 90° C. for 2 hours. The reaction mixture was diluted with water (200 mL), extracted with EtOAc (2×200 mL), dried over Na₂SO₄, filtered and concentrated under reduced pressure giving the crude title compounds (23 g, 93% yield) as a brown oil. LCMS (Method 2) (ES): m/z 291 [M+H]⁺, RT=1.37 min and m/z 305 [M+H]⁺, RT=1.68 min.

### Preparation 86: Mixture of methyl 2-(4,4-difluorocyclohexyl)-2-(4H-1,2,4-triazol-3-yl)acetate and ethyl 2-(4,4-difluorocyclohexyl)-2-(4H-1,2,4-triazol-3-yl)acetate

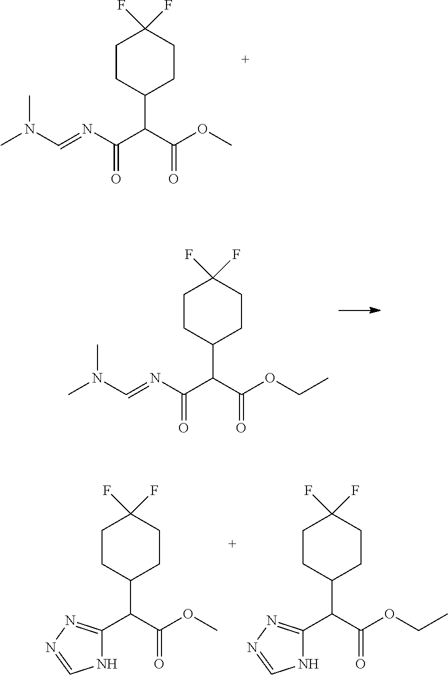

Hydrazine monohydrate (38 mL, 0.79 mmol) was added to a solution of the compounds of Preparation 85 (23 g, 79 mmol) in AcOH (45 mL) at 0° C. and stirred at room temperature for 16 hours. The reaction mixture was concentrated under reduced pressure, diluted with water (100 mL) and extracted with EtOAc (2×100 mL). The combined organic phase was washed with aq. sodium hydrogen carbonate solution (200 mL), dried over Na₂SO₄, filtered and concentrated under reduced pressure giving the crude title compound (10 g, 50% yield) as a brown oil. LCMS (Method 2) (ES): m/z 260 and 274 [M+H]⁺, RT=1.42 min.

### Preparation 87: methyl 2-(5-bromo-4H-1,2,4-triazol-3-yl)-2-(4,4-difluorocyclohexyl)acetate

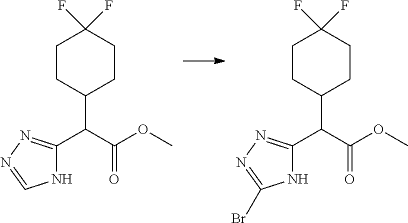 [SMILES: COC(=O)C(c1nnc[nH]1)C1CCC(F)(F)CC1 ; COC(=O)C(c1nnc(Br)[nH]1)C1CCC(F)(F)CC1]

NBS (14 g, 77 mmol) was added to a solution of the compound of Preparation 86 (10 g, 39 mmol) in MeCN (90 mL) at 0° C. and stirred at 100° C. for 3 hours. The reaction mixture was diluted with water (150 mL) and extracted with EtOAc (2×200 mL). The combined organic phase was washed with a sodium hydrogen sulphate solution (in water, 200 mL), dried over Na₂SO₄, filtered and concentrated under reduced pressure giving the crude title compound (7.2 g, 55% yield) as a pale brown solid. 1H NMR (400 MHz, DMSO-d6) δ 14.52-14.39 (m, 1H), 3.85 (d, J=8.7 Hz, 1H), 3.65 (s, 3H), 2.32-2.16 (m, 1H), 2.08-1.88 (m, 2H), 1.86-1.70 (m, 3H), 1.51-1.40 (m, 1H), 1.33-1.20 (m, 2H); LCMS (Method 2) (ES): m/z 388 [M+H]⁺, RT=1.66 min.

### Preparation 88: SEM Protected methyl 2-(5-bromo-4H-1,2,4-triazol-3-yl)-2-(4,4-difluorocyclohexyl)acetate (Mixture of Isomers)

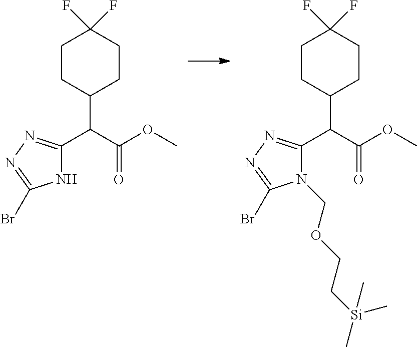 [SMILES: COC(=O)C(c1nnc(Br)[nH]1)C1CCC(F)(F)CC1 ; COC(=O)C(c1nnc(Br)n1COCC[Si](C)(C)C)C1CCC(F)(F)CC1]

Potassium carbonate (2.0 g, 15 mmol) was added to a solution of the compound of Preparation 87 (2.5 g, 7.4 mmol) in MeCN (30 mL) at 0° C. SEM chloride (1.7 mL, 9.6 mmol) was then added and the reaction mixture stirred at room temperature for 1 hour. The reaction mixture was diluted with water, extracted with EtOAc (2×50 mL), washed with brine, dried over Na₂SO₄, filtered and concentrated under reduced pressure giving the crude title compound as a mixture of SEM regioisomers (3.2 g, 92% yield) as a yellow solid. LCMS (Method 2) (ES): m/z 468 [M+H]⁺, RT=2.41 and 2.45 min.

### Preparation 89: SEM Protected 2-(5-bromo-4H-1,2,4-triazol-3-yl)-2-(4,4-difluorocyclohexyl)acetic acid (Mixture of Isomers)

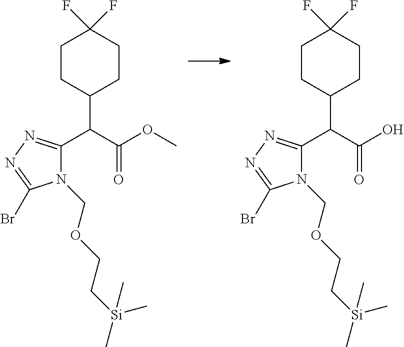 [SMILES: COC(=O)C(c1nnc(Br)n1COCC[Si](C)(C)C)C1CCC(F)(F)CC1 ; C[Si](C)(C)CCOCn1c(Br)nnc1C(C(=O)O)C1CCC(F)(F)CC1]

LiOH monohydrate (3.1 g, 75 mmol) was added to a solution of the compound of Preparation 88 (3.5 g, 7.5 mmol) in a mixture of THF (20 mL) and water (10 mL) and stirred at room temperature for 4 hours. The reaction mixture was concentrated under reduced pressure, and the concentrate was acidified with a citric acid solution (10% in water, 100 mL), extracted with EtOAc (150 mL), dried over Na₂SO₄, filtered and concentrated under reduced pressure giving the crude title compound (2.3 g, 69% yield) as a pale yellow solid as a mixture of SEM regioisomers. LCMS (Method 2) (ES): m/z 454 [M+H]⁺, RT=2.20 and 2.25 min.

### Preparation 90: SEM Protected 2-(5-bromo-4H-1,2,4-triazol-3-yl)-2-(4,4-difluorocyclohexyl)-N-[4-[3,5-dimethyl-1-(2-trimethylsilylethoxymethyl)pyrazol-4-yl]phenyl]acetamide (Mixture of Isomers)

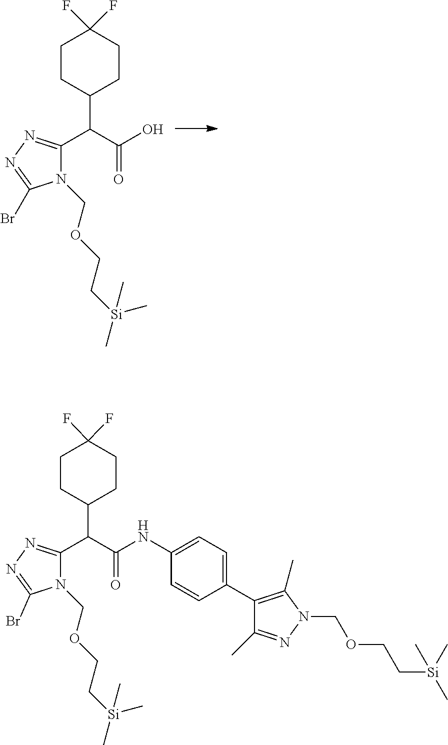 [SMILES: C[Si](C)(C)CCOCn1c(Br)nnc1C(C(=O)O)C1CCC(F)(F)CC1 ; Cc1nn(COCC[Si](C)(C)C)c(C)c1-c1ccc(NC(=O)C(c2nnc(Br)n2COCC[Si](C)(C)C)C2CCC(F)(F)CC2)cc1]

DIPEA (1.4 mL, 7.9 mmol) and HATU (1.5 g, 4.0 mmol) were added to a solution of the compound of Preparation 89 (1.2 g, 2.6 mmol) and the compound of Preparation 7 (0.67 g, 2.1 mmol) in DMF (12 mL) at 0° C. and stirred at room temperature for 30 minutes. The reaction mixture was diluted with ice water and extracted with EtOAc (2×50 mL). The combined organic phase was dried over Na₂SO₄, filtered and concentrated under reduced pressure. The concentrate was dissolved in DCM and purified by silica column chromatography (230-400 mesh), eluting with EtOAc (0-100%) in hexane, to afford the title compound (0.80 g, 40% yield) as a yellow oil as a mixture of SEM regioisomers. LCMS (Method 2) (ES): m/z 753 [M+H]⁺, RT=2.71 min and 2.74 min.

### Preparation 91: 3,3-dicyclopropyl-N-[4-[3,5-dimethyl-1-(2-trimethylsilylethoxymethyl)pyrazol-4-yl]phenyl]-2-(1H-imidazol-2-yl)propanamide

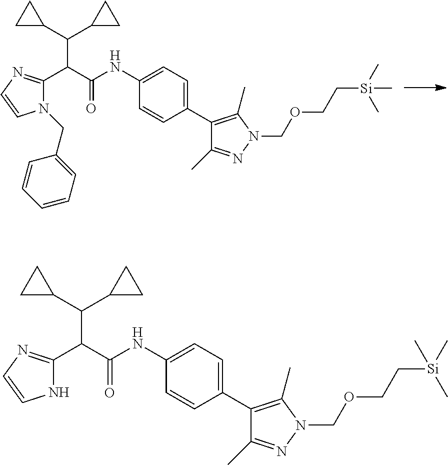 [SMILES: Cc1nn(COCC[Si](C)(C)C)c(C)c1-c1ccc(NC(=O)C(c2nccn2Cc2ccccc2)C(C2CC2)C2CC2)cc1 ; Cc1nn(COCC[Si](C)(C)C)c(C)c1-c1ccc(NC(=O)C(c2ncc[nH]2)C(C2CC2)C2CC2)cc1]

10% Pd/C (100 mg) was added to a nitrogen purged solution of the compound of Preparation 25 (700 mg, 1.03 mmol) in MeOH (10 mL) under balloon pressure of hydrogen and stirred at room temperature for 2.5 hours. The reaction mixture was diluted with MeOH (4 mL), filtered through Celite™, washing the pad with hot MeOH:DMF (4:1), and concentrated in vacuo to give the title compound (537 mg, assume 100% yield). LCMS (Method 4) (ES): m/z 520.3 [M+H]⁺, RT=0.86 min.

### Preparation 92: 3,3-dicyclopropyl-N-[4-[3,5-dimethyl-1-(2-trimethylsilylethoxymethyl)pyrazol-4-yl]phenyl]-2-[1-(2-trimethylsilylethoxymethyl)imidazol-2-yl]propanamide

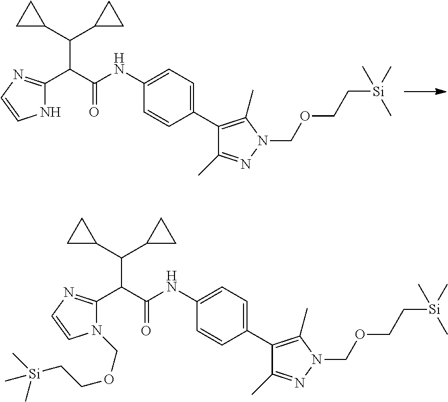

According to the method of Preparation 59, the compound of Preparation 91 (537 mg, 1.03 mmol) was reacted for 16 hours to afford the title compound after flash silica chromatography, eluting with EtOAc in heptane (167 mg, 25% yield). LCMS (Method 4) (ES): m/z 650.4 [M+H]⁺, RT=1.05 min.

### Preparation 93: SEM Protected 2-(4-bromo-1H-imidazol-2-yl)-3,3-dicyclopropyl-N-[4-[3,5-dimethyl-1-(2-trimethylsilylethoxymethyl)pyrazol-4-yl]phenyl]propanamide

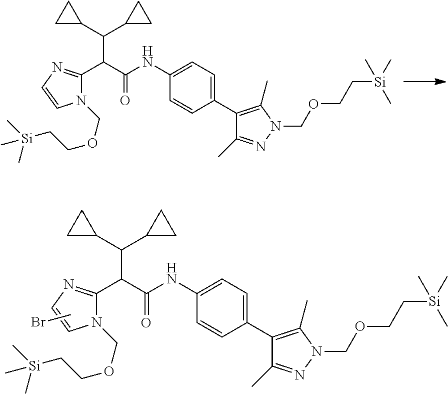

NBS (41.0 mg, 0.231 mmol) was added to a solution of the compound of Preparation 92 (150 mg, 0,231 mmol) in DCM (5 mL) and stirred at room temperature for 1 hour. A further portion of NBS (14.4 MG, 0.081 mmol) was added and the reaction mixture was stirred for a further 1 hour. The reaction mixture was diluted with DCM (20 mL), washed with H₂O (2×5 mL), saturated brine solution (5 mL), dried over MgSO₄, filtered and concentrated in vacuo, to give the title compound (168 mg, assume 100% yield). LCMS (Method 4) (ES): m/z 672.3 [M+H]⁺, RT=1.09 min.

### EXAMPLES

### Example 1: 3,3-dicyclopropyl-N-[4-(3,5-dimethyl-1H-pyrazol-4-yl)phenyl]-2-(5-phenyl-4H-1,2,4-triazol-3-yl)propanamide

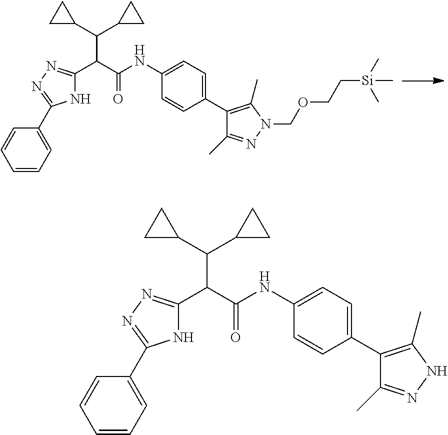 [SMILES: Cc1nn(COCC[Si](C)(C)C)c(C)c1-c1ccc(NC(=O)C(c2nnc(-c3ccccc3)[nH]2)C(C2CC2)C2CC2)cc1 ; Cc1n[nH]c(C)c1-c1ccc(NC(=O)C(c2nnc(-c3ccccc3)[nH]2)C(C2CC2)C2CC2)cc1]

TFA (1 mL) was added to a solution of the compound of Preparation 12 (7.0 mg, 0.012 mmol) in DCM (1 mL) and stirred at room temperature for 1 hour. The reaction mixture was concentrated in vacuo, re-dissolved in MeOH (2 mL) and purified directly by prep. acidic HPLC to afford the title compound (8.5 mg, 63% yield). 1H NMR (400 MHz, DMSO-d6) δ 13.88 (s, 1H), 11.78 (s, 1H), 10.39 (s, 1H), 8.05-7.95 (m, 2H), 7.64 (d, J=8.4 Hz, 2H), 7.50-7.35 (m, 3H), 7.22 (d, J=8.4 Hz, 2H), 4.20 (d, J=9.7 Hz, 1H), 2.17 (s, 6H), 1.23 (dd, J=11.2, 7.4 Hz, 1H), 0.92-0.74 (m, 1H), 0.74-0.59 (m, 1H), 0.48-0.40 (m, 1H), 0.39-0.17 (m, 5H), 0.10-0.02 (m, 1H), −0.02-−0.12 (m, 1H); LCMS (ES): m/z 467.256 [M+H]⁺, RT=2.30 min.

### Example 2: 3,3-dicyclopropyl-N-[4-(3,5-dimethyl-1H-pyrazol-4-yl)phenyl]-2-(4-phenyl-1H-imidazol-2-yl)propanamide

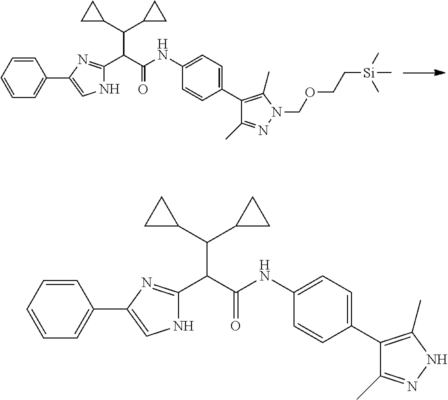 [SMILES: Cc1nn(COCC[Si](C)(C)C)c(C)c1-c1ccc(NC(=O)C(c2nc(-c3ccccc3)c[nH]2)C(C2CC2)C2CC2)cc1 ; Cc1n[nH]c(C)c1-c1ccc(NC(=O)C(c2nc(-c3ccccc3)c[nH]2)C(C2CC2)C2CC2)cc1]

According to the method of Example 1 the compound of Preparation 14 (10 mg, 0.022 mmol) was reacted to afford the title compound after prep. basic HPLC (4.0 mg, 51% yield). 1H NMR (600 MHz, DMSO-d6) δ 12.22 (s, 1H), 12.15-11.78 (m, 1H), 10.55-10.30 (m, 1H), 7.83-7.10 (m, 10H), 4.15-3.93 (m, 1H), 2.17 (s, 6H), 1.35-1.03 (m, 1H), 0.83-0.56 (m, 2H), 0.48-0.02 (m, 7H), −0.06-−0.14 (m, 1H); LCMS (ES): m/z 466.261 [M+H]⁺, RT=2.06 min.

### Example 3: 2-(5-chloro-4-phenyl-1H-imidazol-2-yl)-3,3-dicyclopropyl-N-[4-(3,5-dimethyl-1H-pyrazol-4-yl)phenyl]propanamide

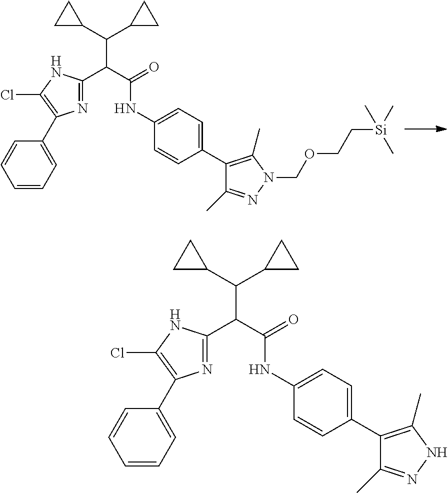

According to the method of Example 1 the compound of Preparation 15 (5.0 mg, 0.008 mmol) was reacted to afford the title compound after prep. basic HPLC (3.0 mg, 75% yield). 1H NMR (600 MHz, DMSO-d6) δ 12.37 (s, 1H), 12.22 (s, 1H), 10.28 (s, 1H), 7.74 (d, J=7.8 Hz, 2H), 7.68-7.57 (m, 2H), 7.46 (t, J=7.6 Hz, 2H), 7.32 (d, J=7.8 Hz, 1H), 7.26-7.16 (m, 2H), 4.15-3.77 (m, 1H), 2.17 (d, J=23.6 Hz, 6H), 1.34 (d, J=9.9 Hz, 1H), 0.84-0.74 (m, 1H), 0.66 (s, 1H), 0.45 (dp, J=9.1, 4.6, 4.1 Hz, 1H), 0.39-0.15 (m, 5H), 0.10 (td, J=9.1, 4.6 Hz, 1H), −0.05 (s, 1H).

### Example 4: 3,3-dicyclopropyl-N-[4-(3,5-dimethyl-1H-pyrazol-4-yl)phenyl]-2-(4H-1,2,4-triazol-3-yl)propanamide

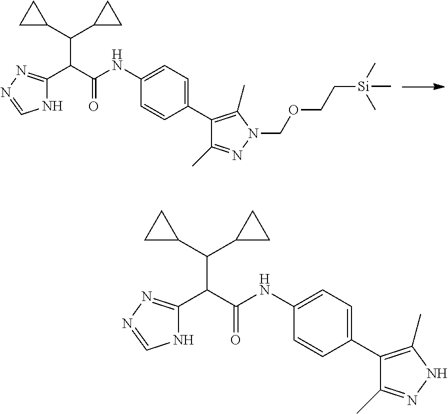 [SMILES: Cc1nn(COCC[Si](C)(C)C)c(C)c1-c1ccc(NC(=O)C(c2nnc[nH]2)C(C2CC2)C2CC2)cc1 ; Cc1n[nH]c(C)c1-c1ccc(NC(=O)C(c2nnc[nH]2)C(C2CC2)C2CC2)cc1]

According to the method of Example 1 the compound of Preparation 17 (45 mg, 0.086 mmol) was reacted to afford the title compound after prep. basic HPLC (19.2 mg, 57% yield). 1H NMR (400 MHz, DMSO-d6) δ 13.69 (s, 1H), 12.30 (s, 1H), 10.29 (s, 1H), 8.60-7.71 (m, 1H), 7.74-7.51 (m, 2H), 7.22 (d, J=8.5 Hz, 2H), 4.13 (s, 1H), 2.17 (s, 6H), 1.25-1.10 (m, 1H), 0.84-0.70 (m, 1H), 0.63 (s, 1H), 0.50-0.38 (m, 1H), 0.37-0.12 (m, 5H), 0.08-0.01 (m, 1H), −0.16 (s, 1H); LCMS (ES): m/z 391.224 [M+H]⁺, RT=1.99 min.

### Example 5: 3,3-dicyclopropyl-N-[4-(3,5-dimethyl-1H-pyrazol-4-yl)phenyl]-2-[5-(2-fluorophenyl)-4H-1,2,4-triazol-3-yl]propanamide

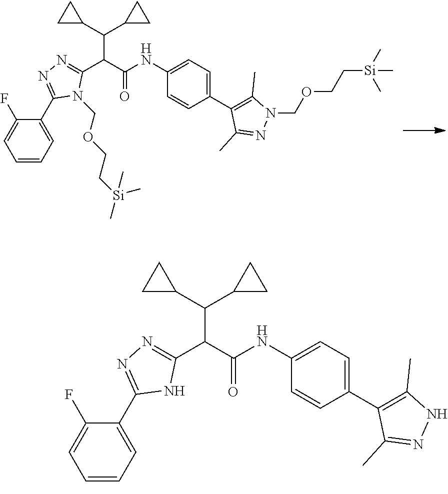

According to the method of Example 1 the compound of Preparation 20 (12 mg, 0.016 mmol) was reacted to afford the title compound after prep. acidic HPLC (5.7 mg, 73% yield). 1H NMR (400 MHz, DMSO-d6) δ 10.48 (s, 1H), 8.00 (td, J=7.7, 1.7 Hz, 1H), 7.73-7.64 (m, 2H), 7.53-7.43 (m, 1H), 7.37-7.24 (m, 4H), 4.24 (d, J=9.8 Hz, 1H), 2.24 (s, 6H), 1.23 (q, J=9.3 Hz, 1H), 0.90-0.76 (m, 1H), 0.76-0.61 (m, 1H), 0.53-0.02 (m, 7H), −0.01-−0.16 (m, 1H); LCMS (Method 3) (ES): m/z 485.3 [M+H]⁺, RT=0.72 min.

### Example 6: 3,3-dicyclopropyl-N-[4-(3,5-dimethyl-1H-pyrazol-4-yl)phenyl]-2-(1H-imidazol-2-yl)propanamide

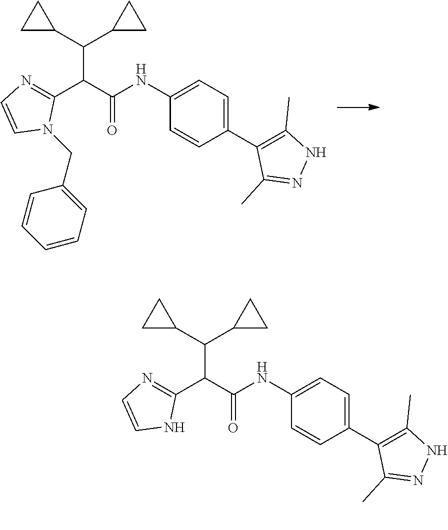

10% Pd/C (5 mg) was added to a nitrogen purged solution of the compound of Preparation 26 (24 mg, 0.05 mmol) in MeOH (2 mL) under balloon pressure of hydrogen and stirred at room temperature for 18 hours. The reaction mixture was diluted with MeOH (4 mL), filtered through Celite™ and concentrated in vacuo to give the title compound (5.7 mg, 29% yield). 1H NMR (400 MHz, DMSO-d6) δ 12.25 (s, 2H), 10.48 (s, 1H), 7.74-7.55 (m, 2H), 7.27-7.18 (m, 2H), 7.10 (s, 2H), 4.07 (d, 3=9.4 Hz, 1H), 2.17 (s, 6H), 1.12-0.99 (m, 1H), 0.81-0.67 (m, 1H), 0.65-0.53 (m, 1H), 0.48-0.39 (m, 1H), 0.38-0.26 (m, 2H), 0.25-0.10 (m, 3H), 0.07-0.01 (m, 1H), −0.21-−0.35 (m, 1H); LCMS (ES): m/z 390.229 [M+H]⁺, RT=1.85 min.

### Example 7: 3,3-dicyclopropyl-N-[4-(3,5-dimethyl-1H-pyrazol-4-yl)phenyl]-2-(5-methyl-4-phenyl-1H-imidazol-2-yl)propanamide

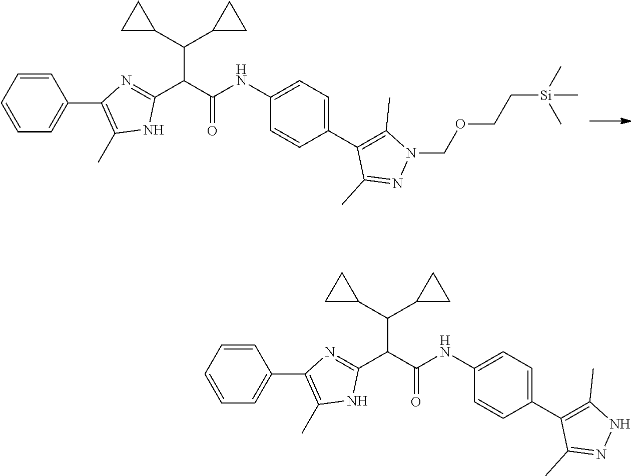 [SMILES: Cc1nn(COCC[Si](C)(C)C)c(C)c1-c1ccc(NC(=O)C(c2nc(-c3ccccc3)c(C)[nH]2)C(C2CC2)C2CC2)cc1 ; Cc1n[nH]c(C)c1-c1ccc(NC(=O)C(c2nc(-c3ccccc3)c(C)[nH]2)C(C2CC2)C2CC2)cc1]

According to the method of Example 1 the compound of Preparation 30 (25 mg, 0.042 mmol) was reacted to afford the title compound after prep. basic HPLC (14 mg, 69% yield). 1H NMR (600 MHz, DMSO-d6) δ 12.21 (s, 1H), 11.67 (s, 1H), 10.49 (s, 1H), 7.69-7.61 (m, 4H), 7.38-7.32 (m, 2H), 7.26-7.19 (m, 2H), 7.18-7.13 (m, 1H), 3.97 (d,J=9.7 Hz, 1H), 2.40 (s, 3H), 2.17 (s, 6H), 1.14 (q, J=9.3 Hz, 1H), 0.73 (dd, J=9.0, 4.5 Hz, 1H), 0.63 (dtd, J=13.7, 8.3, 5.1 Hz, 1H), 0.43 (td, J=10.1, 8.4, 5.5 Hz, 1H), 0.31 (qq, J=7.9, 4.0 Hz, 2H), 0.27-0.13 (m, 3H), 0.13-0.04 (m, 1H), −0.06 (d, J=4.9 Hz, 1H); LCMS (ES): m/z 480.277 [M+H]⁺, RT=2.08 min.

### Example 72: 3,3-dicyclopropyl-N-[4-(3,5-dimethyl-1H-pyrazol-4-yl)phenyl]-2-(4-fluoro-5-phenyl-1H-imidazol-2-yl)propanamide

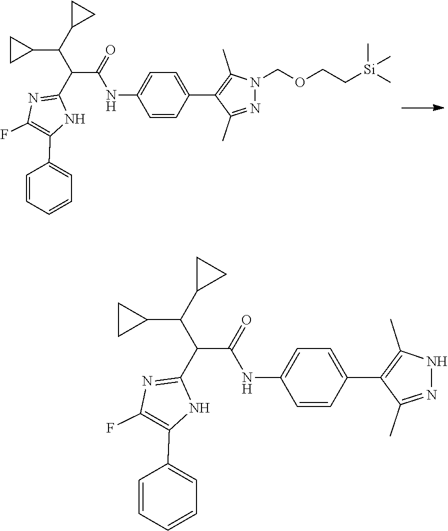 [SMILES: Cc1nn(COCC[Si](C)(C)C)c(C)c1-c1ccc(NC(=O)C(c2nc(F)c(-c3ccccc3)[nH]2)C(C2CC2)C2CC2)cc1 ; Cc1n[nH]c(C)c1-c1ccc(NC(=O)C(c2nc(F)c(-c3ccccc3)[nH]2)C(C2CC2)C2CC2)cc1]

According to the method of Example 1 the compound of Preparation 34 (1 mg, 0.0016 mmol) was reacted for 3 hours at room temperature to afford the title compound after prep. basic HPLC (0.5 mg, 60% yield). 1H NMR (600 MHz, DMSO-d6) δ 12.19 (d, J=37.6 Hz, 2H), 10.24 (s, 1H), 7.65-7.62 (m, 4H), 7.42 (t, J=7.6 Hz, 2H), 7.22 (d, J=8.6 Hz, 3H), 3.90 (d, J=10.3 Hz, 1H), 2.17 (br s, 6H), 1.37-1.28 (m, 1H), 0.82-0.61 (m, 2H), 0.48-0.07 (m, 7H), −0.019-−0.68 (m, 1H); LCMS (ES): m/z 484.251 [M+H]⁺, RT=2.50 min.

### Example 9: 3,3-dicyclopropyl-N-[4-(3,5-dimethyl-1H-pyrazol-4-yl)phenyl]-2-[5-(2-methoxyphenyl)-1H-imidazol-2-yl]propanamide

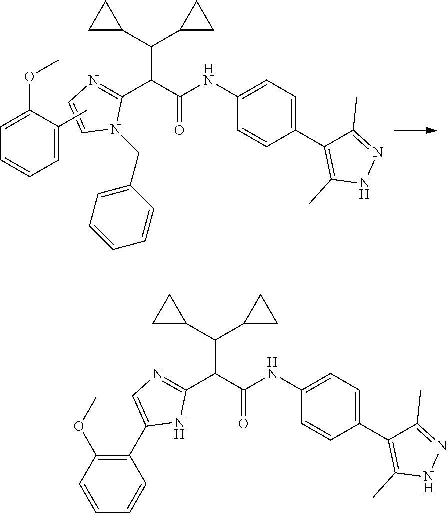

According to the method of Example 6 the compound of Preparation 39 (15 mg, 0.025 mmol) was reacted for 2 hours at room temperature. The reaction mixture was filtered through a PTFE filter and purified by prep. basic HPLC to afford the desired compound (6.1 mg, 49% yield). 1H NMR (600 MHz, DMSO-d6) δ 12.21 (s, 1H), 11.79 (s, 1H), 10.50 (s, 1H), 8.09 (dd, J=7.7, 1.8 Hz, 1H), 7.65 (d, J=8.6 Hz, 2H), 7.46 (s, 1H), 7.22 (d, J=8.6 Hz, 2H), 7.16 (ddd, J=8.7, 7.2, 1.8 Hz, 1H), 7.05-6.92 (m, 2H), 4.06 (d, J=9.5 Hz, 1H), 3.89 (s, 3H), 2.17 (s, 6H), 1.11 (q, J=9.2 Hz, 1H), 0.82-0.56 (m, 2H), 0.48-0.01 (m, 7H), −0.13 (sextet, J=4.9 Hz, 1H); LCMS (ES): m/z 496.271 [M+H]⁺, RT=2.11 min.

### Example 10: 2-(5-cyclopentyl-4H-1,2,4-triazol-3-yl)-3,3-dicyclopropyl-N-[4-(3,5-dimethyl-1H-pyrazol-4-yl)phenyl]propanamide

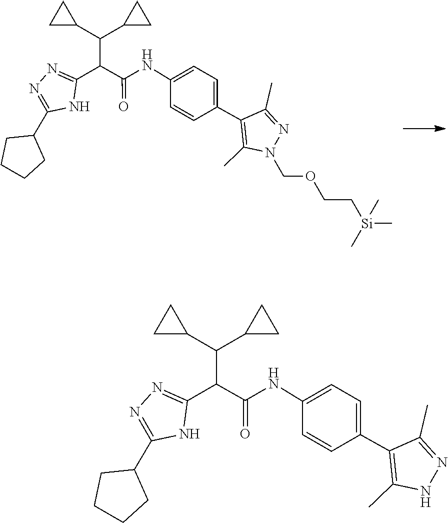 [SMILES: Cc1nn(COCC[Si](C)(C)C)c(C)c1-c1ccc(NC(=O)C(c2nnc(C3CCCC3)[nH]2)C(C2CC2)C2CC2)cc1 ; Cc1n[nH]c(C)c1-c1ccc(NC(=O)C(c2nnc(C3CCCC3)[nH]2)C(C2CC2)C2CC2)cc1]

According to the method of Example 1 the compound of Preparation 44 (24 mg, 0.041 mmol) was reacted for 1 hour at room temperature to afford the title compound after prep. basic HPLC (14 mg, 73% yield). 1H NMR (600 MHz, DMSO-d6) δ 13.26 (s, 1H), 12.22 (s, 1H), 10.24 (s, 1H), 7.61 (d, J=8.6 Hz, 2H), 7.21 (d, J=8.6 Hz, 2H), 3.98 (s, 1H), 3.12 (quintet, J=7.8 Hz, 1H), 2.17 (s, 6H), 1.96 (br s, 2H), 1.84-1.52 (m, 6H), 1.12 (q,J=9.2 Hz, 1H), 0.79-0.58 (m, 2H), 0.47-0.01 (m, 7H), −0.15 (br s, 1H); LCMS (ES): m/z 459.287 [M+H]⁺, RT=2.25 min.

### Example 11: 3,3-dicyclopropyl-N-[5-(3,5-dimethyl-1H-pyrazol-4-yl)-6-fluoro-2-pyridyl]-2-[5-(2-isopropylpyrazol-3-yl)-4H-1,2,4-triazol-3-yl]propanamide

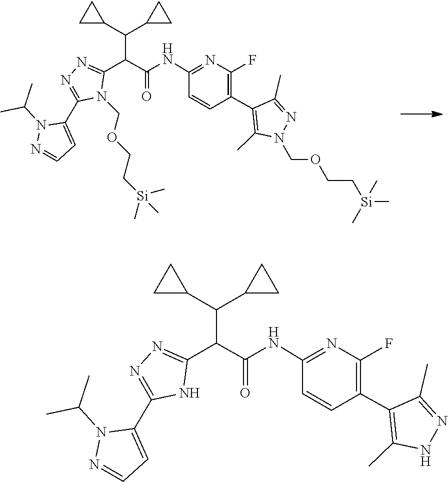

According to the method of Example 1 the compound of Preparation 52 (24 mg, 0.041 mmol) was reacted for 40 minutes at 40° C. to afford the title compound after prep. acidic HPLC (9.0 mg, 71% yield). 1H NMR (400 MHz, DMSO-d6) δ 14.09 (s, 1H), 11.09 (s, 1H), 8.19 (s, 1H), 8.05 (dd, J=8.1, 1.8 Hz, 1H), 7.88 (dd, J=10.1, 8.1 Hz, 1H), 7.51 (d, J=1.9 Hz, 1H), 6.66 (d, J=1.9 Hz, 1H), 5.57 (septet, J=6.6 Hz, 1H), 4.40 (d, J=9.3 Hz, 1H), 2.09 (s, 6H), 1.41 (dd, J=6.6, 4.5 Hz, 6H), 1.14 (q, J=9.3 Hz, 1H), 0.88-0.67 (m, 2H), 0.51-0.03 (m, 7H), −0.15 (dq, J=9.8, 5.1 Hz, 1H); LCMS (ES): m/z 518.279 [M+H]⁺, RT=2.31 min.

### Example 12: 3,3-dicyclopropyl-N-[4-(3,5-dimethyl-1H-pyrazol-4-yl)-2-fluoro-phenyl]-2-[5-(2-isopropylpyrazol-3-yl)-4H-1,2,4-triazol-3-yl]propanamide

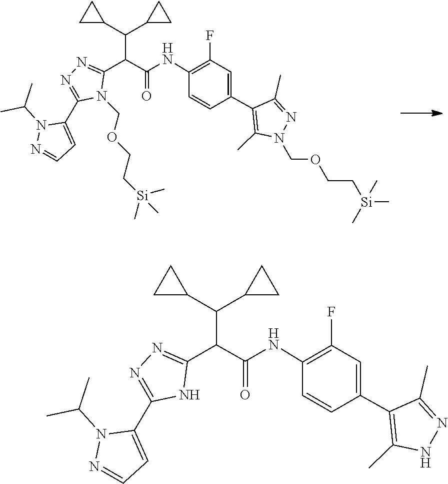

According to the method of Example 1 the compound of Preparation 54 (16 mg, 0.021 mmol) was reacted for 1 hour at 40° C. to afford the title compound after prep. acidic HPLC (9.0 mg, 85% yield). 1H NMR (400 MHz, DMSO-d6) δ 13.95 (s, 1H), 10.25 (s, 1H), 8.20 (s, 1H), 7.94 (t, J=8.5 Hz, 1H), 7.52 (d, J=1.8 Hz, 1H), 7.18 (dd, J=12.2, 1.9 Hz, 1H), 7.10 (dd, J=8.4, 1.9 Hz, 1H), 6.67 (d, J=1.9 Hz, 1H), 5.59 (septet, J=6.5 Hz, 1H), 4.41 (d, J=9.2 Hz, 1H), 2.20 (s, 6H), 1.41 (dd, J=6.6, 2.2 Hz, 6H), 1.13 (q, J=9.3 Hz, 1H), 0.87-0.66 (m, 2H), 0.53-0.04 (m, 7H), −0.14 (dq, J=9.8, 5.0 Hz, 1H); LCMS (ES): m/z 517.284 [M+H]⁺, RT=2.32 min.

### Example 13: 3,3-dicyclopropyl-N-[4-(3,5-dimethyl-1H-pyrazol-4-yl)phenyl]-2-[5-(4-methyl-1,2,5-oxadiazol-3-yl)-4H-1,2,4-triazol-3-yl]propanamide

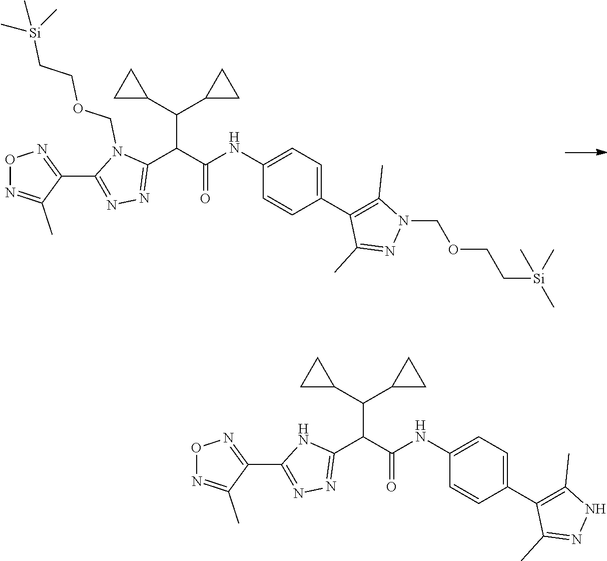

According to the method of Example 1 the compound of Preparation 62 (16 mg, 0.021 mmol) was reacted for 2 hours at 40° C. to afford the title compound after prep. acidic HPLC (9.0 mg, 85% yield). 1H NMR (400 MHz, DMSO-d6) δ 14.25 (s, 1H), 12.01 (s, 1H), 10.22 (s, 1H), 7.38 (d, J=8.6 Hz, 2H), 6.98 (d, J=8.6 Hz, 2H), 4.06 (d, J=10.0 Hz, 1H), 2.39 (s, 3H), 1.92 (s, 6H), 0.97 (q, J=9.4 Hz, 1H), 0.65-0.50 (m, 1H), 0.46-0.35 (m, 1H), 0.28-−0.24 (m, 7H), −0.40 (dq, J=9.8, 5.0 Hz, 1H); LCMS (ES): m/z 473.242 [M+H]⁺, RT=2.27 min.

### Example 14: 3,3-dicyclopropyl-N-[4-(3,5-dimethyl-1H-pyrazol-4-yl)phenyl]-2-[5-(4-fluoro-2-methyl-pyrazol-3-yl)-4H-1,2,4-triazol-3-yl]propanamide

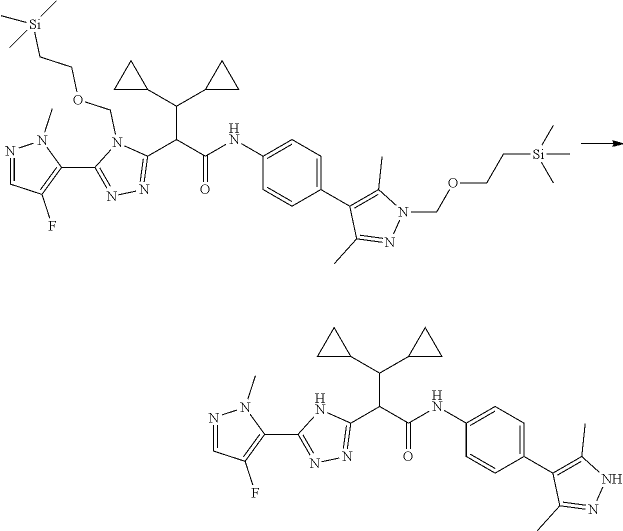

According to the method of Example 1 the compound of Preparation 70 (36 mg, 0.048 mmol) was reacted for 1.5 hours at 40° C. to afford the title compound after prep. acidic HPLC (19 mg, 83% yield). 1H NMR (400 MHz, DMSO-d6) δ 14.19 (s, 1H), 12.27 (s, 1H), 10.45 (s, 1H), 7.64 (d, J=8.6 Hz, 2H), 7.57 (d, J=4.3 Hz, 1H), 7.24 (d, J=8.6 Hz, 2H), 4.29 (d, J=10.0 Hz, 1H), 4.05 (s, 3H), 2.17 (s, 6H), 1.19 (q, J=9.7 Hz, 1H), 0.88-0.76 (m, 1H), 0.70-0.57 (m, 1H), 0.51-0.02 (m, 7H), −0.11-−0.23 (m, 1H); LCMS (ES): m/z 489.253 [M+H]⁺, RT=2.17 min.

### Example 15: 3,3-dicyclopropyl-N-[4-(3,5-dimethyl-1H-pyrazol-4-yl)phenyl]-2-[5-(3-pyridylmethyl)-4H-1,2,4-triazol-3-yl]propanamide

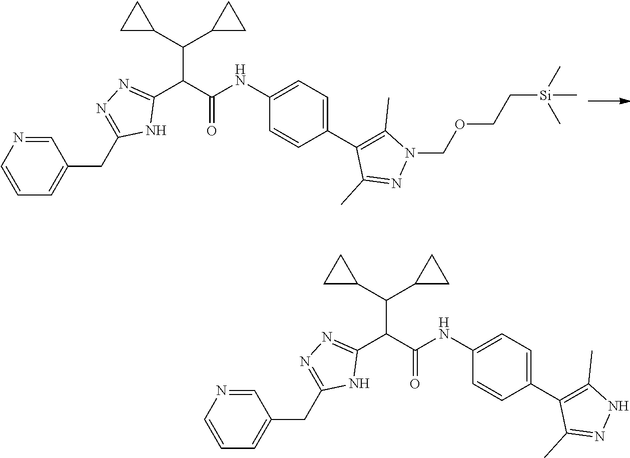 [SMILES: Cc1nn(COCC[Si](C)(C)C)c(C)c1-c1ccc(NC(=O)C(c2nnc(Cc3cccnc3)[nH]2)C(C2CC2)C2CC2)cc1 ; Cc1n[nH]c(C)c1-c1ccc(NC(=O)C(c2nnc(Cc3cccnc3)[nH]2)C(C2CC2)C2CC2)cc1]

According to the method of Example 1 the compound of Preparation 78 (68 mg, 0.11 mmol) was reacted for 40 minutes at 40° C. to afford the title compound after prep. basic HPLC (32 mg, 52% yield). 1H NMR (400 MHz, DMSO-d6) δ 13.46 (s, 1H), 10.29 (s, 1H), 8.51 (d, J=2.3 Hz, 1H), 8.42 (d, J=4.6 Hz, 1H), 7.67 (dt, J=8.0, 2.0 Hz, 1H), 7.59 (d, J=8.6 Hz, 2H), 7.32 (dd, J=7.8, 4.8 Hz, 1H), 7.21 (d, J=8.6 Hz, 2H), 4.03 (br s, 3H), 2.17 (s, 6H), 1.10 (q, J=9.4 Hz, 1H), 0.82-0.71 (m, 1H), 0.60 (br s, 1H), 0.47-0.07 (m, 6H), 0.02-−0.07 (m, 1H), −0.21 (br s, 1H); LCMS (ES): m/z 482.267 [M+H]⁺, RT=1.88 min.

### Example 16: 3,3-dicyclopropyl-N-[4-(3,5-dimethyl-1H-pyrazol-4-yl)phenyl]-2-[5-(1H-pyrazol-3-ylmethyl)-4H-1,2,4-triazol-3-yl]propanamide

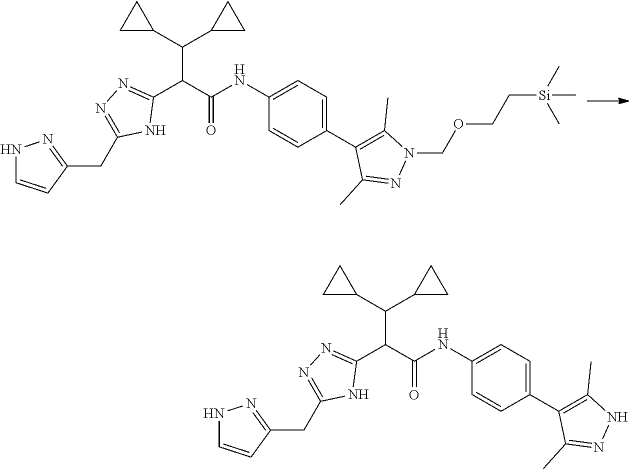 [SMILES: Cc1nn(COCC[Si](C)(C)C)c(C)c1-c1ccc(NC(=O)C(c2nnc(Cc3cc[nH]n3)[nH]2)C(C2CC2)C2CC2)cc1 ; Cc1n[nH]c(C)c1-c1ccc(NC(=O)C(c2nnc(Cc3cc[nH]n3)[nH]2)C(C2CC2)C2CC2)cc1]

According to the method of Example 1 the compound of Preparation 80 (18 mg, 0.030 mmol) was reacted for 90 minutes at room temperature to afford the title compound after prep. basic HPLC (3.8 mg, 27% yield). 1H NMR (400 MHz, DMSO-d6) δ 13.77-12.00 (m, 3H), 10.25 (s, 1H), 7.67-7.42 (m, 3H), 7.21 (d, J=8.6 Hz, 2H), 6.03 (d, J=2.0 Hz, 1H), 4.05-3.91 (m, 3H), 2.17 (s, 6H), 1.12 (q, J=9.3 Hz, 1H), 0.81-0.58 (m, 2H), 0.47-0.10 (m, 6H), 0.07-0.00 (m, 1H), −0.09-−0.21 (m, 1H); LCMS (ES): m/z 471.261 [M+H]⁺, RT=1.97 min.

### Example 17: 2-(5-benzyl-4H-1,2,4-triazol-3-yl)-3,3-dicyclopropyl-N-[4-(3,5-dimethyl-1H-pyrazol-4-yl)phenyl]propanamide

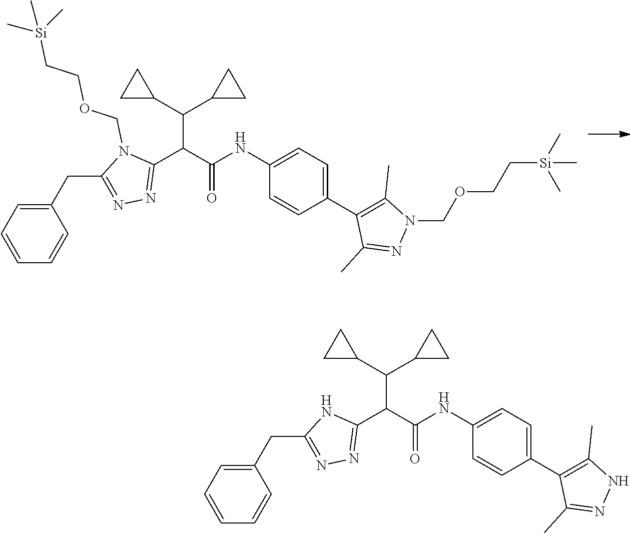 [SMILES: Cc1nn(COCC[Si](C)(C)C)c(C)c1-c1ccc(NC(=O)C(c2nnc(Cc3ccccc3)n2COCC[Si](C)(C)C)C(C2CC2)C2CC2)cc1 ; Cc1n[nH]c(C)c1-c1ccc(NC(=O)C(c2nnc(Cc3ccccc3)[nH]2)C(C2CC2)C2CC2)cc1]

According to the method of Example 1 the compound of Preparation 76 (18 mg, 0.030 mmol) was reacted for 1 hour at 40° C. to afford the title compound after prep. acidic HPLC (30 mg, 100% yield). 1H NMR (400 MHz, DMSO-d6) δ 14.02-11.02 (m, 2H), 10.25 (s, 1H), 7.59 (d, J=8.6 Hz, 2H), 7.33-7.12 (m, 7H), 3.99 (br s, 3H), 2.17 (s, 6H), 1.11 (q, J=9.2 Hz, 1H), 0.82-0.53 (m, 2H), 0.46-0.08 (m, 6H), 0.04-−0.05 (m, 1H), −0.18 (br s, 1H); LCMS (ES): m/z 481.272 [M+H]⁺, RT=2.24 min.

### Example 18: 2-[5-(2-chlorophenyl)-4H-1,2,4-triazol-3-yl]-3,3-dicyclopropyl-N-[4-(3,5-dimethyl-1H-pyrazol-4-yl)phenyl]propanamide

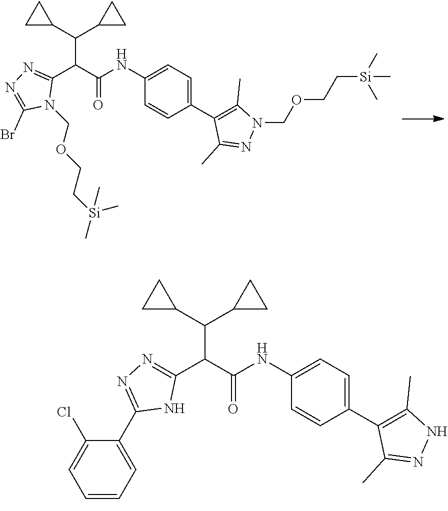

(2-Chlorophenyl)boronic acid (14.9 mg, 0.095 mmol) was added to the crude compound of Preparation 19 (23.1 mg, 0.032 mmol) and the reaction mixture was degassed with nitrogen for 10 minutes. Pd(dppf)Cl₂·DCM (2.7 mg, 0.003 mmol) was added and the reaction mixture was stirred at 90° C. for 30 minutes. The cooled reaction mixture was filtered through a PTFE filter and purified directly by prep. acidic HPLC to afford 2-[5-(2-chlorophenyl)-4-(2-trimethylsilylethoxymethyl)-1,2,4-triazol-3-yl]-3,3-dicyclopropyl-N-[4-[3,5-dimethyl-1-(2-trimethylsilylethoxymethyl)pyrazol-4-yl]phenyl]propanamide (11 mg, 45.6% yield). TFA (1 mL) was added to a solution of 2-[5-(2-chlorophenyl)-4-(2-trimethylsilylethoxymethyl)-1,2,4-triazol-3-yl]-3,3-dicyclopropyl-N-[4-[3,5-dimethyl-1-(2-trimethylsilylethoxymethyl)pyrazol-4-yl]phenyl]propanamide (11 mg, 0.0145 mmol) in DCM (1 mL) at room temperature. After 1 hour the reaction mixture was concentrated in vacuo, redissolved in MeOH (2 mL) and purified by prep. acidic HPLC to afford the title compound (5.0 mg, 69% yield). 1H NMR (400 MHz, DMSO) δ 14.01 (s, 1H), 12.23 (s, 1H), 10.43 (s, 1H), 7.87-7.77 (m, 1H), 7.64 (d, J=8.3 Hz, 2H), 7.59-7.53 (m, 1H), 7.44 (dd, J=6.3, 3.0 Hz, 2H), 7.23 (d, J=8.2 Hz, 2H), 4.22 (d, J=9.6 Hz, 1H), 2.17 (s, 6H), 1.34-1.11 (m, 1H), 0.95-0.75 (m, 1H), 0.74-0.60 (m, 1H), 0.50-0.02 (m, 7H), −0.06-−0.17 (m, 1H). LCMS (ES): m/z 501.217 [M+H]⁺, RT=2.34 min.

General Method A: According to the method of Preparation 20, the appropriate commercially available boronic acid or ester (2-3 equivalents) was added to a mixture of the bromotriazole of Preparation 19 in DMF (0.5-1.0 mL) and potassium carbonate (200 mg/mL in water, 3-5 equivalents) and the reaction mixture was degassed with nitrogen for 10 minutes. Pd(dppf)Cl₂·DCM (10 mol %) was added and the reaction mixture was stirred at 90° C. for 30 minutes. The cooled reaction mixture was filtered through a PTFE filter and purified directly by prep. acidic HPLC to afford the desired intermediate compound. TFA (1 mL) was added to a solution of the intermediate compound in DCM (1 mL) and stirred at a temperature from room temperature, up to 45° C., for 1 hour. The reaction mixture was concentrated in vacuo, dissolved in MeOH and purified directly by prep. acidic HPLC to afford the desired title compound.

The examples listed in the table below were all accessed using General Method A.

| Ex. | | | LCMS | Mass |
|---|---|---|---|---|
| No. | Structure | Name | RT | ion |
| 19 | 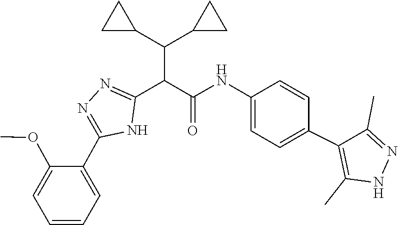 [SMILES: COc1ccccc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(2-methoxyphenyl)-4H-1,2,4- triazol-3-yl]propanamide | 2.35 | 497.267 |
| 20 | 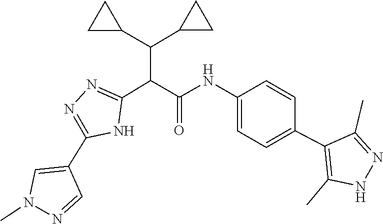 [SMILES: Cc1n[nH]c(C)c1-c1ccc(NC(=O)C(c2nnc(-c3cnn(C)c3)[nH]2)C(C2CC2)C2CC2)cc1] | 3,3-dicyclopropyl-N-[4-(3,5- dimethyl-1H-pyrazol-4-yl)phenyl]-2- [5-(1-methylpyrazol-4-yl)-4H-1,2,4- triazol-3-yl]propanamide | 2.03 | 471.262 |
| 21 | 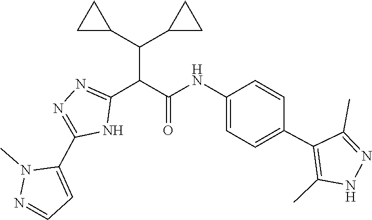 [SMILES: Cc1n[nH]c(C)c1-c1ccc(NC(=O)C(c2nnc(-c3ccnn3C)[nH]2)C(C2CC2)C2CC2)cc1] | 3,3-dicyclopropyl-N-[4-(3,5- dimethyl-1H-pyrazol-4-yl)phenyl]-2- [5-(2-methylpyrazol-3-yl)-4H-1,2,4- triazol-3-yl]propanamide | 2.12 | 471.262 |
| 22 | 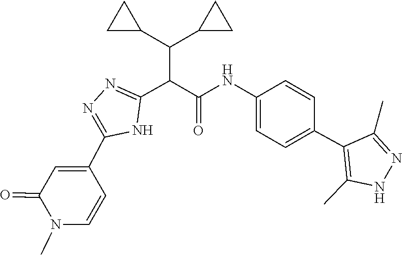 [SMILES: Cc1n[nH]c(C)c1-c1ccc(NC(=O)C(c2nnc(-c3ccn(C)c(=O)c3)[nH]2)C(C2CC2)C2CC2)cc1] | 3,3-dicyclopropyl-N-[4-(3,5- dimethyl-1H-pyrazol-4-yl)phenyl]-2- [5-(1-methyl-2-oxo-4-pyridyl)-4H- 1,2,4-triazol-3-yl]propanamide | 2.01 | 498.261 |
| 23 | 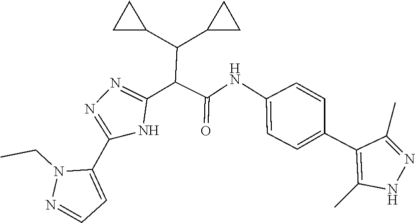 [SMILES: CCn1nccc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(2-ethylpyrazol-3-yl)-4H-1,2,4- triazol-3-yl]propanamide | 2.18 | 485.278 |
| 24 | 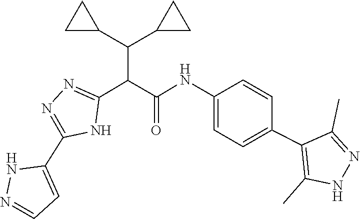 [SMILES: Cc1n[nH]c(C)c1-c1ccc(NC(=O)C(c2nnc(-c3ccn[nH]3)[nH]2)C(C2CC2)C2CC2)cc1] | 3,3-dicyclopropyl-N-[4-(3,5- dimethyl-1H-pyrazol-4-yl)phenyl]-2- [5-(1H-pyrazol-5-yl)-4H-1,2,4- triazol-3-yl]propanamide | 2.01 | 457.246 |
| 25 | 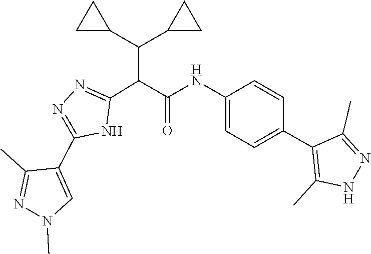 [SMILES: Cc1nn(C)cc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(1,3-dimethylpyrazol-4-yl)-4H- 1,2,4-triazol-3-yl]propanamide | 2.07 | 485.277 |
| 26 | 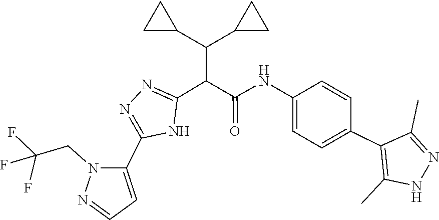 [SMILES: Cc1n[nH]c(C)c1-c1ccc(NC(=O)C(c2nnc(-c3ccnn3CC(F)(F)F)[nH]2)C(C2CC2)C2CC2)cc1] | 3,3-dicyclopropyl-N-[4-(3,5- dimethyl-1H-pyrazol-4-yl)phenyl]-2- [5-[2-(2,2,2-trifluoroethyl)pyrazol-3- yl]-4H-1,2,4-triazol-3- yl]propanamide | 2.19 | 539.250 |
| 27 | 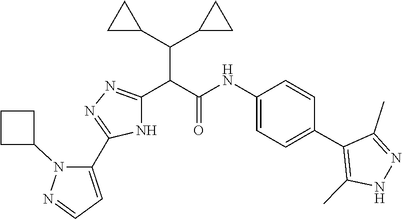 | 2-[5-(2-cyclobutylpyrazol-3-yl)-4H- 1,2,4-triazol-3-yl]-3,3-dicyclopropyl- N-[4-(3,5-dimethyl-1H-pyrazol-4- yl)phenyl]propanamide | 2.30 | 511.293 |
| 28 | 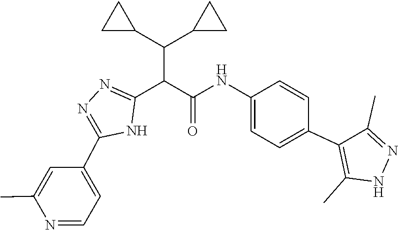 [SMILES: Cc1cc(-c2nnc(C(C(=O)Nc3ccc(-c4c(C)n[nH]c4C)cc3)C(C3CC3)C3CC3)[nH]2)ccn1] | 3,3-dicyclopropyl-N-[4-(3,5- dimethyl-1H-pyrazol-4-yl)phenyl]-2- [5-(2-methyl-4-pyridyl)-4H-1,2,4- triazol-3-yl]propanamide | 1.96 | 482.267 |
| 29 | 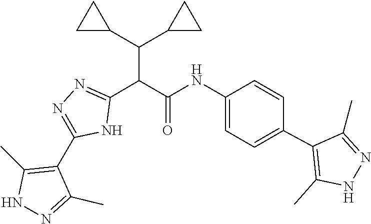 [SMILES: Cc1n[nH]c(C)c1-c1ccc(NC(=O)C(c2nnc(-c3c(C)n[nH]c3C)[nH]2)C(C2CC2)C2CC2)cc1] | 3,3-dicyclopropyl-N-[4-(3,5- dimethyl-1H-pyrazol-4-yl)phenyl]-2- [5-(3,5-dimethyl-1H-pyrazol-4-yl)- 4H-1,2,4-triazol-3-yl]propanamide | 2.04 | 485.277 |
| 30 | 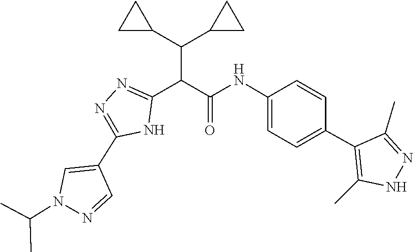 [SMILES: Cc1n[nH]c(C)c1-c1ccc(NC(=O)C(c2nnc(-c3cnn(C(C)C)c3)[nH]2)C(C2CC2)C2CC2)cc1] | 3,3-dicyclopropyl-N-[4-(3,5- dimethyl-1H-pyrazol-4-yl)phenyl]-2- [5-(1-isopropylpyrazol-4-yl)-4H- 1,2,4-triazol-3-yl]propanamide | 2.13 | 499.294 |
| 31 | 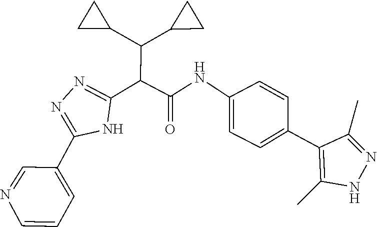 [SMILES: Cc1n[nH]c(C)c1-c1ccc(NC(=O)C(c2nnc(-c3cccnc3)[nH]2)C(C2CC2)C2CC2)cc1] | 3,3-dicyclopropyl-N-[4-(3,5- dimethyl-1H-pyrazol-4-yl)phenyl]-2- [5-(3-pyridyl)-4H-1,2,4-triazol-3- yl]propanamide | 2.06 | 468.251 |
| 32 | 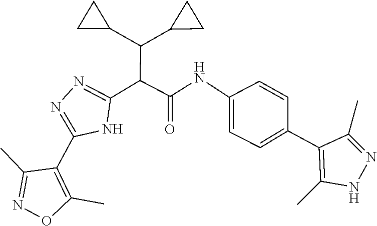 [SMILES: Cc1n[nH]c(C)c1-c1ccc(NC(=O)C(c2nnc(-c3c(C)noc3C)[nH]2)C(C2CC2)C2CC2)cc1] | 3,3-dicyclopropyl-2-[5-(3,5- dimethylisoxazol-4-yl)-4H-1,2,4- triazol-3-yl]-N-[4-(3,5-dimethyl-1H- pyrazol-4-yl)phenyl]propanamide | 2.25 | 486.262 |
| 33 | 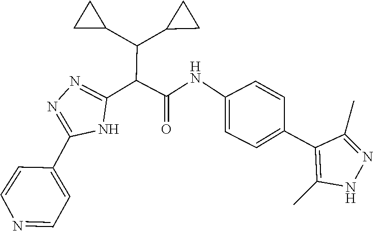 [SMILES: Cc1n[nH]c(C)c1-c1ccc(NC(=O)C(c2nnc(-c3ccncc3)[nH]2)C(C2CC2)C2CC2)cc1] | 3,3-dicyclopropyl-N-[4-(3,5- dimethyl-1H-pyrazol-4-yl)phenyl]-2- [5-(4-pyridyl)-4H-1,2,4-triazol-3- yl]propanamide | 2.00 | 468.251 |
| 34 | 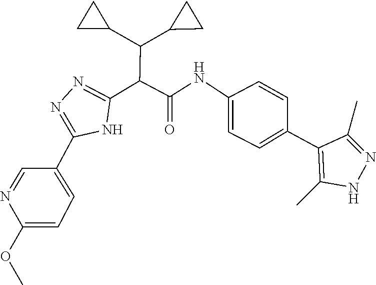 [SMILES: COc1ccc(-c2nnc(C(C(=O)Nc3ccc(-c4c(C)n[nH]c4C)cc3)C(C3CC3)C3CC3)[nH]2)cn1] | 3,3-dicyclopropyl-N-[4-(3,5- dimethyl-1H-pyrazol-4-yl)phenyl]-2- [5-(6-methoxy-3-pyridyl)-4H-1,2,4- triazol-3-yl]propanamide | 2.24 | 498.262 |
| 35 | 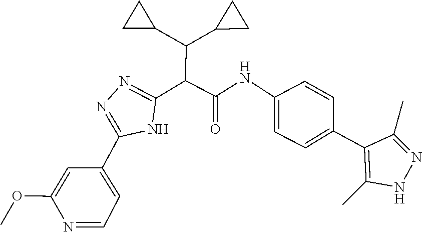 [SMILES: COc1cc(-c2nnc(C(C(=O)Nc3ccc(-c4c(C)n[nH]c4C)cc3)C(C3CC3)C3CC3)[nH]2)ccn1] | 3,3-dicyclopropyl-N-[4-(3,5- dimethyl-1H-pyrazol-4-yl)phenyl]-2- [5-(2-methoxy-4-pyridyl)-4H-1,2,4- triazol-3-yl]propanamide | 2.24 | 498.262 |
| 36 | 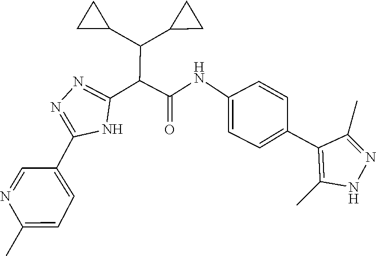 [SMILES: Cc1ccc(-c2nnc(C(C(=O)Nc3ccc(-c4c(C)n[nH]c4C)cc3)C(C3CC3)C3CC3)[nH]2)cn1] | 3,3-dicyclopropyl-N-[4-(3,5- dimethyl-1H-pyrazol-4-yl)phenyl]-2- [5-(6-methyl-3-pyridyl)-4H-1,2,4- triazol-3-yl]propanamide | 2.03 | 482.267 |
| 37 | 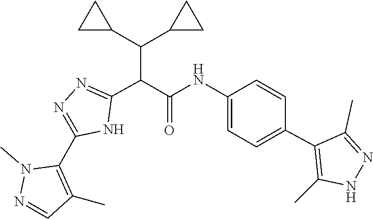 [SMILES: Cc1cnn(C)c1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(2,4-dimethylpyrazol-3-yl)-4H- 1,2,4-triazol-3-yl]propanamide | 2.17 | 485.277 |
| 38 | 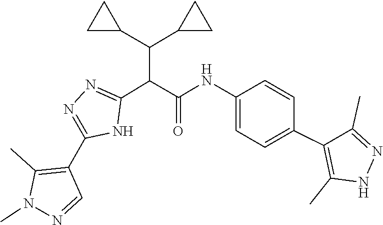 [SMILES: Cc1n[nH]c(C)c1-c1ccc(NC(=O)C(c2nnc(-c3cnn(C)c3C)[nH]2)C(C2CC2)C2CC2)cc1] | 3,3-dicyclopropyl-N-[4-(3,5- dimethyl-1H-pyrazol-4-yl)phenyl]-2- [5-(1,5-dimethylpyrazol-4-yl)-4H- 1,2,4-triazol-3-yl]propanamide | 2.08 | 485.278 |
| 39 | 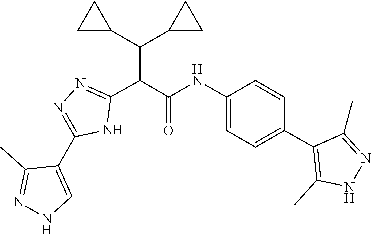 [SMILES: Cc1n[nH]cc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(3-methyl-1H-pyrazol-4-yl)-4H- 1,2,4-triazol-3-yl]propanamide | 2.02 | 471.262 |
| 40 | 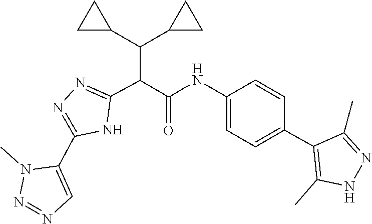 [SMILES: Cc1n[nH]c(C)c1-c1ccc(NC(=O)C(c2nnc(-c3cnnn3C)[nH]2)C(C2CC2)C2CC2)cc1] | 3,3-dicyclopropyl-N-[4-(3,5- dimethyl-1H-pyrazol-4-yl)phenyl]-2- [5-(3-methyltriazol-4-yl)-4H-1,2,4- triazol-3-yl]propanamide | 2.07 | 472.257 |
| 41 | 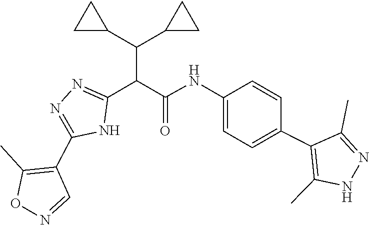 [SMILES: Cc1n[nH]c(C)c1-c1ccc(NC(=O)C(c2nnc(-c3cnoc3C)[nH]2)C(C2CC2)C2CC2)cc1] | 3,3-dicyclopropyl-N-[4-(3,5- dimethyl-1H-pyrazol-4-yl)phenyl]-2- [5-(5-methylisoxazol-4-yl)-4H- 1,2,4-triazol-3-yl]propanamide | 2.13 | 472.246 |
| 42 | 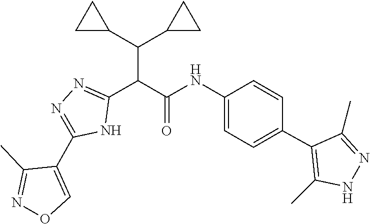 [SMILES: Cc1nocc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(3-methylisoxazol-4-yl)-4H- 1,2,4-triazol-3-yl]propanamide | 2.19 | 472.246 |

General Method B: According to the method of Preparation 20, the bromotriazole of Preparation 19 (0.041-0.62 mmol), the appropriate commercially available boronic acid or ester (2 equivalents), potassium phosphate (3 equivalents) and dioxane (1-5 mL) were mixed and degassed with nitrogen for 3 minutes. Palladium tetrakis (8 mol %) was added and the reaction mixture was stirred at 100° C. for 4-18 hours. The cooled reaction mixture was filtered through a PTFE filter and purified directly by prep. acidic HPLC to afford the desired SEM protected intermediate compounds. SEM-deprotection was carried out as for method A.

The examples listed in the table below were all accessed using General Method B.

| Ex. | | | LCMS | Mass |
|---|---|---|---|---|
| No. | Structure | Name | RT | ion |
| 43 | 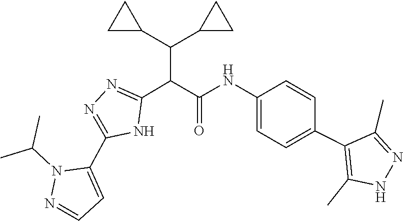 [SMILES: Cc1n[nH]c(C)c1-c1ccc(NC(=O)C(c2nnc(-c3ccnn3C(C)C)[nH]2)C(C2CC2)C2CC2)cc1] | 3,3-dicyclopropyl-N-[4-(3,5- dimethyl-1H-pyrazol-4-yl)phenyl]-2- [5-(2-isopropylpyrazol-3-yl)-4H- 1,2,4-triazol-3-yl]propanamide | 2.24 | 499.293 |
| 44 | 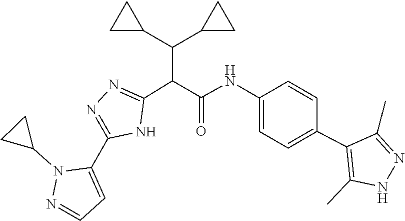 [SMILES: Cc1n[nH]c(C)c1-c1ccc(NC(=O)C(c2nnc(-c3ccnn3C3CC3)[nH]2)C(C2CC2)C2CC2)cc1] | 3,3-dicyclopropyl-2-[5-(2- cyclopropylpyrazol-3-yl)-4H-1,2,4- triazol-3-yl]-N-[4-(3,5-dimethyl-1H- pyrazol-4-yl)phenyl]propanamide | 2.18 | 497.278 |
| 45 | 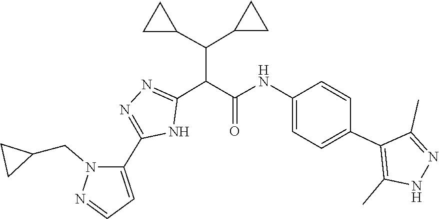 [SMILES: Cc1n[nH]c(C)c1-c1ccc(NC(=O)C(c2nnc(-c3ccnn3CC3CC3)[nH]2)C(C2CC2)C2CC2)cc1] | 3,3-dicyclopropyl-2-[5-[2- (cyclopropylmethyl)pyrazol-3-yl]- 4H-1,2,4-triazol-3-yl]-N-[4-(3,5- dimethyl-1H-pyrazol-4- yl)phenyl]propanamide | 2.23 | 511.294 |
| 46 | 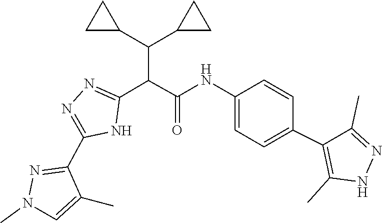 [SMILES: Cc1cn(C)nc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(1,4-dimethylpyrazol-3-yl)-4H- 1,2,4-triazol-3-yl]propanamide | 2.14 | 485.278 |
| 47 | 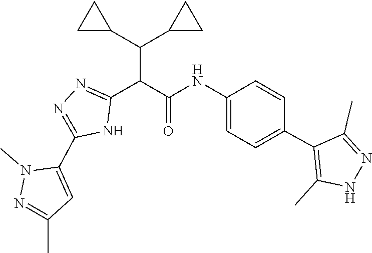 [SMILES: Cc1cc(-c2nnc(C(C(=O)Nc3ccc(-c4c(C)n[nH]c4C)cc3)C(C3CC3)C3CC3)[nH]2)n(C)n1] | 3,3-dicyclopropyl-N-[4-(3,5- dimethyl-1H-pyrazol-4-yl)phenyl]-2- [5-(2,5-dimethylpyrazol-3-yl)-4H- 1,2,4-triazol-3-yl]propanamide | 2.13 | 485.278 |
| 48 | 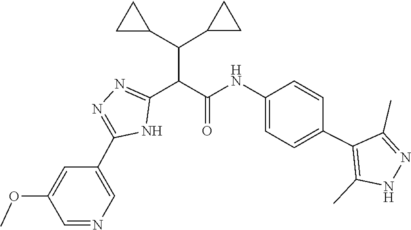 [SMILES: COc1cncc(-c2nnc(C(C(=O)Nc3ccc(-c4c(C)n[nH]c4C)cc3)C(C3CC3)C3CC3)[nH]2)c1] | 3,3-dicyclopropyl-N-[4-(3,5- dimethyl-1H-pyrazol-4-yl)phenyl]-2- [5-(5-methoxy-3-pyridyl)-4H-1,2,4- triazol-3-yl]propanamide | 2.11 | 498.262 |
| 49 | 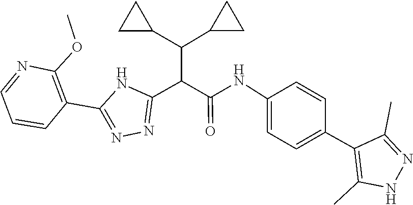 [SMILES: COc1ncccc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(2-methoxy-3-pyridyl)-4H-1,2,4- triazol-3-yl]propanamide | 2.24 | 498.262 |
| 50 | 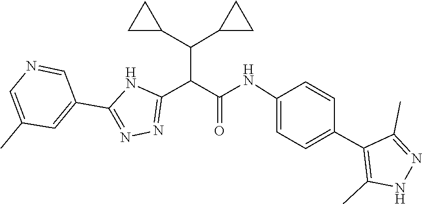 [SMILES: Cc1cncc(-c2nnc(C(C(=O)Nc3ccc(-c4c(C)n[nH]c4C)cc3)C(C3CC3)C3CC3)[nH]2)c1] | 3,3-dicyclopropyl-N-[4-(3,5- dimethyl-1H-pyrazol-4-yl)phenyl]-2- [5-(5-methyl-3-pyridyl)-4H-1,2,4- triazol-3-yl]propanamide | 2.06 | 482.267 |
| 51 | 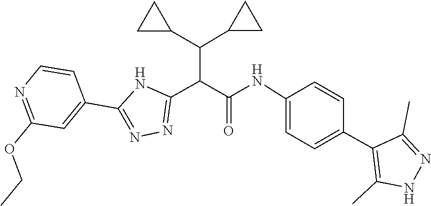 [SMILES: CCOc1cc(-c2nnc(C(C(=O)Nc3ccc(-c4c(C)n[nH]c4C)cc3)C(C3CC3)C3CC3)[nH]2)ccn1] | 3,3-dicyclopropyl-N-[4-(3,5- dimethyl-1H-pyrazol-4-yl)phenyl]-2- [5-(2-ethoxy-4-pyridyl)-4H-1,2,4- triazol-3-yl]propanamide | 2.30 | 512.278 |
| 52 | 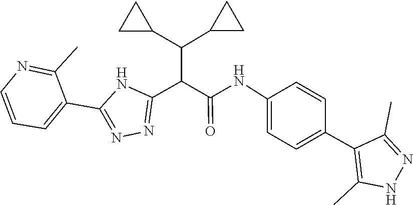 [SMILES: Cc1ncccc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(2-methyl-3-pyridyl)-4H-1,2,4- triazol-3-yl]propanamide | 1.95 | 482.267 |
| 53 | 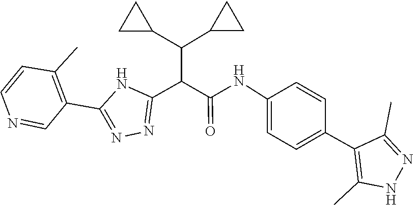 [SMILES: Cc1ccncc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(4-methyl-3-pyridyl)-4H-1,2,4- triazol-3-yl]propanamide | 2.00 | 482.267 |
| 54 | 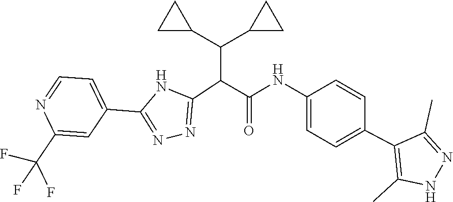 [SMILES: Cc1n[nH]c(C)c1-c1ccc(NC(=O)C(c2nnc(-c3ccnc(C(F)(F)F)c3)[nH]2)C(C2CC2)C2CC2)cc1] | 3,3-dicyclopropyl-N-[4-(3,5- dimethyl-1H-pyrazol-4-yl)phenyl]-2- [5-[2-(trifluoromethyl)-4-pyridyl]- 4H-1,2,4-triazol-3-yl]propanamide | 2.35 | 536.238 |
| 55 | 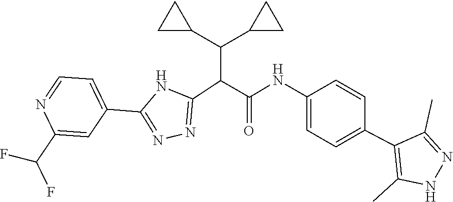 [SMILES: Cc1n[nH]c(C)c1-c1ccc(NC(=O)C(c2nnc(-c3ccnc(C(F)F)c3)[nH]2)C(C2CC2)C2CC2)cc1] | 3,3-dicyclopropyl-2-[5-[2- (difluoromethyl)-4-pyridyl]-4H- 1,2,4-triazol-3-yl]-N-[4-(3,5- dimethyl-1H-pyrazol-4- yl)phenyl] propanamide | 2.23 | 518.248 |
| 56 | 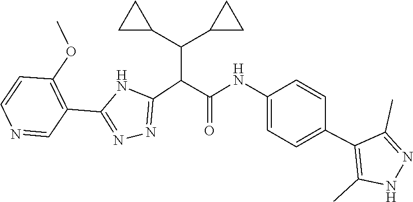 [SMILES: COc1ccncc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(4-methoxy-3-pyridyl)-4H-1,2,4- triazol-3-yl]propanamide | 1.90 | 498.262 |
| 57* | 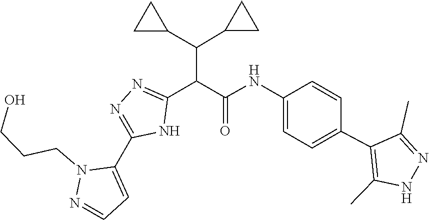 [SMILES: Cc1n[nH]c(C)c1-c1ccc(NC(=O)C(c2nnc(-c3ccnn3CCCO)[nH]2)C(C2CC2)C2CC2)cc1] | 3,3-dicyclopropyl-N-[4-(3,5- dimethyl-1H-pyrazol-4-yl)phenyl]-2- [5-[2-(3-hydroxypropyl)pyrazol-3- yl]-4H-1,2,4-triazol-3- yl]propanamide | 2.04 | 515.288 |

*-compound not purified by HPLC after Suzuki reaction. The crude intermediate was extracted from the reaction mixture with Et₂O, dried over MgSO₄, filtered and concentrated in vacuo to give the intermediate compound.

General Method C: According to the method of Preparation 20, the bromotriazole of Preparation 19 (0.041 mmol), the appropriate commercially available boronic acid or ester (2-3 equivalents), cesium carbonate (2-4 equivalents), dppf (10-50 mol %), palladium diacetate (5-25 mol %) and copper chloride (1 equivalent) were dissolved in DMF (1 mL) and degassed with nitrogen for 10 minutes. The reaction mixture was stirred at 100° C. for 18 hours. The cooled reaction mixture was filtered through a PTFE filter and purified directly by prep. acidic HPLC to afford the desired intermediate SEM protected compounds. SEM-deprotection was carried out as for method A.

The examples listed in the table below were all accessed using General Method C.

| Ex. | | | LCMS | Mass |
|---|---|---|---|---|
| No. | Structure | Name | RT | ion |
| 58 | 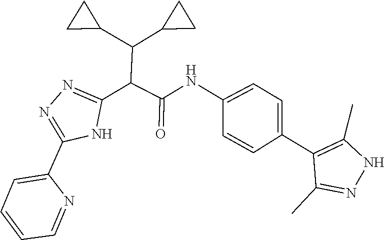 [SMILES: Cc1n[nH]c(C)c1-c1ccc(NC(=O)C(c2nnc(-c3ccccn3)[nH]2)C(C2CC2)C2CC2)cc1] | 3,3-dicyclopropyl-N-[4-(3,5- dimethyl-1H-pyrazol-4-yl)phenyl]-2- [5-(2-pyridyl)-4H-1,2,4-triazol-3- yl]propanamide | 2.09 | 468.251 |
| 59 | 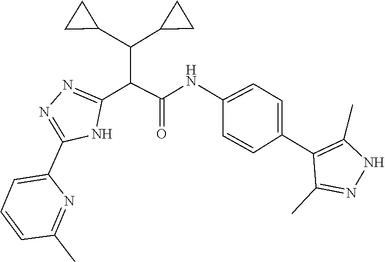 [SMILES: Cc1cccc(-c2nnc(C(C(=O)Nc3ccc(-c4c(C)n[nH]c4C)cc3)C(C3CC3)C3CC3)[nH]2)n1] | 3,3-dicyclopropyl-N-[4-(3,5- dimethyl-1H-pyrazol-4-yl)phenyl]-2- [5-(6-methyl-2-pyridyl)-4H-1,2,4- triazol-3-yl]propanamide | 2.13 | 482.266 |
| 60 | 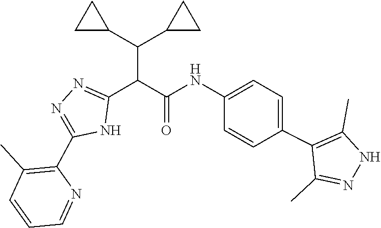 [SMILES: Cc1cccnc1-c1nnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(3-methyl-2-pyridyl)-4H-1,2,4- triazol-3-yl]propanamide | 2.14 | 482.267 |

Method D: Suzuki coupling conditions as for method A, reacting the bromotriazole of Preparation 19 with the appropriate commercially available vinylboronic acid or ester to give an intermediate alkene which was dissolved in MeOH. Pd/C was added under a nitrogen atmosphere, which was then exchanged for hydrogen. The mixture was stirred for 1 hour under balloon pressure. The reaction mixture was then filtered through a PTFE filter and evaporated to give a SEM protected intermediate. SEM-deprotection was carried out as for method A.

The examples listed in the table below were all accessed using General Method D.

| | Precursor | | | | |
|---|---|---|---|---|---|
| Ex. | Prep. | | | LCMS | Mass |
| No. | number | Structure | Name | RT | ion |
| 61 | 19 | 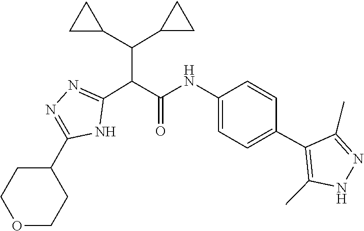 [SMILES: Cc1n[nH]c(C)c1-c1ccc(NC(=O)C(c2nnc(C3CCOCC3)[nH]2)C(C2CC2)C2CC2)cc1] | 3,3-dicyclopropyl-N-[4- (3,5-dimethyl-1H- pyrazol-4-yl)phenyl]-2- (5-tetrahydropyran-4-yl- 4H-1,2,4-triazol-3- yl)propanamide | 2.05 | 475.282 |
| 62 | 19 | 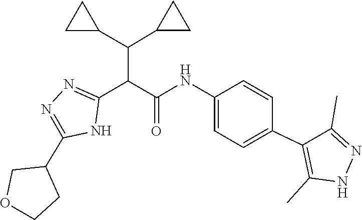 [SMILES: Cc1n[nH]c(C)c1-c1ccc(NC(=O)C(c2nnc(C3CCOC3)[nH]2)C(C2CC2)C2CC2)cc1] | 3,3-dicyclopropyl-N-[4- (3,5-dimethyl-1H- pyrazol-4-yl)phenyl]-2- (5-tetrahydrofuran-3-yl- 4H-1,2,4-triazol-3- yl)propanamide | 2.04 | 461.266 |

General Method E: The bromotriazole of Preparation 90 (0.13-0.27 mmol) in dioxane (2 mL) and water (0.1 mL) was degassed in a sealed tube for 30 minutes. The appropriate commercially available boronic acid or ester (1.4-3 equivalents), Pd(dppf)Cl₂·DCM (5 mol %) and cesium carbonate (3 equivalents) were added and the reaction mixture was stirred at 120° C. for 16 hours. The reaction mixture was then diluted with water and extracted with EtOAc (2×50 mL), dried over Na₂SO₄, filtered and concentrated under reduced pressure giving the crude intermediate compound. SEM-deprotection was carried out as for method A.

The examples listed in the table below were all accessed using General Method E.

| Ex. | | | LCMS | Mass |
|---|---|---|---|---|
| No. | Structure | Name | RT | ion |
| 63 | 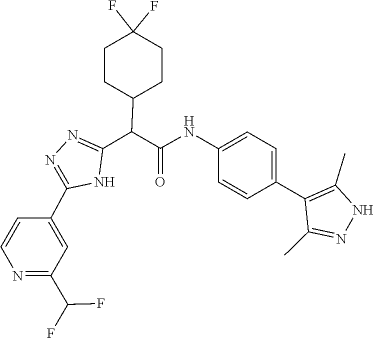 [SMILES: Cc1n[nH]c(C)c1-c1ccc(NC(=O)C(c2nnc(-c3ccnc(C(F)F)c3)[nH]2)C2CCC(F)(F)CC2)cc1] | 2-(4,4-difluorocyclohexyl)-2-[5-[2- (difluoromethyl)-4-pyridyl]-4H- 1,2,4-triazol-3-yl]-N-[4-(3,5- dimethyl-1H-pyrazol-4- yl)phenyl]acetamide | 1.93 | 542.35 |
| 64 | 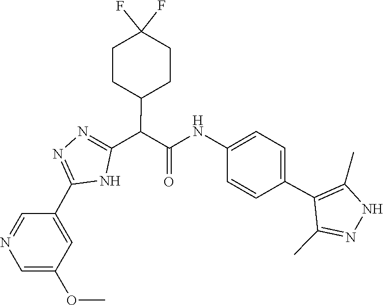 [SMILES: COc1cncc(-c2nnc(C(C(=O)Nc3ccc(-c4c(C)n[nH]c4C)cc3)C3CCC(F)(F)CC3)[nH]2)c1] | 2-(4,4-difluorocyclohexyl)-N-[4- (3,5-dimethyl-1H-pyrazol-4- yl)phenyl]-2-[5-(5-methoxy-3- pyridyl)-4H-1,2,4-triazol-3- yl]acetamide | 1.98 | 522.41 |
| 65 | 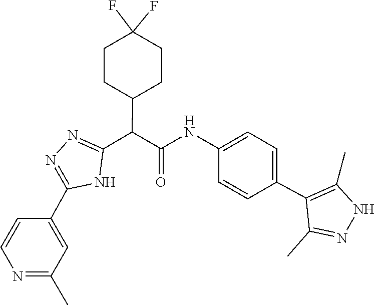 [SMILES: Cc1cc(-c2nnc(C(C(=O)Nc3ccc(-c4c(C)n[nH]c4C)cc3)C3CCC(F)(F)CC3)[nH]2)ccn1] | 2-(4,4-difluorocyclohexyl)-N-[4- (3,5-dimethyl-1H-pyrazol-4- yl)phenyl]-2-[5-(2-methyl-4- pyridyl)-4H-1,2,4-triazol-3- yl]acetamide | 1.67 | 504.37 |
| 66 | 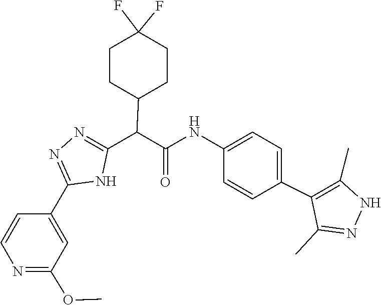 [SMILES: COc1cc(-c2nnc(C(C(=O)Nc3ccc(-c4c(C)n[nH]c4C)cc3)C3CCC(F)(F)CC3)[nH]2)ccn1] | 2-(4,4-difluorocyclohexyl)-N-[4- (3,5-dimethyl-1H-pyrazol-4- yl)phenyl]-2-[5-(2-methoxy-4- pyridyl)-4H-1,2,4-triazol-3- yl]acetamide | 1.91 | 522.39 |

Method F: The appropriate commercially available boronic acid or ester (2-3 equivalents) were added to the bromoimidazole of Preparation 93 (0.034 mmol) in DMF (0.5-1.0 mL) and potassium carbonate (200 mg/mL in water, 3-5 equivalents) and the reaction mixture was degassed with nitrogen for 10 minutes. Pd(dppf)Cl₂·DCM (10 mol %) was added and the reaction mixture was stirred at 90° C. for 30 minutes. The cooled reaction mixture was filtered through a PTFE filter and purified directly by prep. acidic HPLC to afford the desired intermediate SEM protected compound. SEM-deprotection was carried out as for method A.

The examples listed in the table below were all accessed using General Method F.

| Ex. | | | LCMS | Mass |
|---|---|---|---|---|
| No. | Structure | Name | RT | ion |
| 67 | 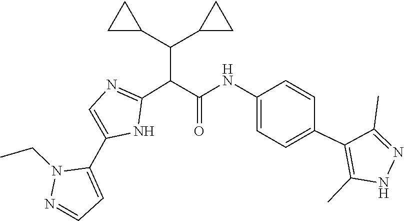 [SMILES: CCn1nccc1-c1cnc(C(C(=O)Nc2ccc(-c3c(C)n[nH]c3C)cc2)C(C2CC2)C2CC2)[nH]1] | 3,3-dicyclopropyl-N-[4-(3,5- dimethyl-1H-pyrazol-4-yl)phenyl]-2- [5-(2-ethylpyrazol-3-yl)-1H- imidazol-2-yl]propanamide | 2.00 | 484.282 |
| 68 | 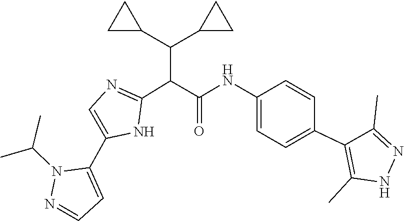 [SMILES: Cc1n[nH]c(C)c1-c1ccc(NC(=O)C(c2ncc(-c3ccnn3C(C)C)[nH]2)C(C2CC2)C2CC2)cc1] | 3,3-dicyclopropyl-N-[4-(3,5- dimethyl-1H-pyrazol-4-yl)phenyl]-2- [5-(2-isopropylpyrazol-3-yl)-1H- imidazol-2-yl]propanamide | 2.05 | 498.298 |
| 69 | 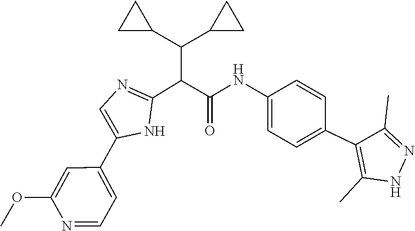 [SMILES: COc1cc(-c2cnc(C(C(=O)Nc3ccc(-c4c(C)n[nH]c4C)cc3)C(C3CC3)C3CC3)[nH]2)ccn1] | 3,3-dicyclopropyl-N-[4-(3,5- dimethyl-1H-pyrazol-4-yl)phenyl]-2- [5-(2-methoxy-4-pyridyl)-1H- imidazol-2-yl]propanamide | 2.10 | 497.266 |

### Example 70: 3,3-dicyclopropyl-N-[4-(3,5-dimethyl-1H-pyrazol-4-yl)phenyl]-2-[5-(2-isopropylpyrazol-3-yl)-4H-1,2,4-triazol-3-yl]propanamide Enantiomer 1 and

### Example 71: 3,3-dicyclopropyl-N-[4-(3,5-dimethyl-1H-pyrazol-4-yl)phenyl]-2-[5-(2-isopropylpyrazol-3-yl)-4H-1,2,4-triazol-3-yl]propanamide Enantiomer 2

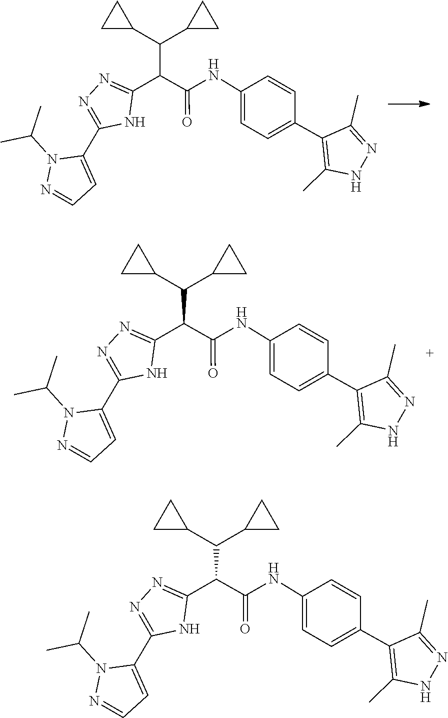 [SMILES: Cc1n[nH]c(C)c1-c1ccc(NC(=O)C(c2nnc(-c3ccnn3C(C)C)[nH]2)C(C2CC2)C2CC2)cc1 ; Cc1n[nH]c(C)c1-c1ccc(NC(=O)[C@@H](c2nnc(-c3ccnn3C(C)C)[nH]2)C(C2CC2)C2CC2)cc1 ; Cc1n[nH]c(C)c1-c1ccc(NC(=O)[C@H](c2nnc(-c3ccnn3C(C)C)[nH]2)C(C2CC2)C2CC2)cc1]

The compound of Example 43 (20 mg, 0.04 mmol) was subjected to chiral prep. SFC (AD-H column, flow=15 ml/min, makeup flow=5 ml/min, gas regulator set to 80 psi, iPrOH) to afford the title compounds.
Enantiomer 1; 8 mg, (AD-H, 23% iPrOH isocratic) RT=3.47 min
Enantiomer 2; 7 mg, (AD-H, 23% iPrOH isocratic) RT=5.89 min

### Example 8: IL-8 Release Assay in Human Epithelial Keratinocytes Adult (HEKa)

Keratinocytes were seeded at 3500 cells/well in 384-well ViewPlates (Perkin Elmer) in Epilife medium (Thermo Fisher) containing human keratinocyte growth supplement (HKGS) without hydrocortisone and incubated in a humid incubator at 37° C., 5% CO₂, overnight. The following day growth medium was removed and 25 μl fresh Epilife medium was added. 75 nL test compound in100% DMSO was added into each well reserved for test compounds, by the use of acoustic pipetting. The remaining wells received an equal volume of DMSO only, as vehicle control, or terfenadine in DMSO, as a positive control for any cytotoxic compounds. Subsequently, another 25 μL Epilife medium was added to each well. Finally, wells containing test compounds and wells prepared to yield maximum stimulation received 25 μL of 9 ng/mL recombinant, human embryonic kidney cell (HEK)-derived human IL-17AA+30 ng/mL human TNF-alpha, in Epilife medium. Wells prepared to define 100% inhibition of IL-17 effects received 25 μL of 30 ng/mL human TNF-alpha alone, in Epilife medium. Final concentrations were 3 ng/mL HEK-human IL-17AA+10 ng/mL human TNFalpha (maximum stimulation) and 10 ng/mL human TNFalpha alone (100% inhibition, Emax), respectively. Cells were incubated for 68-72 hours in the incubator. IL-8 released from the cells was measured by the use of a commercial homogenous time-resolved fluorescence (HTRF) assay (CisBio). 2 μL cell culture supernatant was transferred to a 384-well Proxiplate. 5 μL HTRF reagent was added and the plates were incubated sealed in the dark for 3-22 hours at room temperature. Time-resolved fluorescence was read at 665 vs 620 nm, with excitation at 320 nm, and IL-8 levels were calculated as percent of controls. Reduction of the amount of secreted IL-8 indicates decreased IL-17 signaling. Concentration response curves were fitted by the use of a four-parameter logistic equation. Relative IC₅₀ and Emax were reported from curves showing acceptable fit (r²>0.9). Cytotoxicity was measured in the cell-containing Viewplates following addition of 7 μL PrestoBlue (Thermo Fisher) and incubation for 2.5-3 hours at room temperature, by measuring fluorescence at 615 nm (excitation at 535 nm). Fluorescence was directly proportional to the amount of metabolic activity. Reduction of fluorescence signal indicated cytotoxicity.

Compounds of the present invention were tested in the IL-8 release assay in human epithelial keratinocytes. The results are summarized in Table 1.

TABLE 1

| | Rel EC₅₀ IL-8 |
|---|---|
| Example No. | release assay |
| 1 | 290 |
| 2 | 310 |
| 3 | 3100 |
| 4 | 2900 |
| 5 | 390 |
| 6 | 1300 |
| 7 | 800 |
| 9 | 450 |
| 10 | 1900 |
| 11 | 93 |
| 12 | 140 |
| 13 | 170 |
| 14 | 300 |
| 15 | 350 |
| 16 | 1500 |
| 17 | 240 |
| 18 | 310 |
| 19 | 620 |
| 20 | 990 |
| 21 | 200 |
| 22 | 430 |
| 23 | 59 |
| 24 | 690 |
| 25 | 230 |
| 26 | 24 |
| 27 | 32 |
| 28 | 140 |
| 29 | 1400 |
| 30 | 270 |
| 31 | 430 |
| 32 | 910 |
| 33 | 480 |
| 34 | 430 |
| 35 | 160 |
| 36 | 440 |
| 37 | 380 |
| 38 | 360 |
| 39 | 500 |
| 40 | 860 |
| 41 | >3250 |
| 42 | 470 |
| 43 | 66 |
| 44 | 77 |
| 45 | 22 |
| 46 | no |
| 47 | 83 |
| 48 | 130 |
| 49 | 460 |
| 50 | 110 |
| 51 | 100 |
| 52 | 180 |
| 53 | 73 |
| 54 | 76 |
| 55 | 27 |
| 56 | 320 |
| 57 | 59 |
| 58 | 310 |
| 59 | 45 |
| 60 | 250 |
| 61 | 2700 |
| 62 | 1600 |
| 63 | 200 |
| 64 | Not tested |
| 65 | 520 |
| 66 | 640 |
| 67 | 210 |
| 68 | 170 |
| 69 | 490 |
| 70 | 27 |
| 71 | >625 nM |
| 72 | 260 |

The following are further embodiments of the invention:

Embodiment 1. A compound having the formula (I)

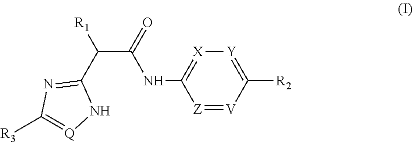

wherein X, Y, Z and V are each independently selected from N, CH and C(R₄);

R₄ is selected from (C₁-C₆)alkyl, (C₁-C₆)alkoxy and halogen, wherein said (C₁-C₆)alkyl and (C₁-C₆)alkoxy is optionally substituted with one of more substituents independently selected from halogen;

Q is C(R₅), or N;

R₁ is selected from the group consisting of —CHR₆R₇, (C₃-C₁₀)cycloalkyl and G, wherein said (C₃-C₁₀)cycloalkyl and G are optionally substituted with one or more substituents independently selected from deuterium, halogen, cyano, (C₁-C₄)alkyl and halo(C₁-C₄)alkyl;

G is

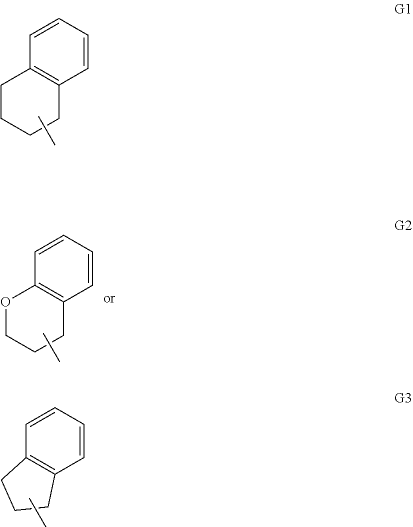

R₆ and R₇ each independently represent hydrogen, phenyl, (C₁-C₆)alkyl, (C₃-C₇)cycloalkyl, or (C₃-C₇)cycloalkyl-(C₁-C₆)alkyl, wherein said phenyl, (C₁-C₆)alkyl, (C₃-C₇)cycloalkyl and (C₃-C₇)cycloalkyl-(C₁-C₆)alkyl, is optionally substituted with one or more substituents independently selected from halogen, cyano, and (C₁-C₄)alkyl; with the proviso that at least one of R₆and R₇ are different from hydrogen;

R₂ is selected from the group consisting of 5- or 6-membered heteroaryl, wherein said 5-or 6-membered heteroaryl is optionally substituted with one or more substituents independently selected from Rₐ, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

L is selected from the group consisting of a bond or —CHR_{g}O—,

R_{g} is independently selected from hydrogen and (C₁-C₆)alkyl;

R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b};

R_{b} is deuterium, halogen, hydroxy, —NR_{c}R_{d}, (C₁-C₆)alkyl, (C₁-C₆)alkylcarbonyl, (C₃-C₇)cycloalkyl, phenyl, 5- or 6-membered heteroaryl or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy,

or pharmaceutically acceptable salts, hydrates and solvates thereof.

Embodiment 2. The compound according to embodiment 1 having the formula (Ia)

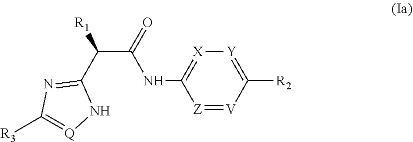

wherein X, Y, Z, V, Q, R₁, R₂ and R₃ are as defined in embodiment 1 or pharmaceutically acceptable salts, hydrates and solvates thereof.

Embodiment 3. The compound according to embodiment 1 having the formula (Ib)

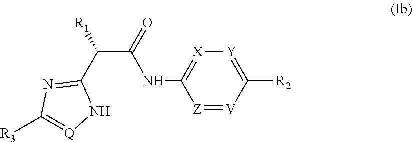

wherein X, Y, Z, V, Q, R₁, R₂ and R₃ are as defined in embodiment 1 or pharmaceutically acceptable salts, hydrates and solvates thereof.

Embodiment 4. The compound according to any one of embodiments 1-3, wherein R₂ is selected from pyrazolyl and imidazolyl, wherein said pyrazolyl or imidazolyl is optionally substituted with one or more substituents independently selected from (C₁-C₆)alkyl.

Embodiment 5. The compound according to any one of embodiments 1-4, wherein R₂ is 3,5-di(C₁-C₆)alkyl-pyrazol-4-yl or 3,5-di(C₁-C₆)alkyl-imidazol-4-yl.

Embodiment 6. The compound according to any one of embodiments 1-3 above, wherein R₂ is selected from 3,5-di(C₁-C₆)alkyl-pyrazol-4-yl or 3,5-di(C₁-C₆)alkyl-imidazol-4-yl, wherein said 3,5-di(C₁-C₆)alkyl-pyrazol-4-yl or 3,5-di(C₁-C₆)alkyl-imidazol-4-yl contain a nitrogen ring atom substituted by a substituent selected from -L-PO(OH)₂.

Embodiment 7. The compound according to any one of embodiments 1-6, wherein

R₃ is selected from —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more substituents independently selected from R_{b}.

Embodiment 8. The compound according to any one of embodiments 1-7, wherein Q is C(R₅) and R₅ is selected from hydrogen, halogen, hydroxy, (C₁-C₆)alkyl, (C₃-C₇)cycloalkyl, (C₃-C₇)cycloaIkoxy, (C₁-C₆)alkoxy.

Embodiment 9. The compound according to any one of claims 1-7, wherein Q is N.

Embodiment 10. The compound according to any one of embodiments 1-9, wherein X, Y, Z and V are independently selected from CH and C(R₄).

Embodiment 11. The compound according to any one of embodiments 1-9, wherein X is N and Y, Z and V are independently selected from CH and C(R₄).

Embodiment 12. The compound according to any one of embodiments 1-9, wherein Y is N and X, Z and V are independently selected from CH and C(R₄).

Embodiment 13. The compound according to any one of embodiments 1-9, wherein X and Y are N and V and Z are independently selected from CH and C(R₄).

Embodiment 14. The compound according to any one of embodiments 1-9, wherein Y and Z are N and X and V are independently selected from CH and C(R₄).

Embodiment 15. The compound according to any one of embodiments 1-9, wherein X and Z are N and Y and V are independently selected from CH and C(R₄).

Embodiment 16. The compound according to any one of embodiments 1-9, wherein Y and V are N and X and Z are independently selected from CH and C(R₄).

Embodiment 17. The compound according to any one of embodiments 1-9 , wherein X is N, Y is C(R₄) and V and Z are CH.

Embodiment 18. The compound according to any one of the embodiments above, wherein R₁ is selected from —CHR₆R₇, and wherein R₆ and R₇ each independently represent (C₃-C₇)cycloalkyl, wherein said (C₃-C₇)cycloalkyl is optionally substituted with one or more substituents independently selected from halogen, cyano, and (C₁-C₄)alkyl.

Embodiment 19. The compound according to any one of the embodiments above wherein R₁ is selected from —CHR₆R₇, and wherein R₆ and R₇ each independently represent (C₃-C₄)cycloalkyl, wherein said (C₃-C₄)cycloalkyl is optionally substituted with one or more substituents independently selected from halogen, cyano, and (C₁-C₄)alkyl.

Embodiment 20. The compound according to embodiment 19 above wherein R₁ is selected from —CHR₆R₇, and wherein R₆ and R₇ each independently represent cyclopropyl or cyclobutyl.

Embodiment 21. The compound according to embodiment 20 above wherein R₁ is selected from —CHR₆R₇, and wherein R₆ and R₇ are both cyclopropyl.

Embodiment 22. The compound according to embodiment 20 above wherein R₁ is selected from —CHR₆R₇, and wherein R₆ and R₇ are both cyclobutyl.

Embodiment 23. The compound according to any one of the embodiments above wherein R₁ is selected from —CHR₆R₇, and wherein R₆ and R₇ each independently represent (C₃-C₄)cycloalkylmethyl, wherein said (C₃-C₄)cycloalkylmethyl is optionally substituted with one or more substituents independently selected from halogen, cyano, and (C₁-C₄)alkyl.

Embodiment 24. The compound according to embodiment 23 above, wherein R₁ is selected from —CHR₆R₇, and wherein R₆ and R₇ are both cyclopropylmethyl.

Embodiment 25. The compound according to embodiment 23 above, wherein R₁ is selected from —CHR₆R₇, and wherein R₆ and R₇ are both cyclobutylmethyl.

Embodiment 26. The compound according to any one of the embodiments above, wherein R₁ is (C₅-C₇)cycloalkyl optionally substituted with one or more (C₁-C₄)alkyl.

Embodiment 27. The compound according to any one of the embodiments above, wherein R₁ is cyclohexyl optionally substituted with one or more (C₁-C₄)alkyl.

1. A compound having the formula (I) 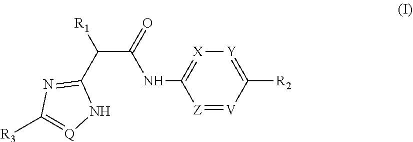
wherein:
X, Y, Z, and V are each independently selected from N, CH, and C(R₄);
R₄ is selected from (C₁-C₆)alkyl, (C₁-C₆)alkoxy, amino, hydroxy, and halogen, wherein said (C₁-C₆)alkyl and (C₁-C₆)alkoxy are optionally substituted with one of more halogens;
Q is C(R₅), or N;
R₁ is selected from the group consisting of —CHR₆R₇, (C₃-C₁₀)cycloalkyl, and G, wherein said (C₃-C₁₀)cycloalkyl and G are optionally substituted with one or more substituents independently selected from deuterium, halogen, cyano, (C₁-C₄)alkyl, and halo(C₁-C₄)alkyl;
G is 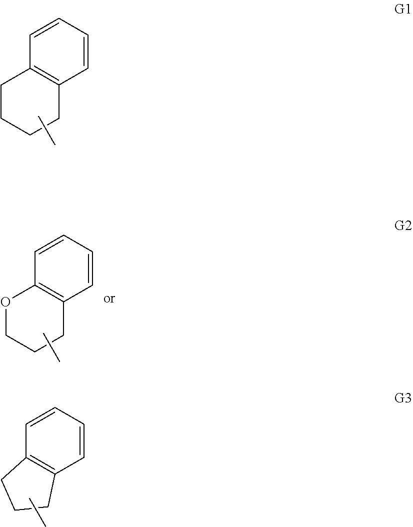
R₆ and R₇ each independently is selected from the group consisting of hydrogen, phenyl, (C₁-C₆)alkyl, (C₃-C₇)cycloalkyl, and (C₃-C₇)cycloalkyl-(C₁-C₆)alkyl, wherein said phenyl, (C₁-C₆)alkyl, (C₃-C₇)cycloalkyl, and (C₃-C₇)cycloalkyl-(C₁-C₆)alkyl, are optionally substituted with one or more substituents independently selected from halogen, cyano, and (C₁-C₄)alkyl; with the proviso that at least one of R₆ and R₇ is not hydrogen;
R₂ is a 5- or 6-membered heteroaryl, wherein said 5- or 6-membered heteroaryl is optionally substituted with one or more Rₐ, wherein said 5- or 6-membered heteroaryl may optionally contain —CO— as a ring member and wherein when said 5-membered heteroaryl contains nitrogen as a ring atom and said nitrogen may optionally be substituted with -L-PO(OH)₂;
Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
L is a bond or —CHR_{g}O—,
R_{g} is hydrogen or (C₁-C₆)alkyl;
R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more R_{b};
R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone, or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl, or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂—, and —NR_{c}R_{d};
R_{c} and R_{d} each independently are selected from the group consisting of hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy,
or a pharmaceutically acceptable salt, hydrate, or solvate thereof.

2. The compound according to claim 1 having the formula (la) 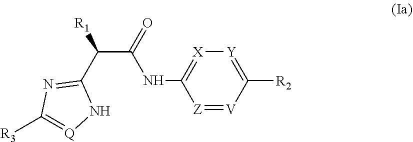
or a pharmaceutically acceptable salt, hydrate, or solvate thereof.

3. The compound according to claim 1, wherein X, Y, Z, and V are each independently selected from N, CH, and C(R₄);
R₄ is selected from (C₁-C₆)alkyl, (C₁-C₆)alkoxy, amino, hydroxy, and halogen, wherein said (C₁-C₆)alkyl and (C₁-C₆)alkoxy are optionally substituted with one of more substituents independently selected from halogen;
Q is C(R₅) or N;
R₁ is —CHR₆R₇, and wherein R₆ and R₇ each independently is selected from the group consisting of hydrogen, phenyl, cyclopropyl, cyclobutyl, cyclopentyl, cyclohexyl, cycloheptyl, cyclopropylmethyl, cyclobutylmethyl, methyl, and ethyl, wherein said phenyl, cyclopropyl, cyclobutyl, cyclopentyl, cyclohexyl, cyclopropylmethyl, cyclobutylmethyl, methyl, and ethyl are optionally substituted with one or more substituents independently selected from halogen, cyano, and (C₁-C₄)alkyl; with the proviso that at least one of R₆ and R₇ is not hydrogen;
R₂ is a 5- or 6-membered heteroaryl, wherein said 5- or 6-membered heteroaryl is optionally substituted with one or more Rₐ, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;
Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
L is a bond or —CHR_{g}O—,
R_{g} is hydrogen or (C₁-C₆)alkyl;
R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl are optionally substituted with one or more substituents independently selected from R_{b};
R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone, or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl, or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂—, and —NR_{c}R_{d};
R_{c} and R_{d} each independently are selected from hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy,
or a pharmaceutically acceptable salt, hydrate, or solvate thereof.

4. The compound according to claim 1, wherein:
X, Y, Z, and V are each independently selected from N, CH, and C(R₄);
R₄ is selected from (C₁-C₆)alkyl, (C₁-C₆)alkoxy, amino, hydroxy, and halogen, wherein said (C₁-C₆)alkyl and (C₁-C₆)alkoxy are optionally substituted with one of more halogens;
Q is C(R₅) or N;
R₁ is —CHR₆R₇, and wherein R₆ and R₇ each independently is selected from (C₃-C₇)cycloalkyl and (C₃-C₇)cycloalkyl-(C₁-C₆)alkyl, wherein said (C₃-C₇)cycloalkyl and (C₃-C₇)cycloalkyl-(C₁-C₆)alkyl are optionally substituted with one or more substituents independently selected from halogen, cyano, and (C₁-C₄)alkyl;
R₂ is a 5- or 6-membered heteroaryl, wherein said 5- or 6-membered heteroaryl is optionally substituted with one or more Rₐ, wherein said 5- or 6-membered heteroaryl may optionally contain —CO— as a ring member and wherein when said 5-membered heteroaryl contains nitrogen as a ring atom said nitrogen may optionally be substituted with -L-PO(OH)₂;
Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
L is a bond or —CHR_{g}O—,
R_{g} is hydrogen or (C₁-C₆)alkyl;
R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more R_{b};
R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridine, or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl, or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂—, and —NR_{c}R_{d};
R_{c} and R_{d} each independently are selected from the group consisting of hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy,
or a pharmaceutically acceptable salt, hydrate, or solvate thereof.

5. The compound according to claim 4, wherein:
R₁ is —CHR₆R₇, and wherein R₆ and R₇ each is (C₃-C₇)cycloalkyl, wherein said (C₃-C₇)cycloalkyl is optionally substituted with one or more substituents independently selected from halogen, cyano, and (C₁-C₄)alkyl.

6. The compound according to claim 1, wherein X, Y, Z, and V are each independently selected from N, CH, and C(R₄); R₄ is selected from (C₁-C₆)alkyl, (C₁-C₆)alkoxy, amino, hydroxy, and halogen, wherein said (C₁-C₆)alkyl and (C₁-C₆)alkoxy is optionally substituted with one of more halogens;
Q is C(R₅) or N;
R₁ is selected from cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl, or spiro[2.5]octanyl, wherein said cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl, or spiro[2.5]octanyl is optionally substituted with one or more substituents independently selected from deuterium, halogen, cyano, (C₁-C₄)alkyl, and halo(C₁-C₄)alkyl;
R₂ is a 5- or 6-membered heteroaryl, wherein said 5- or 6-membered heteroaryl is optionally substituted with one or more Rₐ, wherein said 5- or 6-membered heteroaryl may optionally contain —CO— as a ring member and wherein when said 5-membered heteroaryl contains nitrogen as a ring atom said nitrogen may optionally be substituted with -L-PO(OH)₂;
Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
L is a bond or —CHR_{g}O—,
R_{g} is hydrogen or (C₁-C₆)alkyl;
R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more R_{b};
R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone, or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl, or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂—, and —NR_{c}R_{d};
R_{c} and R_{d} each independently are selected from hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl or piperidinyl is optionally substituted with one or more substituents independently selected from halogen, cyano, and hydroxy,
or a pharmaceutically acceptable salt, hydrate, or solvate thereof.

7. The compound according to claim 6, wherein R₁ is cyclohexyl optionally substituted with one or more (C₁-C₄)alkyl.

8. The compound according to claim 1, wherein:
X, Y, Z, and V are each independently selected from N, CH, and C(R₄); R₄ is selected from (C₁-C₆)alkyl, (C₁-C₆)alkoxy, amino, hydroxy, and halogen, wherein said (C₁-C₆)alkyl and (C₁-C₆)alkoxy is optionally substituted with one of more halogens;
Q is C(R₅) or N;
R₁ is 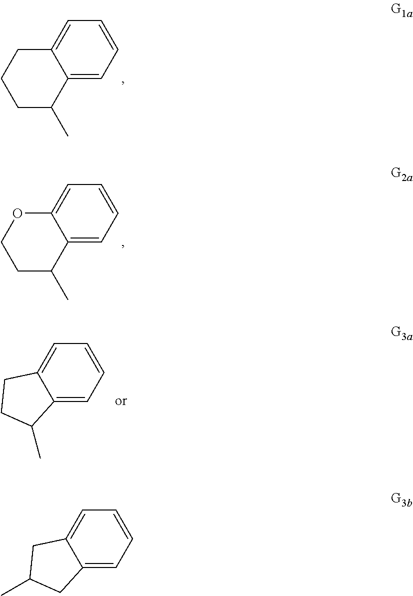
wherein said G₁ₐ, G₂ₐ, G₃ₐ, G_{3b} are optionally substituted with one or more substituents independently selected from deuterium, halogen, cyano, (C₁-C₄)alkyl, and halo(C₁-C₄)alkyl.
R₂ is a 5- or 6-membered heteroaryl, wherein said 5- or 6-membered heteroaryl is optionally substituted with one or more Rₐ, wherein said 5- or 6-membered heteroaryl may optionally contain —CO— as a ring member and wherein when said 5-membered heteroaryl contains nitrogen as a ring atom said nitrogen may optionally be substituted with -L-PO(OH)₂;
Rₐ is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
L is a bond or —CHR_{g}O—,
R_{g} is hydrogen or (C₁-C₆)alkyl;
R₃ and R₅ are each independently selected from hydrogen, halogen, hydroxy, —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more R_{b};
R_{b} is deuterium, halogen, hydroxy, —NR_{c}R_{d}, (C₁-C₆)alkyl, (C₁-C₆)alkoxy, (C₁-C₆)alkylcarbonyl, (C₃-C₇)cycloalkyl, phenyl, 5- or 6-membered heteroaryl, pyridone, or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl, or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂—, and —NR_{c}R_{d};
R_{c} and R_{d} each independently are selected from hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy,
or a pharmaceutically acceptable salt, hydrate, or solvate thereof.

9. The compound according to claim 1, wherein R₂ is pyrazolyl or imidazolyl, wherein said pyrazolyl or imidazolyl is optionally substituted with one or more from (C₁-C₆)alkyl.

10. The compound according to claim 9, wherein R₂ is 3,5-di(C₁-C₆)alkyl-pyrazol-4-yl or 3,5-di(C₁-C₆)alkyl-imidazol-4-yl.

11. The compound according to claim 1, wherein R₂ is selected from 3,5-di(C₁-C₆)alkyl-pyrazol-4-yl or 3,5-di(C₁-C₆)alkyl-imidazol-4-yl, wherein said 3,5-di(C₁-C₆)alkyl-pyrazol-4-yl or 3,5-di(C₁-C₆)alkyl-imidazol-4-yl contain a nitrogen ring atom substituted by -L-PO(OH)₂.

12. The compound according to claim 1, wherein:
R₃ is —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more R_{b}; and
Q is Nor C(R₅); R₅ is selected from hydrogen, halogen, hydroxy, (C₁-C₆)alkyl, (C₃-C₇)cycloalkyl, (C₃-C₇)cycloalkoxy, and (C₁-C₆)alkoxy.

13. The compound according to claim 12, wherein:
R₃ is selected from —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more R_{b}; and
Q is C(R₅) and R₅ is selected from hydrogen, halogen, hydroxy, (C₁-C₆)alkyl, (C₃-C₇)cycloalkyl, (C₃-C₇)cycloalkoxy, and (C₁-C₆)alkoxy.

14. The compound according to claim 12, wherein:
R₃ is selected from —NR_{c}R_{d}, (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl, wherein said (C₁-C₆)alkyl, (C₃-C₇)cycloalkyl, (C₃-C₇)cycloalkoxy, (C₁-C₆)alkoxy, phenyl, phenoxy, 5-6-membered heteroaryl, and 4-7-membered heterocycloalkyl is optionally substituted with one or more R_{b}; and
Q is N.

15. The compound according to claim 1, wherein:
X, Y, Z, and V are independently selected from CH and C(R₄),
X is N, and Y, Z, and V are independently selected from CH and C(R₄),
Y is N, and X, Z, and V are independently selected from CH and C(R₄),
X and Y are N, and V and Z are independently selected from CH and C(R₄),
Y and Z are N, and X and V are independently selected from CH and C(R₄),
X and Z are N, and Y and V are independently selected from CH and C(R₄), or
Y and V are N, and X and Z are independently selected from CH and C(R₄).

16. The compound according to claim 15, wherein X is N, Y is C(R₄), and V and Z are CH.

17. The compound according to claim 1, wherein:
wherein X, Y, Z, and V is CH;
Q is N;
R₁ is —CHR₆R₇,
R₆ and R₇ each independently is (C₃-C₇)cycloalkyl, or (C₃-C₇)cycloalkyl-(C₁-C₆)alkyl;
R₂ is 3,5-di-((C₁-C₆)alkyl)-1H-pyrazol-4-yl;
R₃ is 4H-1,2,4-triazol-3-yl, wherein said 4H-1,2,4-triazol-3-yl is optionally substituted with one or more R_{b};
R_{b} is (C₁-C₆)alkyl or (C₃-C₇)cycloalkyl, wherein said (C₁-C₆)alkyl or (C₃-C₇)cycloalkyl independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂—, and —NR_{c}R_{d};
R_{c} and R_{d} each independently are selected from hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano and hydroxy,
or a pharmaceutically acceptable salt, hydrate, or solvate thereof.

18. The compound according to claim 1 selected from
i) 3,3-dicyclopropyl-N-[4-(3,5-dimethyl-1H-pyrazol-4-yl)phenyl]-2-(5-phenyl-4H-1,2,4-triazol-3-yl)propanamide;
ii) 3,3-dicyclopropyl-N-[4-(3,5-dimethyl-1H-pyrazol-4-yl)phenyl]-2-(4-phenyl-1H-imidazol-2-yl)propanamide;
iii) 3,3-dicyclopropyl-N-[4-(3,5-dimethyl-1H-pyrazol-4-yl)phenyl]-2-(1H-imidazol-2-yl)propanamide;
iv) 3,3-dicyclopropyl-N-[4-(3,5-dimethyl-1H-pyrazol-4-yl)phenyl]-2-(4H-1,2,4-triazol-3-yl)propanamide;
v) 3,3-dicyclopropyl-N-[4-(3,5-dimethyl-1H-pyrazol-4-yl)phenyl]-2-[5-(2-fluorophenyl)-4H-1,2,4-triazol-3-yl]propanamide;
vi) 2-(5-chloro-4-phenyl-1H-imidazol-2-yl)-3,3-dicyclopropyl-N-[4-(3,5-dimethyl-1H-pyrazol-4-yl)phenyl]propanamide;
vii) 3,3-dicyclopropyl-N-[4-(3,5-dimethyl-1H-pyrazol-4-yl)phenyl]-2-(5-methyl-4-phenyl-1H-imidazol-2-yl)propanamide;
viii) 3,3-dicyclopropyl-N-[4-(3,5-dimethyl-1H-pyrazol-4-yl)phenyl]-2-[5-(2-methoxyphenyl)-1H-imidazol-2-yl]propanamide;
ix) 2-(5-cyclopentyl-4H-1,2,4-triazol-3-yl)-3,3-dicyclopropyl-N-[4-(3,5-dimethyl-1H-pyrazol-4-yl)phenyl]propanamide;
x) 3,3-dicyclopropyl-N-[5-(3,5-dimethyl-1H-pyrazol-4-yl)-6-fluoro-2-pyridyl]-2-[5-(2-isopropylpyrazol-3-yl)-4H-1,2,4-triazol-3-yl]propanamide;
xi) 3,3-dicyclopropyl-N-[4-(3,5-dimethyl-1H-pyrazol-4-yl)-2-fluoro-phenyl]-2-[5-(2-isopropylpyrazol-3-yl)-4H-1,2,4-triazol-3-yl]propanamide;
xii) 3,3-dicyclopropyl-N-[4-(3,5-dimethyl-1H-pyrazol-4-yl)phenyl]-2-[5-(4-methyl-1,2,5-oxadiazol-3-yl)-4H-1,2,4-triazol-3-yl]propanamide;
xiii) 3,3-dicyclopropyl-N-[4-(3,5-dimethyl-1H-pyrazol-4-yl)phenyl]-2-[5-(4-fluoro-2-methyl-pyrazol-3-yl)-4H-1,2,4-triazol-3-yl]propanamide;
xiv) 3,3-dicyclopropyl-N-[4-(3,5-dimethyl-1H-pyrazol-4-yl)phenyl]-2-[5-(3-pyridylmethyl)-4H-1,2,4-triazol-3-yl]propanamide;
xv) 3,3-dicyclopropyl-N-[4-(3,5-dimethyl-1H-pyrazol-4-yl)phenyl]-2-[5-(1H-pyrazol-3-ylmethyl)-4H-1,2,4-triazol-3-yl]propanamide;
xvi) 2-(5-benzyl-4H-1,2,4-triazol-3-yl)-3,3-dicyclopropyl-N-[4-(3,5-dimethyl-1H-pyrazol-4-yl)phenyl]propanamide;
xvii) 2-[5-(2-chlorophenyl)-4H-1,2,4-triazol-3-yl]-3,3-dicyclopropyl-N-[4-(3,5-dimethyl-1H-pyrazol-4-yl)phenyl]propanamide;
xviii) 3,3-dicyclopropyl-N-[4-(3,5-dimethyl-1H-pyrazol-4-yl)phenyl]-2-[5-(2-methoxyphenyl)-4H-1,2,4-triazol-3-yl]propanamide;
xix) 3,3-dicyclopropyl-N-[4-(3,5-dimethyl-1H-pyrazol-4-yl)phenyl]-2-[5-(1-methylpyrazol-4-yl)-4H-1,2,4-triazol-3-yl]propanamide;
xx) 3,3-dicyclopropyl-N-[4-(3,5-dimethyl-1H-pyrazol-4-yl)phenyl]-2-[5-(2-methylpyrazol-3-yl)-4H-1,2,4-triazol-3-yl]propanamide;
xxi) 3,3-dicyclopropyl-N-[4-(3,5-dimethyl-1H-pyrazol-4-yl)phenyl]-2-[5-(1-methyl-2-oxo-4-pyridyl)-4H-1,2,4-triazol-3-yl]propanamide;
xxii) 3,3-dicyclopropyl-N-[4-(3,5-dimethyl-1H-pyrazol-4-yl)phenyl]-2-[5-(2-ethylpyrazol-3-yl)-4H-1,2,4-triazol-3-yl]propanamide;
xxiii) 3,3-dicyclopropyl-N-[4-(3,5-dimethyl-1H-pyrazol-4-yl)phenyl]-2-[5-(1H-pyrazol-5-yl)-4H-1,2,4-triazol-3-yl]propanamide;
xxiv) 3,3-dicyclopropyl-N-[4-(3,5-dimethyl-1H-pyrazol-4-yl)phenyl]-2-[5-(1,3-dimethylpyrazol-4-yl)-4H-1,2,4-triazol-3-yl]propanamide;
xxv) 3,3-dicyclopropyl-N-[4-(3,5-dimethyl-1H-pyrazol-4-yl)phenyl]-2-[5-[2-(2,2,2-trifluoroethyl)pyrazol-3-yl]-4H-1,2,4-triazol-3-yl]propanamide;
xxvi) 2-[5-(2-cyclobutylpyrazol-3-yl)-4H-1,2,4-triazol-3-yl]-3,3-dicyclopropyl-N-[4-(3,5-dimethyl-1H-pyrazol-4-yl)phenyl]propanamide;
xxvii) 3,3-dicyclopropyl-N-[4-(3,5-dimethyl-1H-pyrazol-4-yl)phenyl]-2-[5-(2-methyl-4-pyridyl)-4H-1,2,4-triazol-3-yl]propanamide;
xxviii) 3,3-dicyclopropyl-N-[4-(3,5-dimethyl-1H-pyrazol-4-yl)phenyl]-2-[5-(3,5-dimethyl-1H-pyrazol-4-yl)-4H-1,2,4-triazol-3-yl]propanamide;
xxix) 3,3-dicyclopropyl-N-[4-(3,5-dimethyl-1H-pyrazol-4-yl)phenyl]-2-[5-(1-isopropylpyrazol-4-yl)-4H-1,2,4-triazol-3-yl]propanamide;
xxx) 3,3-dicyclopropyl-N-[4-(3,5-dimethyl-1H-pyrazol-4-yl)phenyl]-2-[5-(3-pyridyl)-4H-1,2,4-triazol-3-yl]propanamide;
xxxi) 3,3-dicyclopropyl-2-[5-(3,5-dimethylisoxazol-4-yl)-4H-1,2,4-triazol-3-yl]-N-[4-(3,5-dimethyl-1H-pyrazol-4-yl)phenyl]propanamide;
xxxii) 3,3-dicyclopropyl-N-[4-(3,5-dimethyl-1H-pyrazol-4-yl)phenyl]-2-[5-(4-pyridyl)-4H-1,2,4-triazol-3-yl]propanamide;
xxxiii) 3,3-dicyclopropyl-N-[4-(3,5-dimethyl-1H-pyrazol-4-yl)phenyl]-2-[5-(6-methoxy-3-pyridyl)-4H-1,2,4-triazol-3-yl]propanamide;
xxxiv) 3,3-dicyclopropyl-N-[4-(3,5-dimethyl-1H-pyrazol-4-yl)phenyl]-2-[5-(2-methoxy-4-pyridyl)-4H-1,2,4-triazol-3-yl]propanamide;
xxxv) 3,3-dicyclopropyl-N-[4-(3,5-dimethyl-1H-pyrazol-4-yl)phenyl]-2-[5-(6-methyl-3-pyridyl)-4H-1,2,4-triazol-3-yl]propanamide;
xxxvi) 3,3-dicyclopropyl-N-[4-(3,5-dimethyl-1H-pyrazol-4-yl)phenyl]-2-[5-(2,4-dimethylpyrazol-3-yl)-4H-1,2,4-triazol-3-yl]propanamide;
xxxvii) 3,3-dicyclopropyl-N-[4-(3,5-dimethyl-1H-pyrazol-4-yl)phenyl]-2-[5-(1,5-dimethylpyrazol-4-yl)-4H-1,2,4-triazol-3-yl]propanamide;
xxxviii) 3,3-dicyclopropyl-N-[4-(3,5-dimethyl-1H-pyrazol-4-yl)phenyl]-2-[5-(3-methyl-1H-pyrazol-4-yl)-4H-1,2,4-triazol-3-yl]propanamide;
xxxix) 3,3-dicyclopropyl-N-[4-(3,5-dimethyl-1H-pyrazol-4-yl)phenyl]-2-[5-(3-methyltriazol-4-yl)-4H-1,2,4-triazol-3-yl]propanamide;
xl) 3,3-dicyclopropyl-N-[4-(3,5-dimethyl-1H-pyrazol-4-yl)phenyl]-2-[5-(5-methylisoxazol-4-yl)-4H-1,2,4-triazol-3-yl]propanamide;
xli) 3,3-dicyclopropyl-N-[4-(3,5-dimethyl-1H-pyrazol-4-yl)phenyl]-2-[5-(3-methylisoxazol-4-yl)-4H-1,2,4-triazol-3-yl]propanamide;
xlii) 3,3-dicyclopropyl-N-[4-(3,5-dimethyl-1H-pyrazol-4-yl)phenyl]-2-[5-(2-isopropylpyrazol-3-yl)-4H-1,2,4-triazol-3-yl]propanamide;
xliii) 3,3-dicyclopropyl-2-[5-(2-cyclopropylpyrazol-3-yl)-4H-1,2,4-triazol-3-yl]-N-[4-(3,5-dimethyl-1H-pyrazol-4-yl)phenyl]propanamide;
xliv) 3,3-dicyclopropyl-2-[5-[2-(cyclopropylmethyl)pyrazol-3-yl]-4H-1,2,4-triazol-3-yl]-N-[4-(3,5-dimethyl-1H-pyrazol-4-yl)phenyl]propanamide;
xlv) 3,3-dicyclopropyl-N-[4-(3,5-dimethyl-1H-pyrazol-4-yl)phenyl]-2-[5-(1,4-dimethylpyrazol-3-yl)-4H-1,2,4-triazol-3-yl]propanamide;
xlvi) 3,3-dicyclopropyl-N-[4-(3,5-dimethyl-1H-pyrazol-4-yl)phenyl]-2-[5-(2,5-dimethylpyrazol-3-yl)-4H-1,2,4-triazol-3-yl]propanamide;
xlvii) 3,3-dicyclopropyl-N-[4-(3,5-dimethyl-1H-pyrazol-4-yl)phenyl]-2-[5-(5-methoxy-3-pyridyl)-4H-1,2,4-triazol-3-yl]propanamide;
xlviii) 3,3-dicyclopropyl-N-[4-(3,5-dimethyl-1H-pyrazol-4-yl)phenyl]-2-[5-(2-methoxy-3-pyridyl)-4H-1,2,4-triazol-3-yl]propanamide;
xlix) 3,3-dicyclopropyl-N-[4-(3,5-dimethyl-1H-pyrazol-4-yl)phenyl]-2-[5-(5-methyl-3-pyridyl)-4H-1,2,4-triazol-3-yl]propanamide;
l) 3,3-dicyclopropyl-N-[4-(3,5-dimethyl-1H-pyrazol-4-yl)phenyl]-2-[5-(2-ethoxy-4-pyridyl)-4H-1,2,4-triazol-3-yl]propanamide;
li) 3,3-dicyclopropyl-N-[4-(3,5-dimethyl-1H-pyrazol-4-yl)phenyl]-2-[5-(2-methyl-3-pyridyl)-4H-1,2,4-triazol-3-yl]propanamide;
lii) 3,3-dicyclopropyl-N-[4-(3,5-dimethyl-1H-pyrazol-4-yl)phenyl]-2-[5-(4-methyl-3-pyridyl)-4H-1,2,4-triazol-3-yl]propanamide;
liii) 3,3-dicyclopropyl-N-[4-(3,5-dimethyl-1H-pyrazol-4-yl)phenyl]-2-[5-[2-(trifluoromethyl)-4-pyridyl]-4H-1,2,4-triazol-3-yl]propanamide;
liv) 3,3-dicyclopropyl-2-[5-[2-(difluoromethyl)-4-pyridyl]-4H-1,2,4-triazol-3-yl]-N-[4-(3,5-dimethyl-1H-pyrazol-4-yl)phenyl]propanamide;
lv) 3,3-dicyclopropyl-N-[4-(3,5-dimethyl-1H-pyrazol-4-yl)phenyl]-2-[5-(4-methoxy-3-pyridyl)-4H-1,2,4-triazol-3-yl]propanamide;
lvi) 3,3-dicyclopropyl-N-[4-(3,5-dimethyl-1H-pyrazol-4-yl)phenyl]-2-[5-[2-(3-hydroxypropyl)pyrazol-3-yl]-4H-1,2,4-triazol-3-yl]propanamide;
lvii) 3,3-dicyclopropyl-N-[4-(3,5-dimethyl-1H-pyrazol-4-yl)phenyl]-2-[5-(2-pyridyl)-4H-1,2,4-triazol-3-yl]propanamide;
lviii) 3,3-dicyclopropyl-N-[4-(3,5-dimethyl-1H-pyrazol-4-yl)phenyl]-2-[5-(6-methyl-2-pyridyl)-4H-1,2,4-triazol-3-yl]propanamide;
lix) 3,3-dicyclopropyl-N-[4-(3,5-dimethyl-1H-pyrazol-4-yl)phenyl]-2-[5-(3-methyl-2-pyridyl)-4H-1,2,4-triazol-3-yl]propanamide; p1 lx) 3,3-dicyclopropyl-N-[4-(3,5-dimethyl-1H-pyrazol-4-yl)phenyl]-2-(5-tetrahydropyran-4-yl-4H-1,2,4-triazol-3-yl)propanamide;
lxi) 3,3-dicyclopropyl-N-[4-(3,5-dimethyl-1H-pyrazol-4-yl)phenyl]-2-(5-tetrahydrofuran-3-yl-4H-1,2,4-triazol-3-yl)propanamide;
lxii) 2-(4,4-difluorocyclohexyl)-2-[5-[2-(difluoromethyl)-4-pyridyl]-4H-1,2,4-triazol-3-yl]-N-[4-(3,5-dimethyl-1H-pyrazol-4-yl)phenyl]acetamide;
lxiii) 2-(4,4-difluorocyclohexyl)-N-[4-(3,5-dimethyl-1H-pyrazol-4-yl)phenyl]-2-[5-(5-methoxy-3-pyridyl)-4H-1,2,4-triazol-3-yl]acetamide;
lxiv) 2-(4,4-difluorocyclohexyl)-N-[4-(3,5-dimethyl-1H-pyrazol-4-yl)phenyl]-2-[5-(2-methyl-4-pyridyl)-4H-1,2,4-triazol-3-yl]acetamide;
lxv) 2-(4,4-difluorocyclohexyl)-N-[4-(3,5-dimethyl-1H-pyrazol-4-yl)phenyl]-2-[5-(2-methoxy-4-pyridyl)-4H-1,2,4-triazol-3-yl]acetamide;
lxvi) 3,3-dicyclopropyl-N-[4-(3,5-dimethyl-1H-pyrazol-4-yl)phenyl]-2-[5-(2-ethylpyrazol-3-yl)-1H-imidazol-2-yl]propanamide;
lxvii) 3,3-dicyclopropyl-N-[4-(3,5-dimethyl-1H-pyrazol-4-yl)phenyl]-2-[5-(2-isopropylpyrazol-3-yl)-1H-imidazol-2-yl]propanamide;
lxviii) 3,3-dicyclopropyl-N-[4-(3,5-dimethyl-1H-pyrazol-4-yl)phenyl]-2-[5-(2-methoxy-4-pyridyl)-1H-imidazol-2-yl]propanamide;
lxix) (2R)-3,3-dicyclopropyl-N-[4-(3,5-dimethyl-1H-pyrazol-4-yl)phenyl]-2-[5-(2-isopropylpyrazol-3-yl)-4H-1,2,4-triazol-3-yl]propanamide;
lxx) (2S)-3,3-dicyclopropyl-N-[4-(3,5-dimethyl-1H-pyrazol-4-yl)phenyl]-2-[5-(2-isopropylpyrazol-3-yl)-4H-1,2,4-triazol-3-yl]propanamide;
lxxi) 3,3-dicyclopropyl-N-[4-(3,5-dimethyl-1H-pyrazol-4-yl)phenyl]-2-(4-fluoro-5-phenyl-1H-imidazol-2-yl)propenamide;
or a pharmaceutically acceptable salt, hydrate, or solvate thereof.

19-21. (canceled)

22. A method of treating psoriasis, ankylosing spondylitis, spondyloarthritis, or psoriatic arthritis in a subject in need thereof, comprising administering to the subject a therapeutically effective amount of a compound according to claim 1.

23. A pharmaceutical composition comprising a compound according to claim 1 and one or more pharmaceutically acceptable vehicles, excipients, or pharmaceutically acceptable carriers.

24. (canceled)

25. A method of treating psoriasis, ankylosing spondylitis, spondyloarthritis, or psoriatic arthritis in a subject in need thereof comprising administering to the subject a therapeutically effective amount of a pharmaceutical composition according to claim 23.
